(19) United States
(12) Patent Application Publication
(10) Pub. No.: US 2019/0185836 A1
(43) Pub. Date: Jun. 20, 2019

(54) ENGINEERED SUBTILIGASE VARIANTS FOR VERSATILE, SITE-SPECIFIC LABELING OF PROTEINS

(71) Applicant: THE REGENTS OF THE UNIVERSITY OF CALIFORNIA, San Francisco, CA (US)

(72) Inventors: James A. WELLS, San Francisco, CA (US); Amy M. WEEKS, San Francisco, CA (US)

(21) Appl. No.: 16/329,176

(22) PCT Filed: Aug. 31, 2017
(86) PCT No.: PCT/US2017/049670

§ 371 Date: Feb. 27, 2019

Related U.S. Application Data
(60) Provisional application No. 62/383,265, filed on Sep. 2, 2016.
(60) Provisional application No. 62/398,898, filed on Sep. 23, 2016.
(60) Provisional application No. 62/547,496, filed on Aug. 18, 2017.

(51) Int. Cl. C12N 9/50; C12Q 1/37

(52) U.S. Cl. CPC C12N 9/50; C12Y 304/21062; C12Q 1/37

(57) ABSTRACT

In various embodiments, the invention provides subtiligase variants that recognize an N-terminus of a protein or peptide substrate that is different than the N-terminus that is recognized by the corresponding wild-type subtiligase. Also provided are methods and kits for using such subtiligase variants to site-specifically ligate a synthetic molecule comprising a peptide ester to a protein or peptide substrate of interest.

### CROSS-REFERENCES TO RELATED APPLICATIONS

The present application claims the benefit of U.S. Provisional Application Nos. 62/383,265, filed on Sep. 2, 2016, 62/398,898, filed on Sep. 23, 2016, and 62/547,496, filed Aug. 18, 2017, the disclosures of which are expressly incorporated by reference in their entirety for all purposes.

### STATEMENT AS TO RIGHTS TO INVENTIONS MADE UNDER FEDERALLY SPONSORED RESEARCH OR DEVELOPMENT

This invention was made with government support under grant nos. R01 CA191018 and R01 GM081051, awarded by the National Institutes of Health. The government has certain rights in the invention.

### FIELD OF THE INVENTION

The present invention relates to engineered subtiligase variant polypeptides having an altered N-terminal substrate specificity or an increased aminolysis-to-hydrolysis ratio (A/H ratio) compared to a wild-type (parental) subtiligase or wild-type (parental) stabiligase.

### BACKGROUND OF THE INVENTION

Sortase A is another peptide ligase that allows for N-terminal protein modification. However, this enzyme requires an N-terminal sequence of Gly-Gly-Gly and the technology is therefore unsuitable for modifying native or non-recombinant proteins. Additionally, sortase A-based bioconjugation generally requires super-stoichiometric amounts of enzyme for efficient bioconjugation, and the substrates to be conjugated on the non-prime site also require the presence of an LPXTG sequence, precluding its use for generation of native protein junctions. (WO2013/003555 A1).

A number of other enzymatic tagging technologies have also been reported and commercialized, including biotin ligase (BirA) (U.S. Pat. No. 7,172,877), 4′-phosphopantethienyl transferase (Sfp) (commercialized by New England Biolabs), formylglycine-generating enzyme (FGE) (U.S. Pat. No. 8,729,232), and lipoic acid ligase (Lp1A) (U.S. Pat. No. 8,137,925). While these enzymes work by different mechanisms and introduce a single protein modification, they all require the introduction of a multi-residue amino acid tag that is targeted by the conjugation reaction.

Chemical reactions that do not require enzyme catalysis are also used extensively for single-site protein bioconjugation. Among these are native chemical ligation, SNAP-tag (WO 2004/031404; commercialized by New England Biolabs) and Halo-tag (U.S. Pat. No. 8,742,086; commercialized by Promega). Native chemical ligation requires an N-terminal cysteine residue which has been demonstrated to interfere with proper protein function in many cases. SNAP-tag and Halo-tag require the fusion of an intact 19.4 kDa (SNAP-tag) or 33 kDa (Halo-tag) protein domain to achieve site-specific protein bioconjugation.

Other chemical reactions are also used for protein bioconjugation, including the modification of lysine residues with N-hydroxysuccinimide (NHS) esters or other amine-reactive reagents, and the modification of cysteine residues with maleimides or other thiol-reactive reagents. Lysine modification is disadvantageous compared to the invention because lysine is among the most common amino acids, so most proteins have multiple modification sites with amine-reactive reagents. Cysteine modification is disadvantageous compared to the invention because cysteine is the rarest amino acid, and many proteins contain no cysteines. Alternatively, proteins may contain >1 cysteine residue and genetic engineering must be used to remove cysteines to achieve single modification.

Enzyme-catalyzed peptide ligation is a powerful tool for site-specific protein bioconjugation, but stringent enzyme-substrate specificity often limits its utility. Native peptide ligase enzymes retain strict sequence requirements programmed by their biological functions, creating the need to genetically engineer the target ligation site.

The engineered peptide ligase subtiligase has broader sequence specificity and higher catalytic efficiency than natural peptide ligases, and represents an attractive tool for site-specific bioconjugation. However, qualitative specificity studies show that wild-type subtiligase harbors undesired sequence specificity that limits its utility for N-terminal bioconjugation, block-wise synthesis of proteins, and N-terminomics studies. Furthermore, poorly characterized N-terminal specificity makes the suitability of subtiligase for any particular application difficult to predict. For example, a recombinant antibody may be refractory to modification by wild-type subtiligase.

Site-specific chemical modification of proteins is an enabling technology for fields such as drug design, chemical biology, cell biology, and materials science. There is a present need for new technologies affording efficient, site-directed access to new and existing bioconjugates.

### BRIEF SUMMARY OF THE INVENTION

The present invention provides subtiligase variants with an altered N-terminal protein substrate specificity or an improved aminolysis-to-hydrolysis (A/H) ratio compared to a wild-type subtiligase or a wild-type stabiligase. The subtiligase variants can have an altered N-terminal protein substrate specificity or an improved A/H ratio compared to a naturally occurring subtilisin.

In some embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, M222, and a combination thereof, numbered in accordance with wild-type subtiligase.

In various embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, Y217, M222, and a combination thereof, numbered in accordance with wild-type subtiligase.

In certain embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/K/R/W, M222A, and a combination thereof, numbered in accordance with wild-type subtiligase

In various embodiments, the one or more amino acid substitutions in the subtiligase variant is selected form the group consisting of F189A/K/Q/R/S and Y217A/D/E/K/R/W. In some cases, the amino acid substitutions are F189A/K/Q/R/S and Y217A/D/E/K/R/W. In some embodiments, the subtiligase variant has the amino acid substitution M222A.

In some embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/R/W, M222A, and a combination thereof, numbered in accordance with wild-type subtiligase.

In some instances, the one or more amino acid substitutions are selected from the group consisting of F189A/K/Q/R/S and Y217A/D/E/R/W. In some cases, the amino acid substitutions are F189A/K/Q/R/S and Y217A/D/E/R/W. In some embodiments, the subtiligase variant has the amino acid substitution M222A.

In some embodiments, the subtiligase variant catalyzes ligation of the N-terminus of said protein substrate and a synthetic molecule comprising a peptide ester.

In some embodiments, the subtiligase variant catalyzes ligation of the N-terminus of said protein substrate and a synthetic molecule comprising a peptide thioester.

In certain embodiments, the altered N-terminal protein substrate specificity comprises an increased specificity for an acidic amino acid residue at the P1′ and/or the P2′ position of said protein substrate.

In some embodiments, the altered N-terminal protein substrate specificity comprises an increased specificity for a His, Lys, Ser or Arg residue at the P1′ position of said protein substrate.

In various embodiments, the altered N-terminal protein substrate specificity comprises an increased specificity for an aromatic, hydrophobic, polar, or acidic amino acid residue at the P1′ position and an acidic, basic, polar, or proline amino acid residue at the P2′ position of said protein substrate.

Provided herein is a nucleic acid encoding any one of the subtiligase variants described herein. Provided herein is an expression vector comprising any of the nucleic acids described herein. Also provided herein is a host cell comprising any one of the expression vectors described herein.

The present invention can be incorporated into a kit comprising any one of the subtiligase variants, any one of the nucleic acids, any one of the expression vectors, or any one of the host cells described herein, and a synthetic molecule for conjugating to the N-terminus of a protein substrate. In some embodiments, the synthetic molecule comprises a peptide ester or a peptide thioester. In some embodiments, the peptide ester or peptide thioester comprises a detectable moiety, therapeutic moiety, chemical moiety, drug moiety, binding moiety, nucleic acid, or reactive group.

In another aspect, the present invention provides a method of conjugating a synthetic molecule to the N-terminus of a protein substrate comprising contacting a protein substrate having a free α-amino group with one or more subtiligase variants having an altered N-terminal protein specificity or an improved A/H ratio compared to a wild-type subtiligase or a wild-type stabiligase, and a synthetic molecule under conditions to form a peptide bond between the synthetic molecule and the N-terminus of the protein substrate. In some embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, M222, and a combination thereof, numbered in accordance with wild-type subtiligase. In some embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, Y217, M222, and a combination thereof, numbered in accordance with wild-type subtiligase. In some embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/K/R/W, M222A, and a combination thereof, numbered in accordance with wild-type subtiligase. In some embodiments, the one or more amino acid substitutions are selected from the group consisting of F189A/K/Q/R/S and Y217A/D/E/K/R/W. In some cases, the amino acid substitutions are F189A/K/Q/R/S and Y217A/D/E/K/R/W. In some instances, the one or more amino acid substitutions are selected from the group consisting of F189A/K/Q/R/S and Y217A/D/E/K/R/W. In some embodiments, the subtiligase variant has the amino acid substitution M222A.

In some embodiments, the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/R/W, M222A, and a combination thereof, numbered in accordance with wild-type subtiligase.

In some instances, the one or more amino acid substitutions are selected from the group consisting of F189A/K/Q/R/S and Y217A/D/E/R/W. In some cases, the amino acid substitutions are F189A/K/Q/R/S and Y217A/D/E/R/W. In some embodiments, the subtiligase variant has the amino acid substitution M222A.

In various embodiments, the synthetic molecule comprises a peptide ester or a peptide thioester. In some instances, the peptide ester or the peptide thioester comprises a detectable moiety, therapeutic moiety, chemical moiety, drug moiety, binding moiety, nucleic acid, or reactive group.

In certain embodiments, the protein ester or the protein thioester further comprises an amino acid sequence comprising at least one unnatural amino acid residue.

In some embodiments, the protein substrate is present in a complex mixture. In some embodiments, the complex mixture is a biological sample. In some instances, the biological sample is a cell lysate, tissue extract, whole intact cells, whole blood, plasma, serum or other biological fluid.

The present invention addresses the need for site-specific bioconjugation reactions that proceed rapidly (such as on minute time scale); work optimally under mild conditions that preserve protein function; and produce chemical bonds that are stable over long periods of time in aqueous solution, cell lysate, and serum. The present invention also provides site-specific bioconjugation reaction methods that target a single site on a protein or peptide and do not require protein engineering to introduce genetic tags. Still also, the methods can be used on native or non-recombinant proteins. Bioconjugation reactions catalyzed by exemplary mutants of the invention generally are complete in less than one hour, proceed in aqueous solution at neutral pH, produce a stable, native peptide bond to the N-terminus with absolute selectivity over lysine side chains, and in combination, target many different N-terminal sequences, precluding the need for a genetic tag. In some embodiments, the mutants expand subtiligase substrate specificity to include sequences that have an acidic residue at the N-terminus, which is often the case for antibodies including antibody drugs.

In various embodiments, the invention provides a panel of subtiligase mutants (see, e.g., Figures and Examples herein) that alter the prime-side specificity of the original form of subtiligase. Exemplary mutants enable site-specific N-terminal bioconjugation of a variety of different moieties, e.g., probes, fluorophores, affinity handles, DNA barcodes, and drug payloads, to protein and peptide substrates that, in various embodiments, were previously inaccessible to subtiligase labeling due to incompatible N-terminal sequences. In some embodiments, these mutants can be used as a cocktail to achieve broadened specificity. In various embodiments, the invention also provides an algorithm or web-based tool, and means to execute the algorithm and achieve chemically relevant physical results using the algorithm, that facilitates the selection of a subtiligase mutant(s) for the N-terminal sequence(s) of interest. An exemplary bioconjugation reaction involves mixing the protein of interest with subtiligase and a specifically designed peptide ester that may include natural or unnatural amino acids and chemical modifications such as fluorophores, affinity handles, DNA barcodes, drug payloads, or reactive groups for further conjugation.

Other objects, advantages and embodiments of the invention will be apparent from the detailed description following.

### BRIEF DESCRIPTION OF THE DRAWINGS

FIG. 1A-FIG. 1B. Proteomic identification of ligation sites (PILS) applied to comprehensive characterization of subtiligase prime-side specificity. (FIG. 1A) The ligation reaction catalyzed by subtiligase accepts a peptide ester substrate, forms a thioester intermediate, and then transfers the peptide to an cc-amine containing acceptor peptide. (FIG. 1B) A schematic representation of the PILS strategy for comprehensive characterization of prime-side subtiligase specificity. Proteome-derived peptide libraries are generated by protease digestion of E. coli protein extract. The peptide libraries are used as substrates for modification by subtiligase and biotinylated peptide ester 1 (biotin-EEENLYFQ-Abu-glycolate-R). Biotinylated peptides are enriched on immobilized neutravidin and selectively eluted by cleavage with TEV protease, leaving an Abu mass tag on the N terminus of subtiligase substrates for positive identification. (FIG. 1C) Heatmap showing positional enrichment or de-enrichment of each amino acid at P1′-P5′ compared to the input peptide libraries. Data shows P1′ and P2′ positions are most discriminatin.

FIG. 2A-FIG. 2D. Defining and re-engineering subtiligase specificity for P1′ and P2′ residues. (FIG. 2A) PILS specificity map for the 400 possible N-terminal dipeptide sequences. Sequences that were not observed in the input library (18/400; 4.5%) are colored in grey. (FIG. 2B) Structure of the subtilisin-SSI complex showing the P4-P1 residues of SSI in purple and the P1′-P2′ residues of SSI in teal. Sites targeted for alanine-scanning mutagenesis with 7 Å of the catalytic triad are shown as grey spheres. Sites targeted for subsequent saturation mutagenesis are shown as light blue spheres. (FIG. 2C) Results of alanine-scanning mutagenesis. Each dot represents one dipeptide sequence. Dots are colored grey if there was no change in the mean enrichment score for the cluster; red if the sequences in the cluster were worse substrates for the mutant than the wild-type enzyme with a change in mean enrichment score of >2; and blue if the sequences in the cluster were better substrates for the mutant compared to the wild-type enzyme with a change in mean enrichment score of >2. Black bars show the mean and standard deviation of enrichment scores for each cluster. (FIG. 2D) Results of saturation mutagenesis studies. Left, position 217 (S1′ pocket); right, position 189 (S2′ pocket).

FIG. 3A-FIG. 3E. Scope of subtiligase-catalyzed N-terminal modification of folded proteins. (FIG. 3A) E. coli lysate assay to examine the scope of native proteins that can be labeled by subtiligase and variants. Lysates were prepared under native conditions and labeling with stabiligase, stabiligase-M222A, stabiligase-Y217K/M222A, or stabiligase-F189R/M222A and biotinylated ester 1. (FIG. 3B) Native proteins labeled at translational N termini (initiator Met or initiator Met removed, amino acid 2) and annotated signal peptide cleavage sites. (FIG. 3C) Weighted Venn diagram showing the overlap in N-termini labeled by each enzyme and 50% expansion of native proteins ligated by the new variants. (d) ESI mass spectra showing modification of GFP, a recombinant antibody, and commercial protein A by subtiligase or variants. (FIG. 3D) Quantitative labeling with azide-bearing peptides for native GFP, an antibody to GFP, and Protein A each with a different N-terminal sequence matched to their respective optimal subtiligase variants. (FIG. 3E) Ligation of the azide-bearing peptide onto GFP containing different N-terminal sequences tested with optimal subtiligase mutants. The heatmap shows the bioconjugation yield given by each mutant for each subtiligase variant tested. Sequence context of the mutant is shown at the bottom of the heatmap. ‘2×’ indicates that the labeling procedure was carried out a second time following desalting of the reaction mixture. The numerical value for the highest yield achieved for a particular N-terminal sequence is indicated.

FIG. 4A-FIG. 4F. Versatile reagents for one-step and modular protein modification strategies. (FIG. 4A) Strategy for one-step, subtiligase-catalyzed protein modification. Peptide ester 2 reacts with commercially available NHS esters, providing a convenient route to site-specific labeing reagents. (FIG. 4B) Strategy for modular subtiligase-catalyzed protein modification. Azide-bearing peptide ester 3 reacts with commercially available dibenzocyclooctynes (DBCOs), providing a convenient route for modular protein labeling. (FIG. 4C) ESI mass spectra for an anti-GFP rAb (αGFP) modified with a variety of different payloads using DBCO chemistry. MMAE, monomethyl auristatin E. (FIG. 4D) Subtiligase-modified rAbs maintain high-affinity antigen binding. (FIG. 4E) Dissociation constants measured using interferometry (Octet Red) for the anti-GFP rAb either unlabeled or labeled with different N-terminal peptides. Shown is the average and SD for X-measurements. (FIG. 4F) Cy3-α-GFP rAb staining of a HEK293T cell line modified for doxycycline-inducible expression of cell surface GFP.

FIG. 5A-FIG. 5D. Algorithmically selected cocktails of subtiligase mutants for cellular N terminomics. (FIG. 5A) Sequence space covered by wild-type subtiligase (left), all 72 subtiligase variants characterized in this study (center), and an algorithmically selected four-mutant cocktail. (FIG. 5B) Frequency of each amino acid at the P1′ position of apoptotic protease substrates (P1=D) as captured by stabiligase or the cocktail of stabiligase mutants. (FIG. 5C) Frequency of amino acids at the P1′ position of native proteins treated with methionine aminopeptidase, and labeled by stabiligase or the cocktail of stabiligase mutants followed by biotin capture and LC/MS (Abu tag at position 2). (FIG. 5D) Comparison of P1′ amino acids of signal peptidase substrates captured by stabiligase or the stabiligase cocktail compared to the predicted frequency of P1′ amino acids in predicted signal peptide cleavage sites. An enrichment score was calculated using the predicted cleavage sites as the reference set to quantify over- and under-representation of P1′ amino acids.

FIG. 6A-FIG. 6B. IceLogos for proteome-derived peptide libraries. (FIG. 6A) IceLogo for tryptic peptide acceptor library derived from the E. coli proteome. Amino acids that are enriched relative to natural abundance are shown above the line and amino acids that are de-enriched are shown below the line. (FIG. 6B) IceLogo for GluC peptide library derived from the E. coli proteome. IceLogos were generated using the IceLogo server (5) by using the first five amino acids of each peptide identified in the library as the experimental set and the pre-compiled Swiss-Prot composition for E. coli DH10B as the reference set. The scoring system was fold change with a p value of 0.05.

FIG. 7. Chemical structure of biotinylated subtiligase substrate. The subtiligase substrate, biotin-EEENLYFQ-glycolate-R-NH₂ (SEQ ID NO:18), includes a biotin tag for affinity purification (green), a TEV protease cleavage sequence (ENLYFQ, SEQ ID NO:19) for selective elution of subtiligated peptides (red), an aminobutyric acid (Abu) tag immediately following the TEV protease site, and a glycolate ester subtiligase acylation site (blue).

FIG. 8A-FIG. 8B. PILS specificity maps for subtiligase alanine scan mutants. Sequences that were enriched compared to the input library are colored in blue and sequences that were de-enriched compared to the input library are colored in red.

FIG. 9A-FIG. 9D. Kinetic analysis of subtiligase alanine mutants. (FIG. 9A) Design of FRET assay for subtiligase-catalyzed ligation and ester hydrolysis. The blue star represents the Pacific Blue fluorophore (ex=410 nm, em=455 nm), the green star represents a 5-/6-carboxyfluorescein (FAM) fluorophore (ex=495 nm, em=520 nm), and the quencher represents Dabcyl. (FIG. 9B) Fluorescence spectra of hydrolysis product (top left), ligation product and a variant lacking Pacific Blue (top right), quenched substrate in the presence or absence of subtiligase (bottom left), and quenched substrate and FAM-labeled nucleophile in the presence or absence of subtiligase. (FIG. 9C) Workflow for kinetics measurements. (FIG. 9D) Relative k_{cat}/K_{M} values for subtiligase mutants compared to wild-type subtiligase.

FIG. 10. Structural analysis of class I subtiligase mutants. Structure of the subtilisin-SSI complex showing the P4-P1 residues of SSI in purple and the P1′-P2′ residues of SSI in teal. The sites of class I alanine mutants are shown in light blue and the site of class II mutations are shown in light green. Positions of class I and class II mutations are labeled in black. Sites of other alanine mutations are shown in grey.

FIG. 11A-FIG. 11C. PILS specificity maps for Y217 and F189 mutants. Sequences that were enriched compared to the input library are colored in blue and sequences that were de-enriched compared to the input library are colored in red.

FIG. 12A-FIG. 12B. HPLC analysis of product ratio for subtiligase-M222A. Reaction mixtures containing 1 μM subtiligase variant, 350 μM suc-AAPFglcFG-NH₂ (ester substrate; SEQ ID NO:20) and 350 μM AF-NH₂ (nucleophile substrate) were incubated at room temperature for 1 h and analyzed by HPLC. (FIG. 12A) HPLC chromatograms for wild-type subtiligase and subtiligase-M222A. (FIG. 12B) Quantification of relative hydrolysis product and ligation product peak areas.

FIG. 13A and FIG. 13B. PILS specificity maps for additional subtiligase mutants. Sequences that were enriched compared to the input library are colored in blue and sequences that were de-enriched compared to the input library are colored in red.

FIG. 14. Subtiligase mutant specificity for native E. coli proteins. The set of N-terminal peptides identified using wild-type stabiligase for enrichment was used as the reference set. Sequences that were enriched compared to wild-type stabiligase are colored in blue and sequences that were de-enriched compared to wild-type stabiligase are colored in red.

FIG. 15. Subtiligase and mutant labeling of recombinant antibodies with extended N termini. An anti-GFP recombinant antibody (αGFP rAb) with the light chain N-terminus extended by zero, one (Gly), two (Gly-Gly), three (Gly-Gly-Gly), or four (Gly-Gly-Gly-Ser; SEQ ID NO: 21) residues was labeled with ester 2 (N₃AAPF-glycolyate-FG) and stabiligase-M222A or stabiligase-F189R/M222A. Modification yields of 11% (native N-terminus), 21% (Gly), 32% (Gly-Gly), 53% (Gly-Gly-Gly), and 62% (Gly-Gly-Gly-Ser) were observed, suggesting that N-terminal accessibility impacts modification yield.

FIG. 16. Subtiligase and mutant labeling of recombinant antibodies with orthogonal light chain and heavy chain N termini. An anti-GFP recombinant antibody (αGFP rAb) was labeled with ester 2 (N₃AAPF-glycolyate-FG) and subtiligase-Y217K or stabiligase. For the unmodified rAb sequence, no labeling was observed with stabiligase, while quantitative labeling was observed with subtiligase-Y217K (top panel). When an AFA extension was added to the N-terminus of the light chain, quantitative labeling of the light chain was observed with stabiligase, while quantitative labeling of both the light and heavy chains was observed with subtiligase-Y217K.

FIG. 17. Protein A labeling with a panel of subtiligase mutants. Protein A (50 μM) was labeled with 5 mM ester 2 (N₃AAPF-glycolyate-FG) and 1 μM subtiligase variant and analyzed by mass spectrometry to determine the extent of labeling. ‘2×’ indicates that the protein A reaction mixture was desalted and labeled a second time.

FIG. 18A-FIG. 18D. Characterization of synthetic peptides. Synthetic peptides were analyzed by LC-MS. Evaporative light scattering chromatograms are shown and peaks are labeled with the measured m/z values. (FIG. 18A) Biotin-EEENLYFQ-Abu-glc-R-NH₂. (FIG. 18B) N₃Ac-AAPC-glc-FG-NH₂ (SEQ ID NO: 22) (FIG. 18C) Suc-KAAPF-glc-FG-NH₂. (SEQ ID NO: 23) (FIG. 18D) NHS-biotin-modified suc-KAAPF-glc-FG-NH₂. Ac, acetyl; Suc, succinyl.

FIG. 19. P1′ and P2′ enrichment scores for wild-type subtiligase.

FIG. 20A -FIG. 20B. Masses of subtiligase mutants measured by LC-MS.

FIG. 21. GFP and GFP variant masses measured by LC-MS.

FIG. 22A-FIG. 22B. List of oligonucleotides used for plasmid construction and site-directed mutagenesis. FIG. 22A provides nucleic acid sequences for SEQ ID NOS: 24-59. FIG. 22B provides nucleic acid sequences for SEQ ID NOS: 60-83.

FIG. 23A-FIG. 23C. List of supplementary dataset information and ProteomeXchange Accession Numbers.

FIG. 24. Change in % ligation product formed compared to the hydrolysis product for various subtiligase mutants.

FIG. 25. Subtiligase substrate 1.

FIG. 26. Subtiligase substrate 2.

FIG. 27. Subtiligase substrate 3.

FIG. 28. Subtiligase substrate 4.

FIG. 29. Subtiligase substrate 5.

FIG. 30. Subtiligase substrate 6.

FIG. 31. Subtiligase substrate 7.

FIG. 32. Workflow and results of subtiligase engineering directed by Proteomic Identification of Ligation Sites (PILS).

### DETAILED DESCRIPTION OF THE INVENTION

### Introduction

Subtiligase as described in Jackson et al. (Science, 1994, 266(5):243-7; herein incorporated by reference in its entirety) and stabiligase as described in Chang et al. (Proc Natl Acad Sci USA, 1994, 91(26): 12544-12548; herein incorporated by reference in its entirety) harbor undesired sequence specificity that limits its ability to catalyze ligation to certain N-terminal sequence. The present invention is based, in part, on the discovery of novel engineered subtiligase variants that overcome this and other limitations.

In exemplary embodiments, the instant invention mitigates or eliminates the need for specific sequences to be introduced into a protein to facilitate bioconjugation while still targeting a single site. This is an improvement over the existing subtiligase technology because it targets many more of the 400 possible N-terminal dipeptide sequences.

The compositions, methods and kits outlined herein provide the advantages of eliminating the need for genetic engineering to introduce ligation tags, targeting a single site in a polypeptide, proceeding rapidly under mild conditions, and generating a stable peptide bond. In addition, the present invention can be used to introduce any number of different dipeptide sequences into the protein of interest depending on its N-terminal accessibility and other properties.

The present invention finds application both as a tool for researchers and in drug design, for example. In various embodiments, the subtiligase variants and specific substrates of the invention are included in a kit for site-specific protein modification. An exemplary application of such a kit include, but are not limited to, direct modification of antibodies, enzymes, and other proteins for use in imaging studies, antibody-drug conjugate screening, fluorescence assays, and affinity pulldowns. The invention also finds use in the production of antibody-drug conjugates as it, in various embodiments, targets a single-site, leading to uniform bioconjugation products that are predicted to be stable in serum.

### Definitions

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

As described in the present invention, the following terms will be employed, and are defined as indicated below.

The term “subtiligase” refers to proteins which have the enzymatic activity of being able to ligate esterified peptides site-specifically onto the N-termini (amino-termini) of proteins or peptides. Described herein are variants of a subtiligase polypeptide that have been engineered to exhibit advantageous features, such as enhanced stability and altered substrate specificity that are not exhibited by wild-type subtiligase or wild-type stabiligase.

The term “wild-type subtiligase” or “parental subtiligase” refers a non-naturally occurring mutant of any serine endoprotease subtilisin from Bacillus amyloliquefaciens, Bacillus lichenformis, Bacillus amylosaccaridicus, and other types of Bacillus, homologous serine protease from fungi, plant and higher animal species, or an engineered subtilisin BPN′ with the amino acid substitutions S221C/P225A (see, Abrahmsen et al., Biochemistry, 1991, 30:4151-4159). Non-limiting examples of wild-type subtiligases include those described in, for example, U.S. Pat. Nos. 4, 760, 025; 5,403,737; 5,629,173; 5,763,256; 5,736,512; 5,780,285; 5,741,664; and 5,837,516.

The term “wild-type stabiligase” or “parental stabiligase” refers a non-naturally occuring stabiligase enzyme as described in, for example, Chang et al., Proc. Natl. Acad. Sci. USA, 1994, 91:12544-12548 and Atwell et al, Proc. Natl. Acad. Sci. USA, 1999, 96:9497-9502. Exemplary embodiments of wild-type stabiligases include stabiligase polypeptides having the amino acid substitutions M50F/N76D/N109S/K213R/N218S or M50F/N76D/N109S/K213R/N218S/S125A/M124L.

The term “altered N-terminal protein substrate specificity,” in the context of a subtiligase variant refers to an increase in N-terminal protein substrate specificity such that the subtiligase variant described herein can N-terminally label a different set of more diverse set or larger set of protein substrates compared to a wild-type subtiligase or a wild-type stabiligase, or an ability to N-terminally label a protein substrate that can not be labeled by a wild-type subtiligase or a wild-type stabiligase.

The term “improved aminolysis to hydrolysis ratio,” “increased aminolysis to hydrolysis ratio,” “improved A/H ratio,” “increased A/H ratio,” in the context of a subtiligase variant refers an at least 1.5-fold, 2-fold, 5-fold, 10-fold, 50-fold, 100-fold, 200-fold, 500-fold, 1000-fold, or more increase in the ratio of aminolysis of the protein substrate to hydrolysis of the peptide ester, compared to a wild-type subtiligase or a wild-type stabiligase.

The term “peptide ester substrate” used in the context of subtiligase refers generally to any peptide ester or peptide thioester having a chemical moiety that is capable of being utilized during the enzymatic action of subtiligase that results in the specific labeling of the N-termini of proteins or peptides by subtiligase.

The term “peptide ester” refers generally to any peptide in which one carboxyl group of the peptide is esterified, i.e., is of the structure —CO—O—R. In some embodiments, a peptide ester can serve as a substrate for subtiligase such that the peptide is added to the α-amino group of polypeptides to form the structure —CO—NH—R, thus labeling the polypeptide. The esterified carboxyl terminus of the peptide ester, which serves as a subtiligase cleavage site (i.e., the site for the nucleophilic attack by a free sulfhydryl group on subtiligase as described herein). In some embodiments, a peptide ester can carry a detectable label and a site for proteolysis or another form of chemical cleavage (e.g., through introduction of photolabile, acid-labile, or base-labile functional groups). In some embodiments, the term “peptide ester” includes any peptide thioester such as any peptide in which one carboxyl group of the peptide is thioesterified, i.e., is of the structure —CO—S—R.

A “cleavable linker” when used in the context of a peptide ester of the present invention refers generally to any element contained within the peptide that can serve as a spacer and is labile to cleavage upon suitable manipulation. Accordingly, a cleavable linker may comprise any of a number of chemical entities, including amino acids, nucleic acids, or small molecules, among others. A cleavable linker may be cleaved by, for instance, chemical, enzymatic, or physical means. Non-limiting examples of cleavable linkers include protease cleavage sites and nucleic acid sequences cleaved by nucleases. Further, a nucleic acid sequence may form a cleavable linker between multiple entities in double stranded form by complementary sequence hybridization, with cleavage effected by, for instance, application of a suitable temperature increase to disrupt hybridization of complementary strands. Examples of chemical cleavage sites include the incorporation photolabile, acid-labile, or base-labile functional groups into peptides.

A “label,” “tag,” “detectable label,” “detectable moiety” or “detectable tag” includes a composition that can be detected by mass spectrometric, spectroscopic, photochemical, biochemical, immunochemical, or chemical means. For example, useful labels include radioactive isotopes (e.g., ³H, ³⁵S, ³²P ⁵¹Cr, or ¹²⁵1) stable isotopes (e.g., ¹³C, ¹⁵N, or ¹⁸O), fluorescent dyes, electron-dense reagents, enzymes (e.g., alkaline phosphatase, horseradish peroxidase, or others commonly used in an ELISA), biotin, digoxigenin, or haptens or epitopes and proteins for which antisera or monoclonal antibodies are available. In general, a tag or label as used in the context of the present invention is any entity that may be used to detect or isolate the product of the subtiligase ligation reaction. Thus, any entity that is capable of binding to another entity may be used in the practice of this invention, including without limitation, substrates for enzymes, epitopes for antibodies, ligands for receptors, and nucleic acids, which may interact with a second entity through means such as complementary base pair hybridization.

The term “complex mixture” refers generally to any composition that is composed of at least two or more proteins or peptides containing α-amines. A complex mixture can have at least two different proteins encoded by different genes; a complex mixture can be naturally occurring (e.g., a cell extract) or prepared (e.g., a formulation); a complex mixture can have recombinant, synthetic, or naturally occurring proteins or a mixture thereof. In many cases, a complex sample is one which displays a high degree of heterogeneity of proteins or peptides. Examples of complex mixtures include whole cells, cell extracts, partially purified cell extracts, tissues, bodily fluids, and animals, among others. Accordingly, in some embodiments, such complex mixtures comprise the naturally occurring proteins found in cells and tissues encoded by, for instance, different genes as found in the genomes of the source of the complex mixture (e.g., a cell or tissue extract or a bodily fluid such as serum). However, a complex mixture can also contain, as a component thereof, a recombinant protein or a purified protein or polypeptide either as an endogenous component (in the case of a recombinant protein), or as one added exogenously to the composition.

The term “conjugating” or “to conjugate” or “conjugation” refers to the process of linking, connecting, associating, or any combination thereof, two or more smaller entities, such as protein or protein fragments, to form a larger entity.

As used herein, the term “conjugate” refers to the association between atoms or molecules. The association can be direct or indirect. For example, a conjugate between a peptide and a detectable moiety can be direct, e.g., by covalent bond, or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In other embodiments, the peptide includes one or more reactive moieties, e.g., a covalent reactive moiety, as described herein (e.g., an amine reactive moiety). In other embodiments, the peptide includes a linker with one or more reactive moieties, e.g., a covalent reactive moiety, as described herein (e.g., an amine reactive moiety). Useful reactive moieties or functional groups used for conjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.;

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g., phosphines) to form, for example, phosphate diester bonds; and

(n) sulfones, for example, vinyl sulfone.

The reactive functional groups can be chosen such that they do not participate in, or interfere with, the chemical stability of the peptides or detectable moieties described herein.

A “labeled protein or peptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The term “isolated” as used herein with respect to nucleic acids, such as DNA or RNA, refers to molecules separated from other DNAs, or RNAs, respectively that are present in the natural source of the macromolecule. Isolated is meant to include nucleic acid fragments which are not naturally occurring as fragments and would not be found in the natural state. The term isolated as used herein also refers to a nucleic acid or peptide that is substantially free of cellular material, viral material, or culture medium when produced by recombinant DNA techniques, or chemical precursors, or other chemicals when chemically synthesized.

The term “recombinant nucleic acid molecule” refers to a non-naturally occurring nucleic acid molecule containing two or more linked polynucleotide sequences. A recombinant nucleic acid molecule can be produced by recombination methods, particularly genetic engineering techniques, or can be produced by a chemical synthesis method. A recombinant nucleic acid molecule can encode a fusion protein. The term “recombinant host cell” refers to a cell that contains a recombinant nucleic acid molecule. As such, a recombinant host cell can express a polypeptide from a “gene” that is not found within the native (non-recombinant) form of the cell.

Reference to a polynucleotide “encoding” a polypeptide means that, upon transcription of the polynucleotide and translation of the mRNA produced there from, a polypeptide is produced. The encoding polynucleotide is considered to include both the coding strand, whose nucleotide sequence is identical to an mRNA, as well as its complementary strand. It will be recognized that such an encoding polynucleotide is considered to include degenerate nucleotide sequences, which encode the same amino acid residues. Nucleotide sequences encoding a polypeptide can include polynucleotides containing introns as well as the encoding exons.

An expression control sequence refers to a nucleotide sequence that regulates the transcription or translation of a polynucleotide or the localization of a polypeptide to which it is operatively linked. Expression control sequences are “operatively linked” when the expression control sequence controls or regulates the transcription and, as appropriate, translation of the nucleotide sequence (e.g., a transcription or translation regulatory element, respectively), or localization of an encoded polypeptide to a specific compartment of a cell. Thus, an expression control sequence can be a promoter, enhancer, transcription terminator, a start codon (ATG), a splicing signal for intron excision and maintenance of the correct reading frame, a STOP codon, a ribosome binding site, or a sequence that targets a polypeptide to a particular location, for example, a cell compartmentalization signal, which can target a polypeptide to the cytosol, nucleus, plasma membrane, endoplasmic reticulum, mitochondrial membrane or matrix, chloroplast membrane or lumen, medial trans-Golgi cistemae, or a lysosome or endosome. Cell compartmentalization domains are well known in the art and include, for example, a peptide containing amino acid residues 1 to 81 of human type II membrane-anchored protein galactosyltransferase, or amino acid residues 1 to 12 of the presequence of subunit IV of cytochrome c oxidase (see also Hancock et al., EMBO J. 10:4033-4039, 1991; Buss et al., Mol. Cell. Biol. 8:3960-3963, 1988; and U.S. Pat. No. 5,776,689; each of which is incorporated herein by reference).

The term “operatively linked” or “operably linked” or “operatively joined” or the like, when used to describe chimeric (e.g., fusion) proteins, refer to polypeptide sequences that are placed in a physical and functional relationship to each other. In a most preferred embodiment, the functions of the polypeptide components of the chimeric protein are unchanged compared to the functional activities of the parts in isolation. As used herein, the fusion proteins of the invention can be in a monomeric state, or in a multimeric state (e.g., dimeric).

The term “nucleic acid” refers to deoxyribonucleotides or ribonucleotides and polymers thereof in either single- or double-stranded form, and complements thereof. The term refers to all forms of nucleic acids (e.g., gene, pre-mRNA, mRNA) and their polymorphic variants, alleles, mutants, and interspecies homologs. The term nucleic acid is used interchangeably with gene, cDNA, mRNA, oligonucleotide, and polynucleotide. The term encompasses nucleic acids that are naturally occurring or recombinant.

The term “identical” or “identity” or “percent identity,” or “sequence identity” in the context of two or more nucleic acids or polypeptide sequences that correspond to each other refer to two or more sequences or subsequences that are the same or have a specified percentage of amino acid residues or nucleotides that are the same (e.g., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical” and are embraced by the term “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists for a specified entire sequence or a specified portion thereof or over a region of the sequence that is at least about 25 amino acids or nucleotides in length, or more preferably over a region that is 50-100 amino acids or nucleotides in length. A corresponding region is any region within the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted (e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologus protein indicates that the protein comprises two or more subsequences that are not found in the same relationship to each other in nature (e.g., a fusion protein).

The terms “polypeptide,” “peptide,” and “protein” are used interchangeably herein to refer to polymers of amino acids of any length. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of functionality of the native full-length protein. The terms also encompass a modified amino acid polymer; for example, disulfide bond formation, glycosylation, lipidation, phosphorylation, methylation, carboxylation, deamidation, acetylation, or conjugation with a labeling component.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ.-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Hydrophilic Amino Acid” refers to an amino acid exhibiting a hydrophobicity of less than zero according to the normalized consensus hydrophobicity scale of Eisenberg et al., 1984, J. Mol. Biol. 179: 125-142. Genetically encoded hydrophilic amino acids include Thr (T), Ser (S), His (H), Glu (E), Asn (N), Gln (Q), Asp (D), Lys (K) and Arg (R).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

“Basic Amino Acid” refers to a hydrophilic amino acid having a side chain pK value of greater than 7. Basic amino acids typically have positively charged side chains at physiological pH due to association with hydrogen ion. Genetically encoded basic amino acids include His (H), Arg I and Lys (K).

“Polar Amino Acid” refers to a hydrophilic amino acid having a side chain uncharged at physiological pH, but which has at least one bond in which the pair of electrons shared in common by two atoms is held more closely by one of the atoms. Genetically encoded polar amino acids include Asn (N), Gln (Q), Ser (S) and Thr (T).

“Hydrophobic Amino Acid” refers to an amino acid exhibiting a hydrophobicity of greater than zero according to the normalized consensus hydrophobicity scale of Eisenberg, 1984, J. Mol. Biol. 179:125-142. Exemplary hydrophobic amino acids include Ile (I), Phe (F), Val (V), Leu (L), Trp (W), Met (M), Ala (A), Gly (G),Tyr (Y), Pro (P), and proline analogues.

“Aromatic Amino Acid” refers to a hydrophobic amino acid with a side chain having at least one aromatic or heteroaromatic ring. The aromatic or heteroaromatic ring may contain one or more substituents such as-OH,—SH, —CN, —F, —Cl, —Br, —I, —NO₂, —NO, —NH₂, —NHR, —NRR, —C (O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₁-C₂₁)) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe (F), Tyr (Y) and Trp (W).

“Nonpolar Amino Acid” refers to a hydrophobic amino acid having a side chain uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A).

“Aliphatic Amino Acid” refers to a hydrophobic amino acid having an aliphatic hydrocarbon side chain. Genetically encoded aliphatic amino acids include Ala (A), Val (V), Leu (L) and Ile (I).

The term “non-natural” or “unnatural” with regard to amino acids can include any amino acid molecule not included as one of the 20 natural amino acids as well as any modified or derivatized amino acid known to one of skill in the art. Non-naturally amino acids can include but are not limited to β-alanine, α-amino butyric acid, γ-amino butyric acid, γ-(aminophenyl) butyric acid, α-amino isobutyric acid, ε-amino caproic acid, 7-amino heptanoic acid, β-aspartic acid, aminobenzoic acid, aminophenyl acetic acid, aminophenyl butyric acid, γ-glutamic acid, cysteine (ACM), ε-lysine, methionine sulfone, norleucine, norvaline, ornithine, d-ornithine, p-nitro-phenylalanine, hydroxy proline, 1,2,3,4,-tetrahydroisoquinoline-3-carboxylic acid, and thioproline.

“Biological sample” as used herein is a sample of cells, biological tissue, or fluid that is to be tested for the occurrence of proteolysis or the presence, more generally, of polypeptides of interest in the sample. Among the cells that can be examined are cancer cells, cells stimulated to under apoptosis, and cells at different stages of development, among others. The biological tissues of this invention include any of the tissues that comprise the organs of an organism. The biological sample can be derived from any species including bacteria, yeasts, plants, invertebrates, and vertebrate organisms. The fluid of this invention can be any fluid associated with a cell or tissue. Such fluids may include the media in which cells are cultured as well as the fluid surrounding tissues and organs, as well as the fluid comprising the circulatory system of invertebrates and vertebrates (e.g., body fluids such as whole blood, serum, plasma, cerebrospinal fluid, urine, lymph fluids, and various external secretions of the respiratory, intestinal and genitourinary tracts, tears, saliva, milk, white blood cells, myelomas, and the like). An “extracellular fluid” refers generally to any fluid found exterior to cells. Such fluids may include all of the fluids described above. In certain embodiments, such fluids may further include cellular debri, for example from lysed cells, including membrane-bound and cytosolic proteins. A biological sample used in the present invention may be from a suitable organism, for example a mammal such as a mouse, rat, hamster, guinea pig, rabbit, sheep, goat, pig, monkey, human, and the like.

### DETAILED DESCRIPTION OF THE EMBODIMENTS

### A. Engineered Subtiligase Variants

Synthetic subtiligase variants described herein can ligate esterified or thioesterified peptides site-specifically onto the N-terminus of a protein or fragment thereof. In the first step of a subtiligase reaction using a peptide ester substrate, a free sulhydryl group on the subtiligase enzyme serves as a nucleophile to affect a nucleophilic attack on the carbonyl carbon atom of the ester moiety of the peptide ester substrate, resulting in the release of an alcohol leaving group. The peptide ester substrate can carry a label or a tag. In the second step of the reaction, the carbonyl carbon of the thioester linkage between the peptide substrate and the subtiligase enzyme is then subject to nucleophilic attack by the α-amino group of a protein substrate. This reaction results in a covalent adduct comprising the peptide (or the labeled peptide) linked to the α-amino group on a protein via an amide bond. Subtiligase can be used for selective labeling of the N-termini of proteins.

Subtiligases can recognize the first four amino acids after the ester bond of a peptide ester substrate or the thioester bond of a peptide thioester substrate (e.g., the P1, P2, P3 and P4 positions) and the first four amino acids after the free α-amine of the protein substrate (e.g., the P1′, P2′, P3′ and P4′ positions). Subtiligase variants described herein can bind the non-prime and prime sides of a peptide ester substrate and a protein substrate simultaneously. In some cases, such a subtiligase can sample the prime side of the substrate independently of the non-prime side following enzyme acylation. Thus, substrate specificity and/or sequence selectivity can be determined according to the amino acid residues on the non-prime side of the substrate. Alternatively, substrate specificity and/or sequence selectivity can be determined according to the amino acid residues on the prime side of the substrate.

Provided herein are subtiligase variants having an expanded or broadened N-terminal specificity compared to the N-terminal specificity of wild-type subtiligase or wild-type stabiligase. In addition, the subtiligase variants can have an increased or improved aminolysis to hydrolysis ratio (A/H ratio) compared to a wild-type subtiligase or a wild-type stabiligase.

Wild-type subtiligase can be a native subtilisin from Bacillus or a homolog thereof from any other species including fungi, plant, animal or human, or a subtilisin BPN variant with two amino acid substitutions: S211C and P225A (Jackson et. al., supra). This subtilisin variant (referred to as a wild-type or parental subtiligase) showed a 500-fold increase in synthesis over hydrolysis ratio (S/H ratio) as compared to wild-type subtilisin BPN. The average ligating yield was around 66% and hydrolysis of the oligopeptide acyl donor C-terminal ester remained substantial. It has poor stability against organic co-solvents that are required to solubilize the oligopeptide fragments. It also has poor stability against enhanced temperature and against denaturating agents, which are often needed for successful oligopeptide condensation.

Wild-type or parental stabiligase refers to a subtiligase mutant with five additional mutations (M50F, N76D, N109S, K213R and N218S). This mutant is more stable than wild-type subtiligase and appears moderately more resistant to sodium dodecasulfate and guanidinium hydrochloride. Similar to the wild-type subtiligase, hydrolysis is a major side reaction for this enzyme (see, e.g., Chang et al., supra).

Subtiligase variants described herein have a broadened or increased N-terminal substrate specificity or an increased A/H ratio compared to a wild-type subtiligase or a wild-type stabiligase. In some cases, the subtiligase variant prefers a specific amino acid residue at the P1′ position and/or the P2′ position of the protein substrate that is not preferred or is not tolerated by a wild-type subtiligase or a wild-type subtiligase stabiligase. In some cases, the A/H ratio of the subtiligase variant is at least 1.5-fold, 2-fold, 5-fold, 10-fold, 50-fold, 100-fold, 200-fold, 500-fold higher than the A/H ratio of a wild-type subtiligase or a wild-type subtiligase stabiligase.

In some embodiments, the subtiligase variant comprises an amino acid sequence that is at least 90%, e.g., 90%, 91%, 92%, 93%, 94%, 95%, 96%, 97%, 98% or 99% identical to the amino acid sequence of wild-type subtiligase or wild-type stabiligase or SEQ ID NO:1, SEQ ID NO:2 or SEQ ID NO:3 or SEQ ID NO:4, or a fragment thereof, and has one or more amino acid substitutions selected from the group consisting of D60, N62, S63, H67, S125, L126, F189, M222, and a combination thereof, numbered in accordance with wild-type subtiligase. In some cases, the variant has 1, 2, 3, 4, 5, 6, 7, 8, 9, 10, or more amino acid substitutions. In various embodiments, the subtiligase variant comprises an amino acid sequence that is at least 90%, e.g., 90%, 91%, 92%, 93%, 94%, 95%, 96%, 97%, 98% or 99% identical to SEQ ID NO:1, or a fragment thereof, and has one or more amino acid substitutions selected from the group consisting of D60, N62, S63, H67, S125, L126, F189, M222, and a combination thereof, numbered in accordance with wild-type subtiligase. In some embodiments, the subtiligase variant comprises an amino acid sequence that is at least 90%, e.g., 90%, 91%, 92%, 93%, 94%, 95%, 96%, 97%, 98% or 99% identical to SEQ ID NO:2, or a fragment thereof, and has one or more amino acid substitutions selected from the group consisting of D60, N62, S63, H67, S125, L126, F189, M222, and a combination thereof, numbered in accordance with wild-type subtiligase. In other embodiments, the subtiligase variant comprises an amino acid sequence that is at least 90%, e.g., 90%, 91%, 92%, 93%, 94%, 95%, 96%, 97%, 98% or 99% identical to SEQ ID NO:3 or a fragment thereof, and has one or more amino acid substitutions selected from the group consisting of D60, N62, S63, H67, S125, L126, F189, M222, and a combination thereof, numbered in accordance with wild-type subtiligase. In certain embodiments, the subtiligase variant comprises an amino acid sequence that is at least 90%, e.g., 90%, 91%, 92%, 93%, 94%, 95%, 96%, 97%, 98% or 99% identical to SEQ ID NO:4, or a fragment thereof, and has one or more amino acid substitutions selected from the group consisting of D60, N62, S63, H67, S125, L126, F189, M222, and a combination thereof, numbered in accordance with wild-type subtiligase.

In some embodiments, the subtiligase variant comprises an amino acid sequence that is at least 90%, e.g., 90%, 91%, 92%, 93%, 94%, 95%, 96%, 97%, 98% or 99% identical to the amino acid sequence of wild-type subtiligase or wild-type stabiligase or SEQ ID NO:1, SEQ ID NO:2 or SEQ ID NO:3 or SEQ ID NO:4, or a fragment thereof, and has one or more amino acid substitutions selected from the group consisting of D60, N62, S63, H67, S125, L126, F189, Y217, M222, and a combination thereof, numbered in accordance with wild-type subtiligase. In some cases, the variant has 1, 2, 3, 4, 5, 6, 7, 8, 9, 10 or more amino acid substitutions. In various embodiments, the subtiligase variant comprises an amino acid sequence that is at least 90%, e.g., 90%, 91%, 92%, 93%, 94%, 95%, 96%, 97%, 98% or 99% identical to SEQ ID NO:1 or a fragment thereof, and has one or more amino acid substitutions selected from the group consisting of D60, N62, S63, H67, S125, L126, F189, Y217, M222, and a combination thereof, numbered in accordance with wild-type subtiligase. In certain embodiments, the subtiligase variant comprises an amino acid sequence that is at least 90%, e.g., 90%, 91%, 92%, 93%, 94%, 95%, 96%, 97%, 98% or 99% identical to SEQ ID NO:2 or a fragment thereof, and has one or more amino acid substitutions selected from the group consisting of D60, N62, S63, H67, S125, L126, F189, Y217, M222, and a combination thereof, numbered in accordance with wild-type subtiligase. In particular embodiments, the subtiligase variant comprises an amino acid sequence that is at least 90%, e.g., 90%, 91%, 92%, 93%, 94%, 95%, 96%, 97%, 98% or 99% identical to SEQ ID NO:3 or a fragment thereof, and has one or more amino acid substitutions selected from the group consisting of D60, N62, S63, H67, S125, L126, F189, Y217, M222, and a combination thereof, numbered in accordance with wild-type subtiligase. In some embodiments, the subtiligase variant comprises an amino acid sequence that is at least 90%, e.g., 90%, 91%, 92%, 93%, 94%, 95%, 96%, 97%, 98% or 99% identical to SEQ ID NO:4 or a fragment thereof, and has one or more amino acid substitutions selected from the group consisting of D60, N62, S63, H67, S125, L126, F189, Y217, M222, and a combination thereof, numbered in accordance with wild-type subtiligase. For instance, the variants can have the following amino acid substitutions: D60, N62, S63, H67, S125, L126, F189, Y217, M222, D60/N62, D60/563, D60/H67, D60/S125, D60/L126, D60/F189, D60/Y217, D60/M222, N62/563, N62/H67, N62/S125, N62/L126, N62/F189, N62/Y217, N62/M222, S63/H67, S63/S125, S63/L126, S63/F189, S63/Y217, S63/M222, H67/S125, H67/L126, H67/F189, H67/Y217, H67/M222, S125/L126, S125/F189, S125/Y217, S125/M222, F189/Y217, F189/M222, Y217/M222, D60/N62/563, H67/N62/563, S125/N62/S63, L126/N62/S63, F189/N62/S63, Y217/N62/563, M222/N62/S63, D60/S63/H67, N62/S63/H67, S125/S63/H67, L126/S63/H67, F189/S63/H67, Y217/S63/H67, M222/S63/H67, D60/N62/S63/H67, S 125/L126/F189/Y217, L126/F189/Y217/M222, D60/N62/S63/H67/S125, S125/L126/F189/Y217/M222, D60/N62/S63/H67/S125/L126/F189, N62/S63/H67/S125/L126/F189/Y217/M222, or D60/N62/S63/H67/S 125/L126/F189/Y217/M222.

In various embodiments, the subtiligase variant comprises an amino acid sequence that is at least 90%, e.g., 90%, 91%, 92%, 93%, 94%, 95%, 96%, 97%, 98% or 99% identical to the amino acid sequence of wild-type subtiligase or wild-type stabiligase or SEQ ID NO:1, SEQ ID NO:2 or SEQ ID NO:3 or SEQ ID NO:4, or a fragment thereof, and has one or more amino acid substitutions selected from the group consisting of D60A, N62A, S63A, H67A, S125A, L126A, F189A/K/Q/R/S, M222A, and a combination thereof, numbered in accordance with wild-type subtiligase. In some cases, the variant has 1, 2, 3, 4, 5, 6, 7, 8, 9, 10, or more amino acid substitutions. In various embodiments, the subtiligase variant comprises an amino acid sequence that is at least 90%, e.g., 90%, 91%, 92%, 93%, 94%, 95%, 96%, 97%, 98% or 99% identical to SEQ ID NO:1 or a fragment thereof, and has one or more amino acid substitutions selected from the group consisting of D60A, N62A, S63A, H67A, S125A, L126A, F189A/K/Q/R/S, M222A, and a combination thereof, numbered in accordance with wild-type subtiligase. In certain embodiments, the subtiligase variant comprises an amino acid sequence that is at least 90%, e.g., 90%, 91%, 92%, 93%, 94%, 95%, 96%, 97%, 98% or 99% identical to SEQ ID NO:2 or a fragment thereof, and has one or more amino acid substitutions selected from the group consisting of D60A, N62A, S63A, H67A, S125A, L126A, F189A/K/Q/R/S, M222A, and a combination thereof, numbered in accordance with wild-type subtiligase. In particular embodiments, the subtiligase variant comprises an amino acid sequence that is at least 90%, e.g., 90%, 91%, 92%, 93%, 94%, 95%, 96%, 97%, 98% or 99% identical to SEQ ID NO:3 or a fragment thereof, and has one or more amino acid substitutions selected from the group consisting of D60A, N62A, S63A, H67A, S125A, L126A, F189A/K/Q/R/S, M222A, and a combination thereof, numbered in accordance with wild-type subtiligase. In some embodiments, the subtiligase variant comprises an amino acid sequence that is at least 90%, e.g., 90%, 91%, 92%, 93%, 94%, 95%, 96%, 97%, 98% or 99% identical to SEQ ID NO:4 or a fragment thereof, and has one or more amino acid substitutions selected from the group consisting of D60A, N62A, S63A, H67A, S125A, L126A, F189A/K/Q/R/S, M222A, and a combination thereof, numbered in accordance with wild-type subtiligase. For instance, the variant can have the following amino acid substitutions: D60A, N62A, S63A, H67A, S125A, L126A, F189A, F189K, F189Q, F189R, F189S, M222A, D60A/N62A, D60A/S63A, D60A/H67A, D60A/S125A, D60A/L126A, D60A/F189A, D60A/F189K, D60A/F189Q, D60A/F189R, D60A/F189S, D60A/M222A, N62A/S63A, N62A/H67A, N62A/S125A, N62A/L126A, N62A/F189A, N62A/F189K, N62A/F189Q, N62A/F189R, N62A/F189S, N62A/M222A, S63A/H67A, S63A/L126A, S63A/F189A, S63A/F189K, S63A/F189Q, S63A/F189R, S63A/F189S, S63A/M222A, L126A/F189A, L126A/F189K, L126A/F189Q, L126A/F189R, L126A/F 189S, L126A/M222A, F189A/M222A, F189K/M222A, F189Q/M222A, F189R/M222A, or F189S/M222A, and in some embodiments, one or more additional amino acid substitutions selected from D60A, N62A, S63A, H67A, S125A, L126A, F189A, F189K, F189Q, F189R, F189S, or M222A.

In some other embodiments, the subtiligase variant comprises an amino acid sequence that is at least 90%, e.g., 90%, 91%, 92%, 93%, 94%, 95%, 96%, 97%, 98% or 99% identical to the amino acid sequence of wild-type subtiligase or wild-type stabiligase or SEQ ID NO:1, SEQ ID NO:2 or SEQ ID NO:3 or SEQ ID NO:4, or a fragment thereof, and has one or more amino acid substitutions selected from the group consisting of D60A, N62A, S63A, H67A, S125A, L126A, F189A/K/Q/R/S, Y217A/D/E/K/R/W, M222A, and a combination thereof, numbered in accordance with wild-type subtiligase. In some cases, the variant has 1, 2, 3, 4, 5, 6, 7, 8 or 9 amino acid substitutions. In some embodiments, the subtiligase variant comprises an amino acid sequence that is at least 90%, e.g., 90%, 91%, 92%, 93%, 94%, 95%, 96%, 97%, 98% or 99% identical to SEQ ID NO:1, SEQ ID NO:2 or SEQ ID NO:3 or SEQ ID NO:4 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In certain embodiments, the subtiligase variant comprises an amino acid sequence having at least 90%, e.g., 90%, 91%, 92%, 93%, 94%, 95%, 96%, 97%, 98% or 99% sequence identity to SEQ ID NO:1 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In various embodiments, the subtiligase variant comprises an amino acid sequence having at least 90%, e.g., 90%, 91%, 92%, 93%, 94%, 95%, 96%, 97%, 98% or 99% sequence identity to SEQ ID NO:2 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In particular embodiments, the subtiligase variant comprises an amino acid sequence having at least 90%, e.g., 90%, 91%, 92%, 93%, 94%, 95%, 96%, 97%, 98% or 99% sequence identity to SEQ ID NO:3 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In some embodiments, the subtiligase variant comprises an amino acid sequence having at least 90%, e.g., 90%, 91%, 92%, 93%, 94%, 95%, 96%, 97%, 98% or 99% sequence identity to SEQ ID NO:3 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. For instance, the variant can have an amino acid substitution including, but not limited to, D60A, N62A, S63A, H67A, S125A, L126A, F189A/K/Q/R/S, Y217A/D/E/K/R/W, M222A, D60A/N62A, D60A/S63A, D60A/H67A, D60A/S125A, D60A/L126A, D60A/(F189A/K/Q/R/S), D60A/(Y217A/D/E/K/R/W), D60A/M222A, N62A/S63A, N62A/H67A, N62A/S125A, N62A/L126A, N62A/(F189A/K/Q/R/S), N62A/(Y217A/D/E/K/R/W), N62A/M222A, S63A/H67A, S63A/S125A, S63A/L126A, S63A/(F189A/K/Q/R/S), S63A/(Y217A/D/E/K/R/W), S63A/M222A, H67A/S125A, H67A/L126A, H67A/(F189A/K/Q/R/S), H67A/(Y217A/D/E/K/R/W), H67A/M222A, S125A/L126A, S125A/(F189A/K/Q/R/S), S125A/(Y217A/D/E/K/R/W), S125A/M222A, (F189A/K/Q/R/S)/(Y217A/D/E/K/R/W), (F189A/K/Q/R/S)/M222A, (Y217A/D/E/K/R/W)/M222A, D60A/N62A/S63A, H67A/N62A/S63A, S125A/N62A/S63A, L126A/N62A/S63A, (F189A/K/Q/R/S)/N62A/S 63A, (Y217A/D/E/K/R/W)/N62A/S63A, M222A/N62A/S63A, D60A/S63A/H67A, N62A/S63A/H67A, S125A/S63A/H67A, L126A/S63A/H67A, (F189A/K/Q/R/S)/S63A/H67A, (Y217A/D/E/K/R/W)/S63A/H67A, M222A/S63A/H67A, D60A/N62A/S63A/H67A, S125A/L126A/(F189A/K/Q/R/S)/(Y217A/D/E/K/R/W), L126A/(F189A/K/Q/R/S)/(Y217A/D/E/K/R/W)/M222A, D60A/N62A/S63A/H67A/S 125A, S125A/L126A/(F189A/K/Q/R/S)/(Y217A/D/E/K/R/W)/M222A, D60A/N62A/S63A/H67A/S125A/L126A/(F189A/K/Q/R/S), N62A/S63A/H67A/S125A/L126A/(F189A/K/Q/R/S)/(Y217A/D/E/K/R/W)/M222A, D60A/N62A/S63A/H67A/S125A/L126A/(F189A/K/Q/R/S)/(Y217A/D/E/K/R/W)/M222A, and the like. In some embodiments, the variant outlined herein has a F189A, F189K, F189Q, F189R, F189S, Y217A, Y217D, Y217E, Y217K, Y217R, or Y217W substitution. Optionally, the variant has a F189A/K/Q/R/S substitution and aY217A/D/E/K/R/W substitution. In other words, the variant has a F189A or F189K or F189Q or F189R or F189S substitution, and a Y217A or Y217D or Y217E or Y217K or Y217R or Y217W substitution. In some embodiments, the subtiligase variant has a F189A/Y217A substitution, F189A/Y217D substitution, F189A/Y217E substitution, F189A/Y217K substitution, F189A/Y217R substitution, F189A/Y217W substitution, F189K/Y217A substitution, F189K/Y217D substitution, F189K/Y217E substitution, F189K/Y217K substitution, F189K/Y217R substitution, F189K/Y217W substitution, F189Q/Y217A substitution, F189Q/Y217D substitution, F189Q/Y217E substitution, F189Q/Y217K substitution, F189Q/Y217R substitution, F189Q/Y217W substitution, F189R/Y217A substitution, F189R/Y217D substitution, F189R/Y217E substitution, F189R/Y217K substitution, F189R/Y217R substitution, F189R/Y217W substitution, F189S/Y217A substitution, F189S/Y217D substitution, F189S/Y217E substitution, F189S/Y217K substitution, F189S/Y217R substitution, F189S/Y217W substitution. Any of the variants described herein can have an M222A substitution. In various embodiments, the subtiligase variant has a F189A/M222A, F189K/M222A, F189Q/M222A, F189R/M222A, F189S/M222A, Y217A/M222A,Y217D/M222A, Y217E/M222A, Y217R/M222A, Y217W/M222A,F189A/Y217A/M222A substitution, F189A/Y217D/M222A substitution, F189A/Y217E/M222A substitution, F189A/Y217K/M222A substitution, F189A/Y217R/M222A substitution, F189A/Y217W/M222A substitution, F189K/Y217A/M222A substitution, F189K/Y217D/M222A substitution, F189K/Y217E/M222A substitution, F189K/Y217K/M222A substitution, F189K/Y217R/M222A substitution, F189K/Y217W/M222A substitution, F189Q/Y217A/M222A substitution, F189Q/Y217D/M222A substitution, F189Q/Y217E/M222A substitution, F189Q/Y217K/M222A substitution, F189Q/Y217R/M222A substitution, F189Q/Y217W/M222A substitution, F189R/Y217A/M222A substitution, F189R/Y217D/M222A substitution, F189R/Y217E/M222A substitution, F189R/Y217K/M222A substitution, F189R/Y217R/M222A substitution, F189R/Y217W/M222A substitution, F189S/Y217A/M222A substitution, F189S/Y217D/M222A substitution, F189S/Y217E/M222A substitution, F189S/Y217K/M222A substitution, F189S/Y217R/M222A substitution, or F189S/Y217W/M222A substitution.

In some instances, the subtiligase variant comprises an amino acid sequence having at least 90%, at least 91%, at least 92%, at least 93%, at least 94%, at least 95%, at least 96%, at least 97%, at least 98% or at least 99% sequence identity to the amino acid sequence of wild-type subtiligase or wild-type stabiligase or SEQ ID NO:1, SEQ ID NO:2 or SEQ ID NO:3 or SEQ ID NO:4, or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In some cases, the variant has 1, 2, 3, 4, 5, 6, 7, 8, 9, 10 or more amino acid substitutions.

In some embodiments, the subtiligase variant comprises an amino acid sequence having at least 90%, at least 91%, at least 92%, at least 93%, at least 94%, at least 95%, at least 96%, at least 97%, at least 98% or at least 99% sequence identity to the amino acid sequence of wild-type subtiligase or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In certain embodiments, the subtiligase variant comprises an amino acid sequence having at least 90%, at least 91%, at least 92%, at least 93%, at least 94%, at least 95%, at least 96%, at least 97%, at least 98% or at least 99% sequence identity to the amino acid sequence of wild-type subtiligase or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In particular embodiments, the subtiligase variant comprises an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 1 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In one embodiment, the subtiligase variant comprises an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 2 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In another embodiment, the subtiligase variant comprises an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 3 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase. In another embodiment, the subtiligase variant comprises an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 4 or a fragment thereof, and has one or more amino acid substitutions selected from the group consisting of D60A, N62A, S63A, H67A, S125A, L126A, F189A, F189K, F189Q, F189R, F189S, Y217A, Y217D, Y217E, Y217K, Y217R, Y217W, M222A, and a combination thereof, numbered in accordance with wild-type subtiligase.

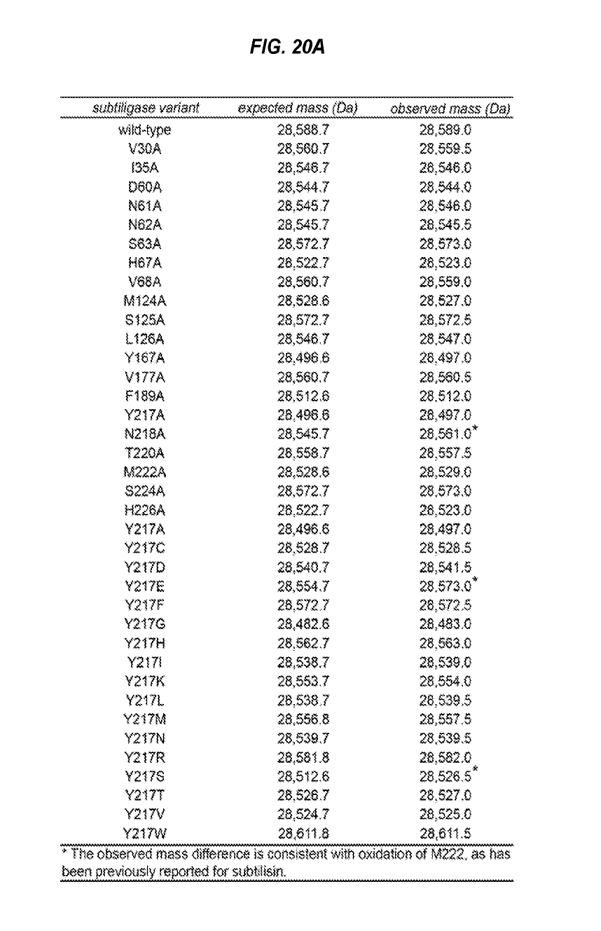

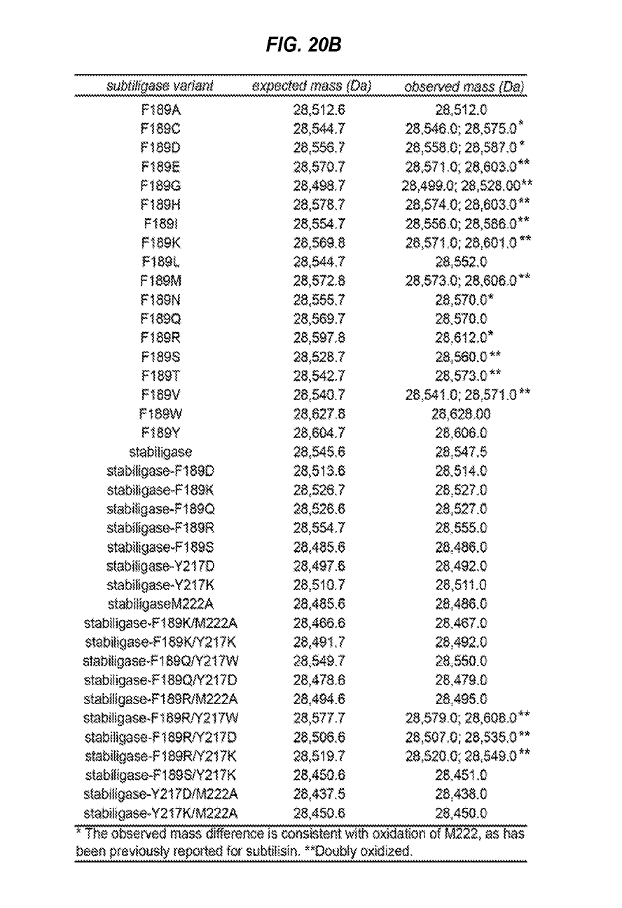

Useful subtiligase variants include, but are not limited to those described in FIG. 20. The subtiligase variants outlined herein can be based on wild-type subtiligase, wild-type stabiligase, SEQ ID NO: 1, SEQ ID NO: 2, SEQ ID NO: 3, or SEQ ID NO: 4, and have any of the following amino acid substitutions: V30A, I35A, D60A, N61A, N62A, S63A, H67A, V68A, M124A, S125A, L126A, Y167A, V177A, F189A, F189C, F189D, F189E, F189G, F189H, F1891, F189K, F189L, F189M, F189N, F189Q, F189R, F189S, F189T, F189V, F189W, F189Y, Y217A, N218A, T220A, M222A, S224A, H226A, Y217A, Y217C, Y217D, Y217E, Y217F, Y217G, Y217H, Y2171, Y217K, Y217L, Y217M, Y217N, Y217R, Y217S, Y217T, Y217V, or Y217W. Further still, the subtiligase variants outlined herein can be based on wild-type subtiligase, wild-type stabiligase, SEQ ID NO: 1, SEQ ID NO: 2, SEQ ID NO: 3, or SEQ ID NO: 4 and have any of the following amino acid substitutions: F189D, F189K, F189Q, F189R, F189S, Y217D, Y217K, M222A, F189D/M222A, F189K/M222A, F189Q/M222A, F189R/M222A, F189S/M222A, F189A/Y217A, F189A/Y217D, F189A/Y217E, F189A/Y217K, F189A/Y217R, F189A/Y217W, F189K/Y217A, F189K/Y217D, F189K/Y217E, F189K/Y217K, F189K/Y217R, F189K/Y217W, F189Q/Y217A, F189Q/Y217D, F189Q/Y217E, F189Q/Y217K, F189Q/Y217R, F189Q/Y217W, F189R/Y217A, F189R/Y217D, F189R/Y217E, F189R/Y217K, F189R/Y217R, F189R/Y217W, F189S/Y217A, F189S/Y217D, F189S/Y217E, F189S/Y217K, F189S/Y217R, F189S/Y217W, Y217A/M222A, Y217D/M222A, Y217E/M222A, Y217K/M222A, Y217R/M222A, or Y217W/M222A.

Accordingly, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 1 and a F189K/Y217K substitution. In some cases, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 2 and a F189K/Y217K substitution. In other cases, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 3 and a F189K/Y217K substitution. In various cases, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 4 and a F189K/Y217K substitution.

In some embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 1 and a F189R/Y217K substitution. In particular embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 2 and a F189R/Y217K substitution. In other embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 3 and a F189R/Y217K substitution. In various embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 4 and a F189R/Y217K substitution.

In some embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 1 and a F189R/Y217D substitution. In some embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 2 and a F189R/Y217D substitution. In other embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 3 and a F189R/Y217D substitution. In various embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 4 and a F189R/Y217D substitution.

In various embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 1 and a F189S/Y217K substitution. In some embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 2 and a F189S/Y217K substitution. In other embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 3 and F189S/Y217K substitution. In various embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 4 and a F189S/Y217K substitution.

In certain embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 1 and a F189Q/Y217D substitution. In some embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 2 and a F189Q/Y217D substitution. In other embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 3 and F189Q/Y217D substitution. In various embodiments, the subtiligase variant can comprise or consist of an amino acid sequence having at least 90%, at least 91%, at least 92%, at least 93%, at least 94%, at least 95%, at least 96%, at least 97%, at least 98% or at least 99% sequence identity to the amino acid sequence of SEQ ID NO: 4 and a F189Q/Y217D substitution.

In some instances, the subtiligase variant provided herein can include M50F, N76D, N109S, M124L, S125A, K213R, N218S, and M222A substitutions (numbering based on wild-type subtiligase). In other instances, the subtiligase variant provided herein can include M50F, N76D, N109S, M124L, S125A, K213R, Y217K, N218S, and M222A substitutions. In some instances, the subtiligase variant provided herein can include M50F, N76D, N109S, M124L, S125A, F189R, K213R, N218S, and M222A substitutions (numbering based on wild-type subtiligase).

Substrate specificity of a subtiligase or variant thereof is determined, in part, by the binding of the peptide ester and the amine nucleophile of the protein substrate. The last four amino acid residues of the peptide ester substrate (e.g., P1, P2, P3 and P4) the first three amino acid residues of the free N-terminus of the protein substrate (e.g., P1′, P2′ and P3′) directly interact with subtiligase, and affect its specificity. The enzyme and variants thereof bind the peptide ester and the protein substrate in a conformation from about four amino acid residues N-terminal of the cleaved bond of the peptide ester (P1 to P4) to about three residues C-terminal to the cleaved bond (P1′ to P3′).

Some amino acid residues at the P1′ position are preferred by a subtiligase variant such that, for example, ligation efficiency increases, ligation rate decreases, and the like. Other amino acid residues at this position can be less preferred.

Subtiligase variants can have N-terminal specificity for protein substrates with an acidic amino acid residue (e.g., Asp or Glu), a basic amino acid residue (e.g., His, Lys, or Arg), or a Ser residue at the P1′ position. Such variants can have a greater preference for an acidic amino acid residue (e.g., Asp or Glu), a basic amino acid residue (e.g., His, Lys, or Arg), or a Ser residue at the P1′ position compared to wild-type subtiligase or wild-type stabiligase.

In some instances, subtiligase variants can have N-terminal specificity for protein substrates with an acidic amino acid residue (e.g., Asp or Glu), a basic amino acid residue (e.g., His, Lys, or Arg), or a Ser residue at the P2′ position. Some variants can have an increased preference for an acidic amino acid residue (e.g., Asp or Glu), a basic amino acid residue (e.g., His, Lys, or Arg), or a Ser residue at the P2′ position compared to wild-type subtiligase or wild-type stabiligase. Other variants can have a reduced preference for an acidic amino acid residue (e.g., Asp or Glu), a basic amino acid residue (e.g., His, Lys, or Arg), or a Ser residue at the P2′ position compared to wild-type subtiligase or wild-type stabiligase.

In other instances, subtiligase variants can have N-terminal specificity for protein substrates with a small amino acid residue (e.g., Gly or Ala), Met or Arg at the P1′ position and an aromatic amino acid residue (e.g., Phe, Tyr, or Trp), His, Met, Cys, or Ala at the P2′ position. In yet other instances, subtiligase variants can have N-terminal specificity for protein or peptide substrates with any amino acid except Asp or Glu at the P1′ position and an aromatic amino acid residue (e.g., Phe, Tyr, or Trp) at the P2′ position. In some cases, subtiligase variants can have N-terminal specificity for protein or peptide substrates with an aromatic amino acid residue (e.g., Phe, Tyr, or Trp), a large hydrophobic amino acid residue (e.g., Phe, Leu, Ile or Val), a polar amino acid residue (e.g., Ser, Thr, Cys, Tyr, Asn or Gln), or an acidic amino acid residue (e.g., Asp or Glu) at the P1′ position, and an acidic amino acid residue (e.g., Asp or Glu), a basic amino acid (e.g., His, Lys or Arg) residue, or a polar amino acid residue (e.g., Ser, Thr, Cys, Tyr, Asn or Gln) at the P2′ position. In some cases, subtiligase variants can have N-terminal specificity for protein or peptide substrates with an acidic amino acid residue (e.g., Asp or Glu) at the P1′ position and/or at the P2′ position. The N-terminal specificity of the subtiligase variant can be increased compared to that of a wild-type subtiligase or wild-type stabiligase.

Subtiligase variants outlined herein can have an enhanced or increased A/H ratio compared to a wild-type subtiligase or wild-type stabiligase such that the ratio is at least 1.5-fold, 2-fold, 5-fold, 10-fold, 50-fold, 100-fold, 200-fold, 500-fold, 1000-fold, or more higher. As such, in the conjugation reaction the variants have a greater preference for aminolysis compared to hydrolysis. Methods for determining an A/H ratio of an enzyme are described in, for example, Abrahmsen et al., Biochemistry, 1991, 30:4151-4159. The method can include determining the amount of aminolysis product to the amount of hydrolysis product.

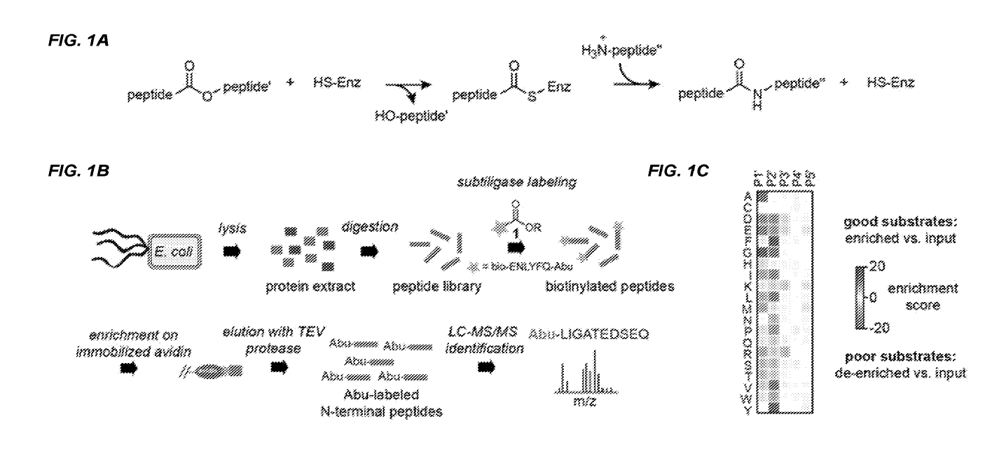

N-terminal specificity can be measured using any method known to those in the art. Useful methods include, but are not limited to, the proteomic identification of cleavage site (PICS) method or the proteomic identification of ligation sites (PILS) method. The PICS method has been used to determine the prime-side specificity of subtilisin, and the PILS method has been used to determine the prime-side specificity of subtiligase, stabiligase, and variants thereof. Both methods are described in the Example and FIGS. 1A and 1B.

Useful methods for N-terminal labeling of proteins using subtiligases include those described in, for example, Yoshihara et al., Bioorg Med Chem Lett, 2008, 18(22): 6000-6003, Wildes and Wells, Proc Natl Acad Sci USA, 2010, 107(10):4561-4566, and Wiita et al., Methods Enzymol, 2014, 544:327-358, the contents of which are hereby incorporated by reference in their entirely for all purposes, and in the Example below.

Provided herein are various engineered peptide ligases with defined sequence specificities (such as prime-side specificies) and are based on subtiligase or stabiligase. Such engineered peptide ligases are useful for site-specific modification of protein N-termini.

### B. Peptide Esters or Peptide Thioesters

A useful peptide ester or peptide thioester can be any synthetic peptide in which one carboxyl group of the peptide is esterified, i.e., is of the structure —CO—O—R, or thiesterified, i.e., is of the structure —CO—S—R, respectively. The peptide ester or peptide thioester can serve as a substrate for a subtiligase described herein such that the peptide is added to the α-amino group of a polypeptide or peptide substrate to form the structure —CO—NH—R, thus labeling the polypeptide or peptide substrate.

The ester/thioester-containing synthetic peptide can carry any tag for labeling the N-terminus of the protein or peptide substrate. Non-limiting examples of a tag include an amino acid, a peptide, a protein, a polynucleotide, a carbohydrate, a metal atom, a contrast agent, a catalyst, a cytotoxic molecule, a non-polypeptide polymer, a recognition element, a small molecule, a lipid, a linker, a detectable label, an epitope, an antigen, a therapeutic agent, a drug payload, a toxin, a radioisotope, a particle, a viral particle, a click chemistry handle, a binding molecule, a targeting molecule, and an antibody or derivative thereof.

The tag can be a detectable moiety (detectable label), therapeutic moiety, chemical moiety, drug moiety, binding moiety, and nucleic acid. A detectable moiety can be radioisotopes, stable isotopes, fluorophores, heavy metals, among others.

As used herein, a “detectable label” includes a moiety that has at least one element, isotope, or functional group incorporated into the moiety which enables detection of the molecule, e.g., a protein or polypeptide, or other entity, to which the label is attached. Labels can be directly attached (i.e., via a bond) or can be attached by a tether (such as, for example, an optionally substituted alkylene; an optionally substituted alkenylene; an optionally substituted alkynylene; an optionally substituted heteroalkylene; an optionally substituted heteroalkenylene; an optionally substituted heteroalkynylene; an optionally substituted arylene; an optionally substituted heteroarylene; or an optionally substituted acylene, or any combination thereof, which can make up a tether). It will be appreciated that the label may be attached to or incorporated into a molecule, for example, a protein, polypeptide, or other entity, at any position.

In general, a label can fall into any one (or more) of five classes: a) a label which contains isotopic moieties, which may be radioactive or heavy isotopes, including, but not limited to, ²H, ³H, ¹³C, ¹⁴C, ¹⁵N, ¹⁸F, ³¹F, ³²F, ³⁵S, ⁶⁷Ga, ⁷⁶Br, ^{99m}Tc (Tc-99m), ^{m}In, ¹²³I, ¹²⁵I, ¹³¹I, ¹⁵³Gd, ¹⁶⁹Yb, and ¹⁸⁶Re; b) a label which contains an immune moiety, which may be antibodies or antigens, which may be bound to enzymes (e.g., such as horseradish peroxidase); c) a label which is a colored, luminescent, phosphorescent, or fluorescent moieties (e.g., such as the fluorescent label fluoresceinisothiocyanat (FITC); d) a label which has one or more photo affinity moieties; and e) a label which is a ligand for one or more known binding partners (e.g., biotin-streptavidin, FK506-FKBP). In certain embodiments, a label comprises a radioactive isotope, preferably an isotope which emits detectable particles, such as β particles. In certain embodiments, the label comprises a fluorescent moiety. In certain embodiments, the label is the fluorescent label fluoresceinisothiocyanat (FITC). In certain embodiments, the label comprises a ligand moiety with one or more known binding partners. In certain embodiments, the label comprises biotin. In some embodiments, a label is a fluorescent polypeptide (e.g., GFP or a derivative thereof such as enhanced GFP (EGFP)) or a luciferase (e.g., a firefly, Renilla, or Gaussia luciferase). It will be appreciated that, in certain embodiments, a label may react with a suitable substrate (e.g., a luciferin) to generate a detectable signal. Non-limiting examples of fluorescent proteins include GFP and derivatives thereof, proteins comprising chromophores that emit light of different colors such as red, yellow, and cyan fluorescent proteins, etc. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mK02, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols (Methods of biochemical analysis, v. 47). Wiley-Interscience, Hoboken, N.J., 2006, and/or Chudakov, D M, et al, Physiol Rev. 90(3): 1103-63, 2010 for discussion of GFP and numerous other fluorescent or luminescent proteins. In some embodiments, a label comprises a dark quencher, e.g., a substance that absorbs excitation energy from a fluorophore and dissipates the energy as heat.

A chemical moiety can be a reactive group such as a reactive group that can be used for downstream chemical reactions. Non-limiting examples of a reactive group include a thiol for reactivity with electrophiles, an azide, alkyne, or cyclooctyne for click chemistry, an amine for reactivity with amine-reactive reagents such as NHS esters, and an alkoxyamine for reactivity with aldehydes.

A drug moiety can be a small molecule, toxin, therapeutic agent, protein or fragment thereof, antibody or fragment thereof, or antibody derivative or variant thereof.

A binding moiety can be biotin, albumin, a peptide, antigen, antibody, a viral particle, a recognition element, or a targeting moiety that binds to a tumor antigen or a protein expressed on the surface of a cell.

One skilled in the art will recognize that the chemical structure of the peptide ester tag and peptide thioester tag can be selected to facilitate downstream purification, identification, and/or quantification of the labeled protein in specific applications.

Any peptide ester with the following generic elements may be used in the practice of the present invention: tag-linker-peptide sequence-esterified carboxyl terminus. Similarly, any useful peptide thioester can have the following generic elements: tag-linker-peptide sequence-thioesterified carboxyl terminus. The skilled artisan will recognize that the location of the label within this structure may be varied without affecting the operation of the present invention. The generic structure of these elements may optionally contain a protease cleavage site or other cleavable moiety to facilitate the ready removal of the label added to the α-amino group of a protein or polypeptide. Such removal also greatly facilitates downstream mass spectrometric analysis of labeled proteins or peptides.

The peptide ester can have a site for proteolysis or any other form of chemical cleavage. Non-limiting examples of a cleavage moiety or cleavable linker include ENLYFQSY (SEQ ID NO:5), ENLYFQSK (SEQ ID NO:6), ENLYFQSA (SEQ ID NO:7), AAPY (SEQ ID NO:8), AAPK (SEQ ID NO:9), and AAPA (SEQ ID NO:10). Optional protease cleavage sites that may be used in the practice of this invention include, but are not limited to: the site for TEV protease: EXXYXQ(S/G/A) (SEQ ID NO:11), where X corresponds to any amino acid; the site for rhinovirus 3C protease: E(T/V)LFQGP (SEQ ID NO:12); the site for enterokinase: DDDDK (SEQ ID NO:13); the site for Factor Xa: I(D/E)GR(SEQ ID NO:14); the site for thrombin: LVPR (SEQ ID NO:15); the site for furin: RXXR(SEQ ID NO:16), where X corresponds to any amino acid; and the site for granzyme B: IEPD (SEQ ID NO:17). Some examples of the many possible moieties that may be used to esterify the carboxyl terminus of the peptide are: HO—CH₂—CO—X, where X is any amino acid, in the case of glycolate esters; HO—CHCH₃—CO—X, where X is any amino acid, in the case of lactate esters; HO—R, where R is an alkyl or aryl substituent; and HS—R, where R is an alkyl or aryl substituent. In some embodiments, a peptide ester carries a label and a site for proteolysis or another form of chemical cleavage (e.g., through introduction of photolabile, acid-labile, or base-labile functional groups).

The amino acid sequence of the peptide ester or peptide thioester can contain natural amino acid residues, noncanonical amino acid residues, unnatural amino acid residues, and the like. An unnatural amino acid residue can be found at any position of the peptide sequence.

A peptide ester or peptide thioester can be synthesized using any method known to those in the art, including. but not limited to, solid phase fMOC chemistry modified for an ester bond (Braisted et al., Methods in Enzymology, 1997, 289:298-313; Jackson et al., Science, 1994, 266:243-247).

### C. Protein or Peptide Substrates

Subtiligase variants described herein can be used ligate or label the N-terminus (i.e., α-amino group) of proteins or peptides in complex mixtures. A complex mixture can include mixture comprising at least two different polypeptides or peptides. In some cases, the complex mixture is a biological sample such as, but not limited to, whole intact cells, cell extracts, tissue extracts, cell lysates, cell culture media from cell cultures, whole blood, serum, plasma, and any other bodily fluid.

The protein or peptide substrate can be from a biological sample including, but not limited to, a sample of cells, biological tissue, or biological fluid. The biological sample can be derived from any species including bacteria, yeast, plants, invertebrates, and vertebrates. The biological tissue can be obtained from any part or organ of the body, e.g., human body. Non-limiting examples of a biological fluid include whole blood, serum, plasma, cerebrospinal fluid, urine, lymph fluids, tears, saliva, mucus, sweat, bile breast milk, amniotic fluid, other fluids of the respiratory, intestinal and genitourinary tracts, and the like.

A protein or peptide substrate can be from an extract of isolated cells or isolated tissue. For instance, the isolated cells or tissue can be cancer cells such as tumor cells, a circulating tumor cells, bone marrow cells, a tissue biopsy, etc.

Methods for making such an extract are known in the art and are described, for example, in Scopes, R. K., PROTEIN PURIFICATION: PRINCIPLES AND PRACTICE, Springer-Verlag: NewYork (1982). In general, cells are disrupted to release and solubilize intracellular contents, followed by centrifugation to remove insoluble material, such as cell membranes and organelles. For tissue culture cells, a lysis buffer which may contain a detergent (e.g., Triton X-100, NP-40, among others) may be used. For adherent tissue culture cells, cell disruption can be accomplished by the process of scraping cells in the presence of the lysis buffer from culture plates using, for example, a rubber policeman. Other mechanical means can also be used to effect cell disruption. For example, cells can be lysed using a Dounce homogenizer. As recognized by the skilled artisan, additional mechanical means may be needed to prepare cell extracts from tissues, such as homogenization in a blender or sonication. The proteins in a complex mixture such as biological sample can be solubilized into an appropriate buffer for the subtiligase reaction.

The protein or peptide substrates can be antibodies, enzymes, therapeutic proteins, drug proteins, proteins for imaging, binding proteins, variants thereof, derivatives thereof, and fragments thereof, or any protein of interest.

In some embodiments, the protein substrate for the subtiligase variant described herein has a small amino acid, Met, or Arg at the P1′ position and an aromatic amino acid, a large hydrophobic amino acid, His, Met, Cys, or Ala in the P2′ position. In certain embodiments, the protein substrate for the subtiligase variant described herein has any amino acid except Asp or Glu at the P1′ position and an aromatic amino acid in the P2′ position. In various embodiments, the protein substrate for the subtiligase variant described herein has an aromatic amino acid, a large hydrophobic amino acid, a polar amino acid, or an acidic amino acid at the P1′ position and an acidic amino acid, a basic amino acid, or a polar amino acid in the P2′ position. In other instances, the protein substrate for the subtiligase variant described herein does not have an aromatic amino acid, a large hydrophobic amino acid, a polar amino acid, or an acidic amino acid at the P1′ position and an acidic amino acid, a basic amino acid, or a polar amino acid in the P2′ position. In some embodiments, the protein substrate for the subtiligase variant described herein has an acidic amino acid at the P1′ position. In other embodiments, the protein substrate for the subtiligase variant described herein has an acidic amino acid in the P2′ position.

In some embodiments, the subtiligase variant comprises an amino acid sequence having at least 90% (e.g., 90%, 91%, 92%, 93%, 94%, 95%, 96%, 97%. 98%, or 99%) sequence identity to a wild-type subtiligase, a wild-type stabiligase, SEQ ID NO: 1, SEQ ID NO: 2, SEQ ID NO: 3, or SEQ ID NO: 4, has a D60A substitution, and has a specificity for a protein substrate with an aromatic amino acid, a large hydrophobic amino acid, a polar amino acid, or an acidic amino acid at the P1′ position and an acidic amino acid, a basic amino acid, or a polar amino acid in the P2′ position, a protein substrate with an acidic amino acid at the P1′ position, or a protein substrate with an acidic amino acid in the P2′ position. In some cases, such variants have reduced specificity for a protein substrate with a small amino acid, Met, or Arg at the P1′ position and an aromatic amino acid, a large hydrophobic amino acid, His, Met, Cys, or Ala in the P2′ position, or a protein substrate with any amino acid except Asp or Glu at the P1′ position and an aromatic amino acid in the P2′ position. In various embodiments, the subtiligase variant comprises an amino acid sequence having at least 90% (e.g., 90%, 91%, 92%, 93%, 94%, 95%, 96%, 97%. 98%, or 99%) sequence identity to a wild-type subtiligase, a wild-type stabiligase, SEQ ID NO: 1, SEQ ID NO: 2, SEQ ID NO: 3, or SEQ ID NO: 4 and a D60A or N62A or S63A or N125A substitution, and has a k_{cat}/K_{M} of at least 2-fold higher, e.g., at least 2-fold higher, at least 4-fold higher, at least 5-fold higher, at least 6-fold higher, at least 7-fold higher, at least 8-fold higher, at least 9-fold higher, at least 10-fold higher, or higher compared to the wild-type enzyme. In some cases, the k_{cat}/K_{M} of at least 2-fold-10-fold higher, e.g., at least 2-fold-10-fold higher, at least 3-fold-10-fold higher, at least 4-fold-10-fold higher, at least 5-fold-10-fold higher, at least 6-fold-10-fold higher, at least 7-fold-10-fold higher, at least 8-fold-10-fold higher, at least 9-fold-10-fold higher, at least 2-fold-3-fold higher, at least 2-fold-4-fold higher, at least 2-fold-5-fold higher, at least 2-fold-6-fold higher, at least 2-fold-8-fold higher, at least 2-fold-8-fold higher, at least 2-fold-5-fold higher, at least 3-fold-7-fold higher, or at least 4-fold-10-fold higher compared to the wild-type enzyme.

In some embodiments, the subtiligase variant comprises an amino acid sequence having at least 90% (e.g., 90%, 91%, 92%, 93%, 94%, 95%, 96%, 97%. 98%, or 99%) sequence identity to a wild-type subtiligase, a wild-type stabiligase, SEQ ID NO: 1, SEQ ID NO: 2, SEQ ID NO: 3, or SEQ ID NO: 4, and a H76A or L126A substitution, and has a specificity for a protein substrate with a small amino acid, Met, or Arg at the P1′ position and an aromatic amino acid, a large hydrophobic amino acid, His, Met, Cys, or Ala in the P2′ position or a protein substrate a protein substrate with any amino acid except Asp or Glu at the P1′ position and an aromatic amino acid in the P2′ position. In some cases, such variants have reduced specificity for a protein substrate with an aromatic amino acid, a large hydrophobic amino acid, a polar amino acid, or an acidic amino acid at the P1′ position and an acidic amino acid, a basic amino acid, or a polar amino acid in the P2′ position, or a protein substrate with an acidic amino acid at the P1′ position and an acidic amino acid in the P2′ position. In various embodiments, the subtiligase variant comprises an amino acid sequence having at least 90% sequence identity to a wild-type subtiligase, a wild-type stabiligase, SEQ ID NO: 1, SEQ ID NO: 2, SEQ ID NO: 3, or SEQ ID NO: 4, and a H76A or L126A substitution, and has a k_{cat}/K_{M} of at least 2-fold higher, e.g., at least 2-fold higher, at least 4-fold higher, at least 5-fold higher, at least 6-fold higher, at least 7-fold higher, at least 8-fold higher, at least 9-fold higher, at least 10-fold higher, at least 11-fold higher, at least 12-fold higher, at least 13-fold higher, at least 14-fold higher, at least 15-fold higher, at least 15-fold higher, at least 20-fold higher, at least 25-fold higher, at least 50-fold higher or greater compared to the wild-type enzyme. In some cases, the k_{cat}/K_{M} of at least 2-fold-20-fold higher or more, e.g., at least 2-fold-20-fold higher, at least 4-fold-20-fold higher, at least 4-fold-16-fold higher, at least 5-fold-20-fold higher, at least 10-fold-20-fold higher, at least 15-fold-20-fold higher, at least 2-fold-5-fold higher, at least 2-fold-10-fold higher, at least 2-fold-15-fold higher, at least 5-fold-10-fold higher, at least 5-fold-15-fold higher, or greater compared to the wild-type enzyme.

In certain embodiments, the subtiligase variant comprises an amino acid sequence having at least 90% (e.g., 90%, 91%, 92%, 93%, 94%, 95%, 96%, 97%. 98%, or 99%) sequence identity to a wild-type subtiligase, a wild-type stabiligase, SEQ ID NO: 1, SEQ ID NO: 2, SEQ ID NO: 3, or SEQ ID NO: 4, and a F189A substitution, and has a specificity for a protein substrate with an aromatic amino acid, a large hydrophobic amino acid, a polar amino acid, or an acidic amino acid at the P1′ position and an acidic amino acid, a basic amino acid, or a polar amino acid in the P2′ position or a protein substrate with an acidic amino acid in the P2′ position.

In certain embodiments, the subtiligase variant comprises an amino acid sequence having at least 90% (e.g., 90%, 91%, 92%, 93%, 94%, 95%, 96%, 97%. 98%, or 99%) sequence identity to a wild-type subtiligase, a wild-type stabiligase, SEQ ID NO: 1, SEQ ID NO: 2, SEQ ID NO: 3, or SEQ ID NO: 4, and a Y217A substitution, and has a specificity for a protein substrate with an acidic amino acid at the P1′ position.

### D. Site-Specific N-Terminal Labeling of Proteins and Peptides

The variants are useful for semi-synthesis of proteins, total synthesis of non-naturally occurring proteins, and N-terminal labeling of proteins for proteomic studies, for diagnosis or prognosis of disease, and for determining biological outcome. In some cases, a nucleic acid such as a DNA barcode can be conjugated to proteins of interest.

In some aspects, the present invention provides a method of conjugating a synthetic molecule to the N-terminus of a protein substrate. The method comprises contacting a protein substrate having a free α-amino group with one or more subtiligase variants outlined herein which have an altered N-terminal protein specificity or an enhanced A/H ratio compared to a wild-type subtiligase or a wild-type stabiligase, and a synthetic molecule under conditions to form a peptide bond between the synthetic molecule and the N-terminus of the protein substrate. In some embodiments, the protein substrate comprises a small amino acid, Met, or Arg at the P1′ position and an aromatic amino acid, a large hydrophobic amino acid, His, Met, Cys, or Ala in the P2′ position. In certain embodiments, the protein substrate comprises any amino acid except Asp or Glu at the P1′ position and an aromatic amino acid in the P2′ position. In various embodiments, the protein substrate comprises an aromatic amino acid, a large hydrophobic amino acid, a polar amino acid, or an acidic amino acid at the P1′ position and an acidic amino acid, a basic amino acid, or a polar amino acid in the P2′ position. In other embodiments, the protein substrate comprises an acidic amino acid at the P1′ position. In another embodiment, the protein substrate comprises an acidic amino acid in the P2′ position. The protein substrate can be found in a complex mixture comprising one or more proteins, such as a biological sample. Non-limiting examples of a biological sample include a cell lysate, tissue extract, whole cells, whole blood, plasma, serum, and other biological fluids.

The synthetic molecule can be a peptide ester or a peptide thioester. In some case, the peptide ester also includes amino acid sequence comprising at least one, e.g., 1, 2, 3, 4, 5, 6 or more unnatural amino acids.

Any reaction condition can be used with the compositions, methods and kits described herein to label free α-amino groups of proteins or peptides. Generally, any conditions under which ester or thioester reagents are stable to degradation and hydrolysis in complex samples; conditions under which subtiligase is stable and active; and conditions under which protein and polypeptide N-termini are free and available to react with the linkage formed after the reaction of subtiligase with ester or thioester reagents are favored for the practice of this invention. Useful conditions include those that promote aminolysis.

The subtiligase reaction can be performed under nondenaturing conditions, such as in conditions with no free detergent or denaturant. In addition, the reaction can be performed at or above neutral pH (i.e., pH 7.0). In certain embodiments, the reaction is performed at pH 7.0 or higher, e.g., pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6, pH 8.7, pH 8.8, pH 8.9, pH 9.0, pH 9.1, pH 9.2, pH 9.3, pH 9.4, pH 9.5, pH 9.6, pH 9.7, pH 9.8, pH 9.9, or higher. In some embodiments, the subtiligase reaction is performed at a pH range of about pH 7.0-9.0, e.g., about pH 7.0-9.0, pH 7.1-9.0, pH 7.2-9.0, pH 7.3-9.0, pH 7.4-9.0, pH 7.5-9.0, pH 7.6-9.0, pH 7.7-9.0, pH 7.8-9.0, pH 7.9-9.0, pH 8.0-9.0, pH 8.1-9.0, pH 8.2-9.0, pH 8.3-9.0, pH 8.4-9.0, pH 8.5-9.0, pH 8.6-9.0, pH 8.7-9.0, pH 8.8-9.0, pH 8.9-9.0, pH 8.0-8.9, pH 8.1-8.9, pH 8.2-8.9, pH 8.3-8.9, pH 8.4-8.9, pH 8.5-8.9, pH 8.6-8.9, pH 8.7-8.9, pH 8.8-8.9, pH 7.0-8.9, pH 7.1-8.9, pH 7.2-8.9, pH 7.3-7.9, pH 7.4-8.9, pH 7.5-8.9, 7 pH 7.6-8.9, pH 7.7-8.9, pH 7.8-8.9, or pH 7.9-8.9.

N-terminal labeled proteins can be evaluated using methods known to those in the art such as mass spectrometry (e.g., LC-MS/MS), Western blotting, and ELISA assay. Methods for determining the efficiency and/or specificity for the labeling procedure or the A/H ratio of a subtiligase variant are described in, for example, Jackson et al., Science, 1994, 266(5):243-7, Chang et al., Proc Natl Acad Sci USA, 1994, 91:12544-1248, and U.S. Pat. No. 8,679,711.

### E. Recombinant Protein Expression and Purification

Subtiligase variant polypeptides can be obtained by methods well known in the art for protein purification and recombinant protein expression. Any method known to those of skill in the art for identifying nucleic acids that encode desired genes can be used.

Subtiligase variants can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening. Methods for amplification of nucleic acids can be used to isolate nucleic acid molecules encoding a subtiligase polypeptide, including for example, polymerase chain reaction (PCR) methods. Amino acid substitutions can be introduced into a wild-type or parental subtiligase polypeptide or a wild-type or parental stabiligase polypeptide by, for example, by site-directed mutagenesis.

For recombinant expression of a subtiligase variant, the nucleic acid containing all or a portion of the nucleotide sequence encoding the subtiligase protein can be inserted into an appropriate expression vector, i.e., a vector that contains the necessary elements for the transcription and translation of the inserted protein coding sequence. The resulting expression vector can be introduced into a host cell such as a eukaryotic cell or a prokaryotic cell. Suitable hosts for production of the subtiligase variant are homologous or heterologous hosts, such as the microbial hosts including bacterial cells, yeast cells, and fungal cells, and other host cells including insect cells, plant cells, and mammalian cells. In some cases, the host cell is E. coli. Suitable host cells are discussed herein and further in Goeddel, Gene Expression Technology: Methods in Enzymology 185, Academic Press, San Diego, Calif. (1990) and in “Production of Recombinant Proteins: Novel Microbial and Eukaryotic Expression Systems”, 2004, Wiley-Blackwell.

A variety of host-vector systems can be used to express the protein coding sequence. These include but are not limited to mammalian cell systems infected with virus insect cell systems infected with virus; microorganisms such as yeast containing yeast vectors; or bacteria transformed with bacteriophage, DNA, plasmid DNA, cosmid DNA or the like. The expression elements of vectors vary in their strengths and specificities. Depending on the host-vector system used, any one of a number of suitable transcription and translation elements can be used.

Methods for the production and purification of subtiligase polypeptides from host cells depend on the chosen host cells and expression systems. A suitable culture medium (fermentation medium) may contain sources of carbon and nitrogen besides inorganic salts optionally together with growth promoting nutrients, such as yeast extract that facilitate the production of subtiligase. For secreted molecules, proteins are generally purified from the culture media after removing the cells. For intracellular expression, cells can be lysed and the proteins purified from the extract.

Subtiligase polypeptides according to the invention can be recovered and purified from recombinant cell cultures by methods known in the art (Protein Purification Protocols, Methods in Molecular Biology series by Paul Cutler, Humana Press, 2004).

In some embodiment, an isolated subtiligase variant has a purity of at least about 80% (by dry weight), e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%. In certain embodiments, an isolated subtiligase variant has a purity of about 90% or more, 95% or more, 98% or more, or 99% or more. In some cases, the isolated subtiligase variant is pure or substantially pure.

### F. Kits

The invention provides kits for practicing the methods described herein. Such kits can include a subtiligase variant described herein. In some embodiments, the kit includes a nucleic acid encoding any of the variants, an expression vector comprising any of the nucleic acids and/or a host cell containing any of the nucleic acids or any of the expression vectors. The kit can include a peptide ester substrate or a peptide thioester substrate wherein this substrate includes a payload such as a detectable moiety, therapeutic moiety, chemical moiety, drug moiety, binding moiety, nucleic acid, or reactive group.

The following examples are intended to illustrate exemplary embodiments of the invention are not to be construed as limiting its scope or applications.

### EXAMPLE 1

### Engineering Peptide Ligase Specificity by Proteomic Identification of Ligation Sites

### Abstract

Enzyme-catalyzed peptide ligation is a powerful tool for site-specific protein bioconjugation, but stringent enzyme-substrate specificity limits its utility. Here, we present an approach for comprehensive characterization of peptide ligase specificity for N termini using proteome-derived peptide libraries. We used this strategy to characterize the ligation efficiency for >25,000 enzyme-substrate pairs in the context of the engineered peptide ligase subtiligase and identified a family of 72 mutant subtiligases with activity toward N-terminal sequences that were previously recalcitrant to modification. We applied these mutants individually for site-specific bioconjugation of purified proteins including antibodies, and in algorithmically selected combinations for sequencing of the cellular N-terminome with reduced sequence bias. We also developed a web application to enable algorithmic selection of the most efficient subtiligase variant(s) for bioconjugation to user-defined sequences. These studies provide a new toolbox of enzymes for site-specific protein modification and a general approach for rapidly defining and engineering peptide ligase specificity.

### Introduction

Site-specific protein modification strategies have enabled a wide array of advances in the biological sciences, including development of probes of enzyme function (1-3), discovery of enzyme inhibitors and drugs (4-6), synthesis of antibody-drug conjugates (7, 8), and implementation of advanced imaging techniques (9). Site-specific strategies include modification of engineered cysteine or methionine residues (2, 7, 10), enzymatic ligation to genetically encoded sequence epitopes (11-15), introduction of unnatural amino acids (16-18), and native chemical ligation (19-21). Although these methods have proven powerful for a number of applications, they require genetic engineering of the protein of interest, which may disrupt biological function, reduce expression yield, and limit their utility as chemoproteomic probes

The N-terminus is a universal feature of all proteins that is an attractive handle for site-specific protein modification based on its uniqueness within each polypeptide chain (22-24). Although a number of chemical strategies target the N terminus (25-31), they are commonly limited by poor selectivity, the requirement for particular N-terminal residues, or the inability to form a native peptide bond. Because of its ability to target the N-terminus and to generate a native peptide bond, enzyme-catalyzed peptide ligation is an attractive alternative strategy for protein modification (32). Early efforts focused on using proteases in reverse to achieve peptide ligation, but these often suffered from low yields, high reversibility, and harsh reaction conditions (33-35).

More recently, naturally occurring peptide ligases, which catalyze transpeptidation reactions, have been applied to target the N terminus for protein bioconjugation. These include the bacterial cell wall biosynthesis enzyme sortase (11) and the cyclotide macrocyclization enzyme butelase 1 from Clitoria ternatea (36). However, these enzymes retain strict sequence requirements programmed from their native biological functions, often creating the need to genetically engineer the target ligation site. This results in non-native ‘scars’ in the target protein either C-terminal to and/or N-terminal to the newly formed amide bond, P′ or non-prime P, respectively according to Schechter and Berger nomenclature (37). In contrast, the engineered peptide ligase subtiligase, which catalyzes a ligation reaction between a peptide ester or thioester and the N-terminal α-amine of a protein or peptide (38) (FIG. 1A), has broader specificity and higher catalytic efficiency (>10⁵ M⁻¹ s⁻¹) compared to sortase (11, 39, 40) or butelase 1 (36). Moreover, subtiligase can be expressed at high levels in Bacillus subtilis, making it easy to generate and purify recombinant variants (41). However, qualitative specificity studies show that this enzyme harbors narrowed sequence specificity that limits its utility for N-terminal bioconjugation, block-wise synthesis of proteins, and N-terminomics studies (42, 43). Furthermore, incomplete characterization of its N-terminal specificity makes the suitability of subtiligase for any particular application difficult to predict.

Presented herein is a strategy for comprehensive characterization of peptide ligase prime-side specificity that utilizes database-searchable, proteome-derived peptide libraries as ligase substrates. Inspired by a method for mapping protease sequence specificity (44), this approach enables selective isolation of ligated peptides and sequencing by liquid chromatography-tandem mass spectrometry (LC-MS/MS) for rapid determination of positional enrichment or de-enrichment of each amino acid at each P′ site. This approach, termed Proteomic Identification of Ligation Sites (PILS), was applied to more comprehensively characterize subtiligase prime-side specificity, and combined it with alanine scanning mutagenesis at 20 sites to systematically identify sites that affect substrate sequence selectivity. PILS was deployed to screen single-site saturation mutagenesis libraries at the most impactful sites, enabling identification of a panel of variants that redirect subtiligase activity toward sequences that were previously refractory to modification. The utility of these variants individually for bioconjugation of diverse payloads to purified proteins, and in algorithmically selected cocktails to achieve more comprehensive sequencing of the cellular N-terminome in bacterial and human cells was demonstrated. Also, a web application is described to enable users to leverage the extensive PILS data that was collected to algorithmically select the most efficient subtiligase variants for labeling particular sequences or groups of sequences, and to analyze additional PILS datasets. These results establish a family of peptide ligases with defined specificities, and greatly expand the toolbox of enzymes available for site-specific modification of protein N termini. Additionally, they provide a platform for rapid characterization and engineering of ligase specificity.

### Results

### Proteome-Derived Peptide Libraries to Map Ligase Specificity.

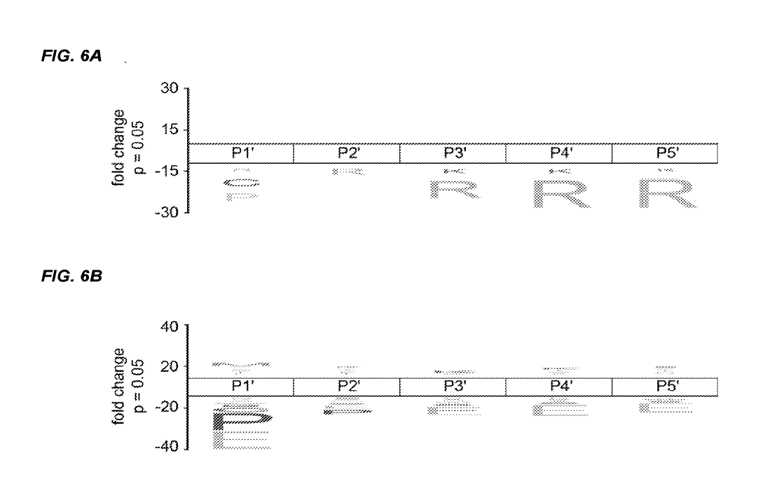

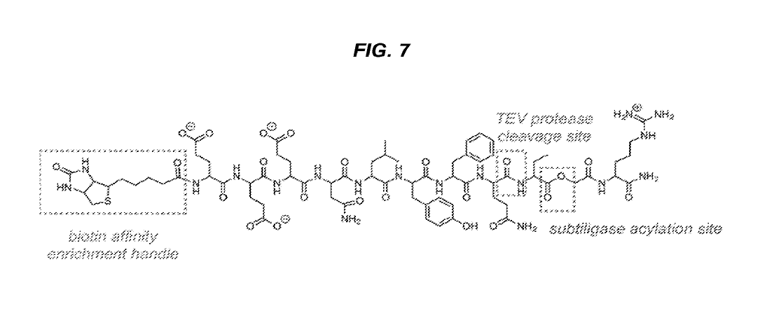

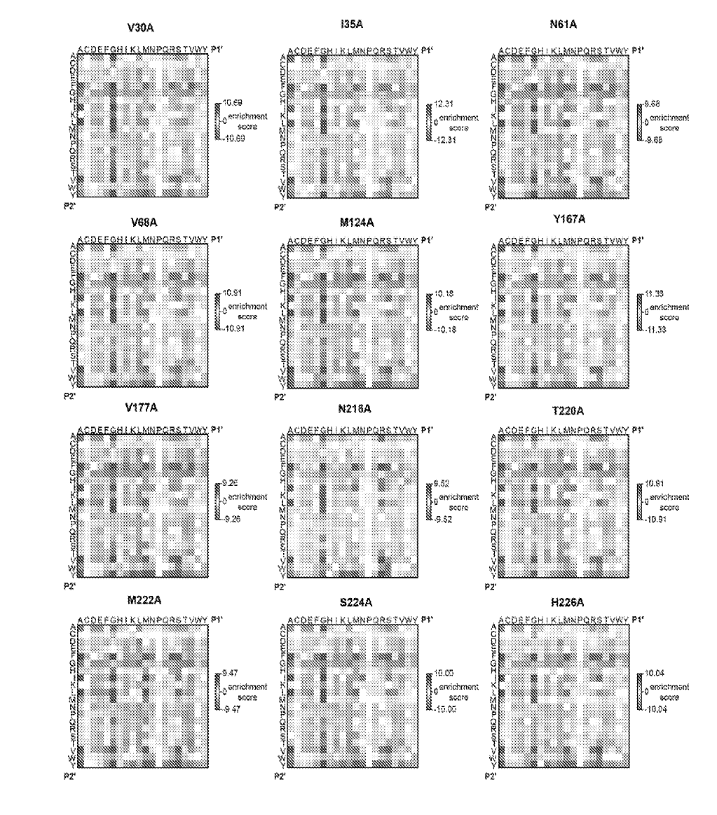

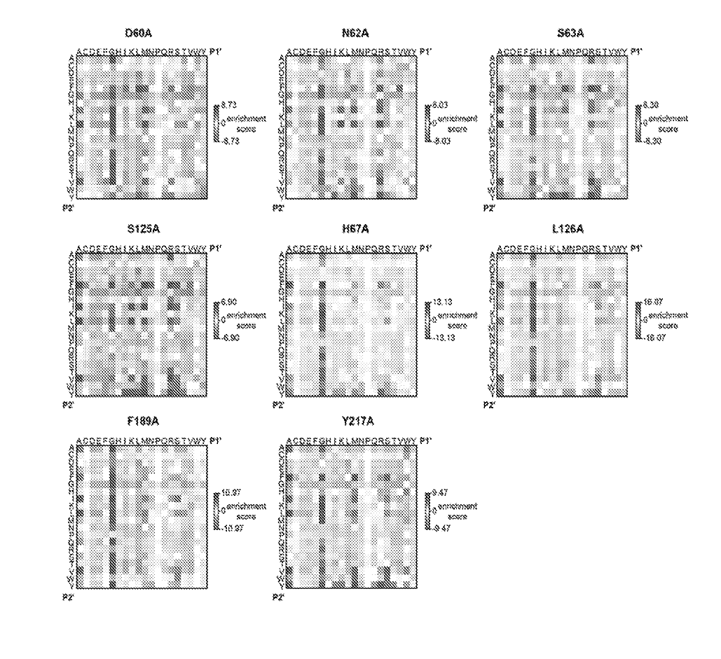

To enable comprehensive characterization of subtiligase prime-side specificity, we developed a mass spectrometry-based assay, PILS, inspired by the Proteomic Identification of protease Cleavage Sites (PICS) method for determining protease specificity (44). Diverse α-amine acceptor peptide libraries was generated by digesting the E. coli proteome with two proteases of orthogonal P1 specificity: trypsin, (P1=K or R) or Glu-C (P1=E or D). This produced two libraries that, in combination, represent every possible single amino acid from P1′-P6′, and nearly all 400 P1′-P2′ dipeptide combinations. Proline was underrepresented compared to natural abundance based on the inability of the digest proteases to cleave sequences with P1′ proline, while cysteine was underrepresented and modified by carbamidomethylation to prevent peptide crosslinking, precluding analysis of specificity for the natural cysteine side chain (FIGS. 6A-6B). Each acceptor peptide library was incubated with subtiligase (1 μM) and a limiting amount of the donor peptide ester 1 (200 μM, FIG. 7) that contains an N-terminal biotin for avidin capture, a TEV protease cleavage site for complete and unbiased (45, 46) proteolytic release, and an aminobutyric acid (Abu) mass tag for unequivocal confirmation that ligation occurred (47, 48; FIG. 1B). Both the unenriched input libraries and the enriched, eluted peptides were analyzed by LC-MS/MS to quantify the frequency with which each amino acid appeared in each position. In each enriched sample, >2,000 Abu-tagged subtiligase substrate peptides were identified (Supplementary Dataset 1).

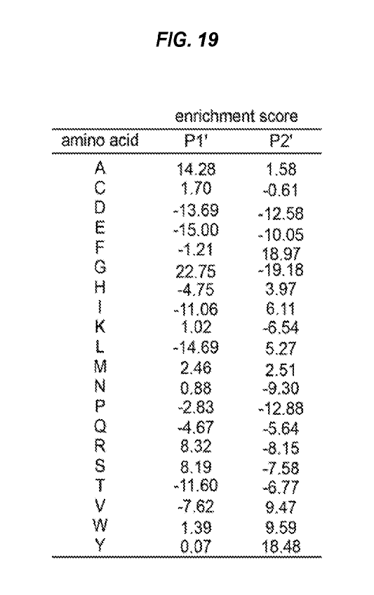

To determine subtiligase sequence specificity, the position-specific differences in the frequencies of each amino acid in enriched samples compared to the input libraries was evaluated by calculating an enrichment score (see Methods). No significant sequence specificity beyond P2′ of the ligated peptide was observed based on lack of enrichment, consistent with previous structural studies of subtilisin (FIG. 1C, FIG.19). However, significant sequence preferences in both the P1′ and P2′ positions was observed. Small amino acids (Ala, Gly, and Ser), Met, and Arg were significantly enriched at P1′, while acidic residues (Asp and Glu), branched-chain amino acids (Ile, Leu, Thr, and Val), Pro, and Gln were significantly de-enriched. At P2′, aromatic (Phe, Trp, and Tyr) and large hydrophobic (Ile, Leu, and Val) residues were significantly enriched, while acidic, basic, and polar residues (Asn, Gln, and Ser), Gly, and Pro were significantly de-enriched. These results are in general agreement with previous qualitative studies of subtiligase specificity (42), as well as with quantitative studies of a small number of individual peptides (38), demonstrating the validity of the PILS method for determining ligase specificity.

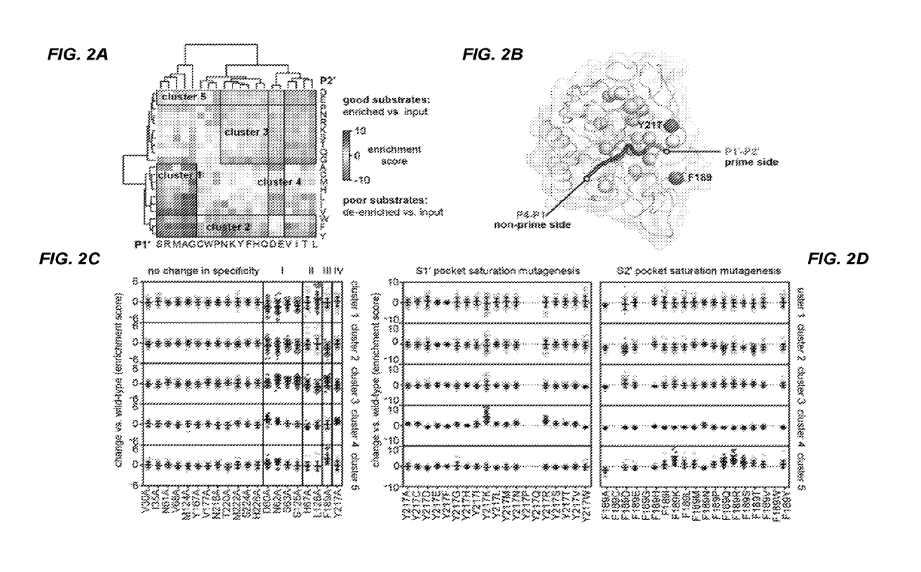

It is well known that substrate amino acid subsites in proteases exhibit cooperativity that cannot be assessed by evaluating one position at time (49). To examine the role of subsite (S) cooperativity in sequence recognition at P1′ and P2′, the enrichment or de-enrichment of each dipeptide sequence relative to the input library was measured. Hierarchical clustering on the enrichment scores was performed and identified five clusters of dipeptide sequences that behave similarly to one another (FIG. 2A, Supplementary Dataset 1). Sequences in cluster 1 are good substrates for subtiligase and have a small amino acid, Met or Arg in the P1′ position and an aromatic or large hydrophobic, His, Met, Cys, or Ala in the P2′ position. Sequences in cluster 2 are also good substrates for subtiligase, and have any amino acid except Asp or Glu at P1′ and an aromatic amino acid at P2′. Sequences in cluster 3 are poor substrates for subtiligase and contain aromatic, large hydrophobic, polar, or acidic residues at P1′, and acidic, basic, or polar residues at P2′. Sequences in clusters 4 and 5 are also poor substrates for subtiligase and contain acidic residues at P1′ and P2′, respectively.

Subsite cooperativity is apparent based on examination of the dipeptide heatmap (FIG. 2A). For example, when P2′ is a favorable aromatic residue, any amino acid except Asp or Glu is accepted in the P1′ position. Similarly, when a favorable amino acid is present in the P1′ position, a broader set of amino acids are accepted at P2′. These results suggest that energetically favorable interactions at one subsite can help to overcome weak or unfavorable interactions at the other subsite, expanding the total number of sequences that can be efficiently ligated by subtiligase. These insights would not have been possible with other approaches for quantifying ligase specificity, such as positional scanning combinatorial peptide libraries, which only map one position at a time in a fixed context.

Defining the SI' and S2′ Subsites through Alanine Scanning Mutagenesis

While the S1-S4 pockets of subtilisin have been defined through extensive structural, biochemical, and mutagenesis studies (50-52), the S1′ and S2′ pockets are poorly understood. Because protease specificity is sometimes determined by elements that are not structural components of the substrate binding site (53), a functional approach was chosen to determine key residues for substrate recognition. As a starting point, wild-type subtiligase (subtilisin-S221C/P225A) was chosen, as mutations that have been introduced subsequently to improve subtiligase stability have not been characterized with respect to their impact on substrate specificity. To systematically determine which residues functionally contribute to substrate binding, alanine-scanning mutagenesis was performed on twenty residues within 7 Å of the catalytic triad of subtiligase, confirming the identity of each mutant by Sanger sequencing (FIG. 2B). These mutants were purified and their identities were verified by mass spectrometry (FIG. 20A, FIG. 20B). The resultant changes in ligation specificity were quantified using the PILS method (FIG. 2C, FIG. 8A, FIG. 8B, Supplementary Datasets 2-21). Residues were interpreted to contribute significantly to prime-side specificity if the corresponding alanine mutation led to a change in the distribution of Abu-labeled peptides in any of the five previously defined sequence clusters by at least two standard deviations compared to the wild-type enzyme. This corresponded to an enrichment score of ≥2 or ≤−2 when the wild-type data is used as the reference set. Of the twenty alanine mutants studied, twelve did not significantly change the observed frequency of labeling in any of the five sequence clusters. The remaining eight mutants showed significant changes in specificity and could be divided into four classes based on similarities in their patterns of specificity change.

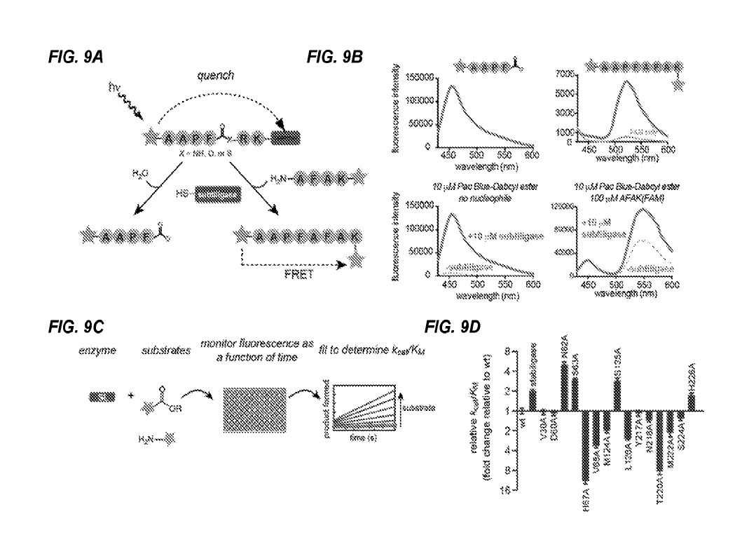

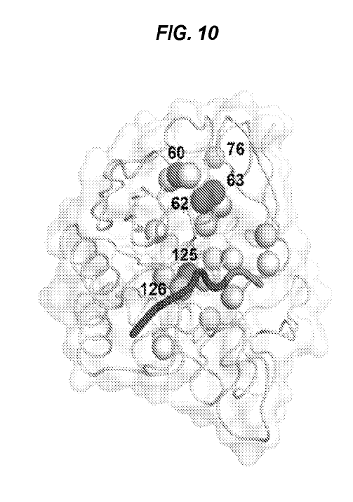

Class I mutants (D60A, N62A, S63A, and S125A) are characterized by enhanced labeling of poor substrates (clusters 3, 4, and 5) and decreased labeling of good substrates (clusters 1 and 2), representing apparent broadened specificity (FIG. 2C, FIG. 8A, FIG. 8B). Conversely, class II mutants (H76A and L126A) show the opposite pattern. Kinetic analysis of these mutants revealed that class I mutations increase k_{cat}/K_{M} by 4-8-fold compared to the wild-type enzyme, while class II mutations decrease k_{cat}/K_{M} by 4-16-fold (FIGS. 9A-9D). The apparent changes in specificity therefore likely result from catalytic differences rather than increased or decreased molecular recognition of particular substrate sequences. Consistent with this conclusion, a structure of subtilisin bound to the Streptomyces subtilisin inhibitor (SSI) (52) suggests that these residues are far from the site in which the prime side of an acceptor peptide substrate is expected to bind (FIG. 2B, FIG. 10). Therefore, although some of these mutants are useful for enhancing subtiligase's catalytic efficiency, class I and class II residues do not functionally contribute to binding of P1′ and P2′ substrate residues.

Class III includes only one mutant, F189A, which is characterized by a decrease in modification of cluster 2 sequences and an increase in modification of cluster 3 and cluster 5 sequences (FIG. 2C, FIG. 8A, FIG. 8B). The identity of the P2′ residue defines both cluster 2 (P2′=aromatic) and cluster 5 (P2′=acidic), and contributes to cluster 3 (P2′=charged, polar, Pro, or Gly), suggesting that Phe 189 is a major determinant of P2′ specificity and makes up part of the S2′ pocket. Similarly, class IV only includes one mutant, Y217A, and is characterized by an increase in modification of cluster 4 sequences and a decrease in labeling of cluster 3 sequences (FIG. 2C). Cluster 4 is defined by the identity of the P1′ residue (P1′=acidic), suggesting that Tyr 217 is an important determinant of P1′ specificity. Consistent with these conclusions, both Phe 189 and Tyr 217 lie near the predicted prime side interaction site based on the subtilisin-SSI structure (52). Although other nearby residues, such as Asn 218 and Thr 220, may have been predicted to impact substrate recognition based on structure alone, no significant differences in substrate specificity were observed when these residues were mutated to alanine. These results suggest that while structural inspection is useful for targeting sites, it is not a substitute for systematic mutagenesis and PILS to ensure completeness of the analysis.

Engineering Subtiligase Variants with Altered Specificity

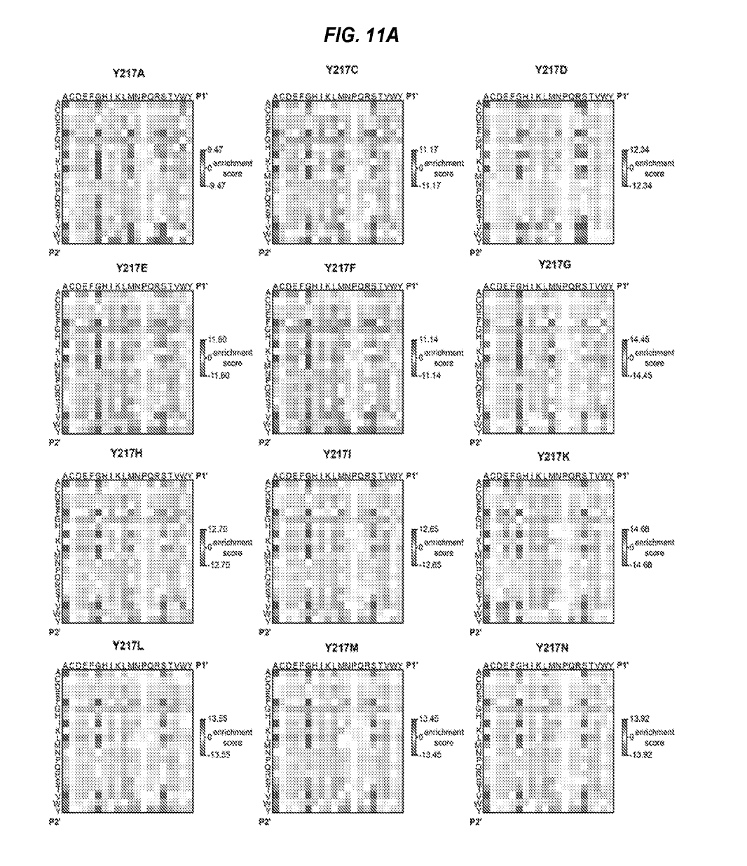

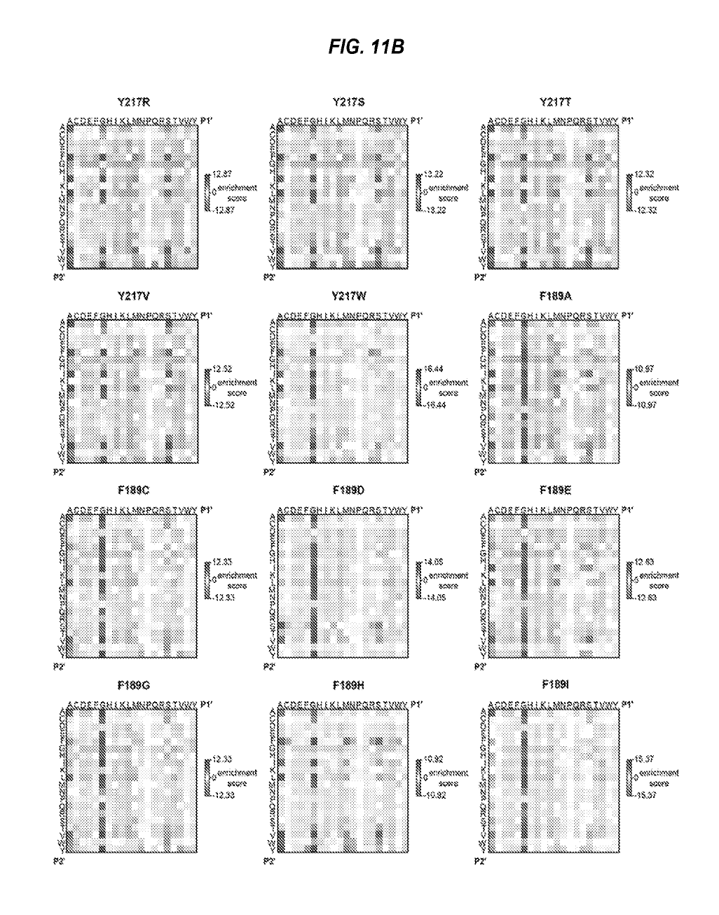

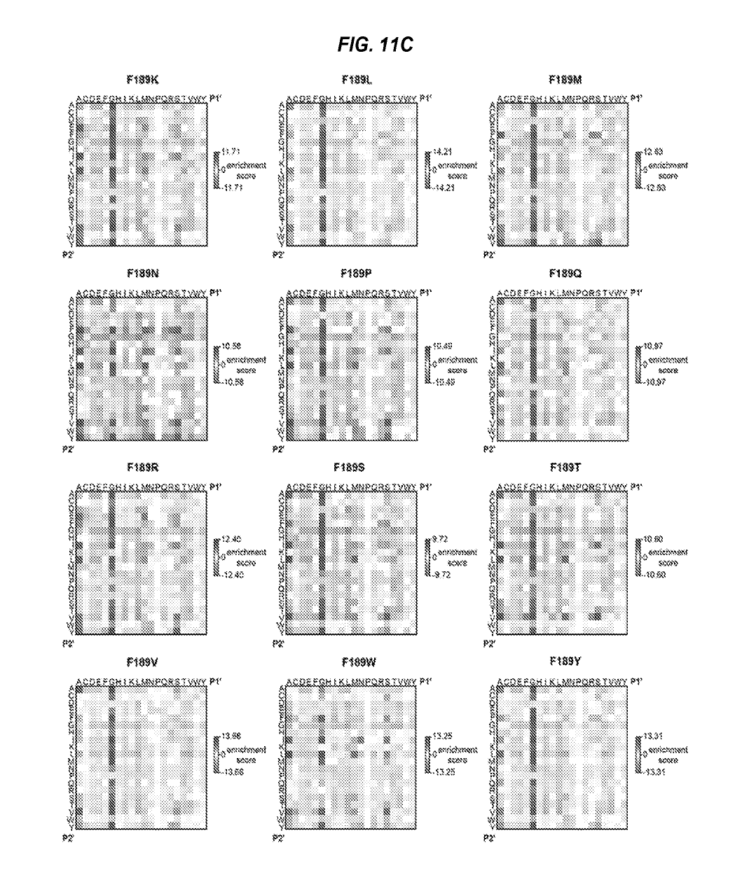

Having identified key residues in the 51′ and S2′ pockets, we set out to engineer subtiligase variants with altered or broadened specificity. The initial approach was to perform saturation mutagenesis at positions 189 (S2′ pocket) and 217 (S1′ pocket) of subtiligase. To determine how each mutation impacted subtiligase specificity, we purified the 36 mutants and analyzed their specificity profiles using the PILS method. Mutations that led to a change in the distribution of labeled peptides in at least one of the five dipeptide sequence clusters of >2 standard deviations were considered to have a significant impact on specificity. Based on the functional analysis above, we targeted ‘hot spot’ positions 189 (S2′ pocket) and 217 (S1′ pocket) for saturation mutagenesis to fully explore how these positions impact specificity. To determine how each mutation influenced subtiligase specificity, we purified the 36 additional single mutants (expression yields varied between 20-100 mg/L, FIG. 20A, FIG. 20B) and analyzed their specificity profiles using PILS (FIG. 2D, FIG. 11A-FIG. 11C, Supplementary Datasets 22-52). At position 217, the Y217K and Y217R mutations led to significant improvements in modification of sequences with an acidic P1′ residue relative to both the wild-type enzyme and the Y217A mutant. Providing validation of our screening strategy, the Y217K mutant was rationally incorporated into subtiligase previously to enable Cys-free, enzyme catalyzed expressed protein ligation in the context of acidic P1′ sequences (21). The Y217D and Y217E mutants showed improved modification of peptides containing P1′ His, Lys, Ser, or Arg. At position 189, the F189S, F189Q, F189K, and F189R mutations led to significant improvement in modification of sequences with an acidic P2′ residue and diminished labeling of sequences with an aromatic residue at P2′. Additionally, although they did not shift specificity on average across an entire cluster, a number of other mutations led to improved or diminished modification of specific sequences (FIG. 11A-FIG. 11C). Together, these results indicated that introduction of a charged or polar residue in either the S1′ or S2′ pocket creates the opportunity for new, favorable electrostatic or hydrogen bonding interactions with charged or polar peptide substrates, expanding the number of N-terminal dipeptide sequences that can be efficiently labeled with subtiligase. These mutants and the accompanying PILS specificity maps (FIG. 11A-FIG. 11C) represent a new toolbox of peptide ligases that can be deployed for a variety of applications based on their specific N-terminal specificity requirements.

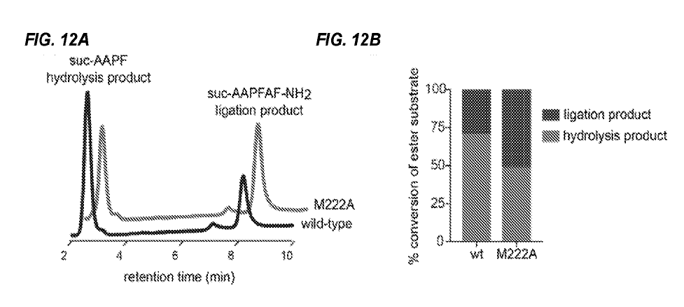

While a number of F189 mutants showed useful changes in sequence specificity, many of these mutants expressed at much lower levels compared to wild-type subtiligase. Furthermore, when we characterized these mutants by LC-MS, we observed a 16 Da mass modification consistent with methionine oxidation (FIG. 20A, FIG. 20B). Previous studies of subtilisin demonstrated that Met 222 is prone to an oxidation event that impacts enzyme activity (54), and protein engineering work determined that substitution of alanine or glycine at this position improves subtilisin activity (55) and enhances the aminolysis-to-hydrolysis ratio in the context of subtiligase (56). Tthe F189 and Y217 mutants with significant specificity changes were introduced into the more stable heptamutant of subtiligase (M50F, N76D, N109S, M124L, S125A, K213R, N218S) termed stabiligase (42), or an octamutant that also incorporated the M222A mutation. To enable one-step affinity purification of subtiligase, stabiligase, and mutants, a C-terminal His₆ tag was also introduced. These mutants expressed at levels comparable to wild-type subtiligase and maintained specificity profiles indistinguishable from the mutants in the subtiligase background (FIG. 11A-FIG. 11C, Supplementary Datasets 53-72). It was found that introduction of the M222A mutation both eliminated the observed oxidation event and improved the subtiligase peptide ligation to hydrolysis ratio (FIG. 20A, FIG. 20B, FIG. 12A, FIG. 12B), consistent with previous studies. To further enable adoption of subtiligase-catalyzed bioconjugation, a pro domain- and Ca²⁺-independent variant suitable for expression in E. coli and one-step affinity purification was also constructed. This variant was purified in high yield (˜20 mg/L) and, in the context of the Y217K mutant, exhibited a specificity profile similar to subtiligase-Y217K and stabiligase-Y217K (FIG. 11A-FIG. 11C, Supplementary Dataset 73).

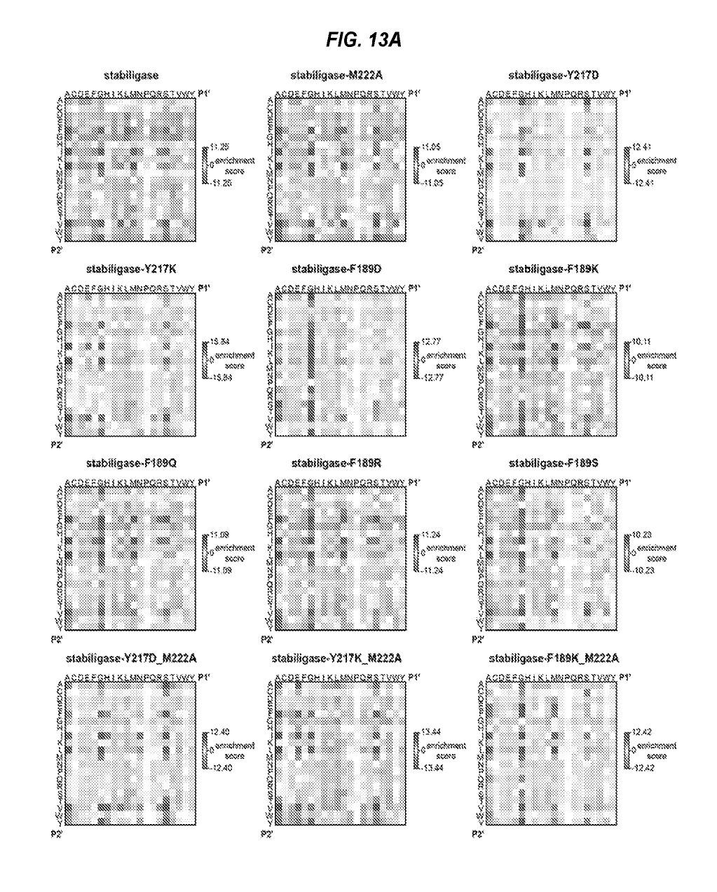

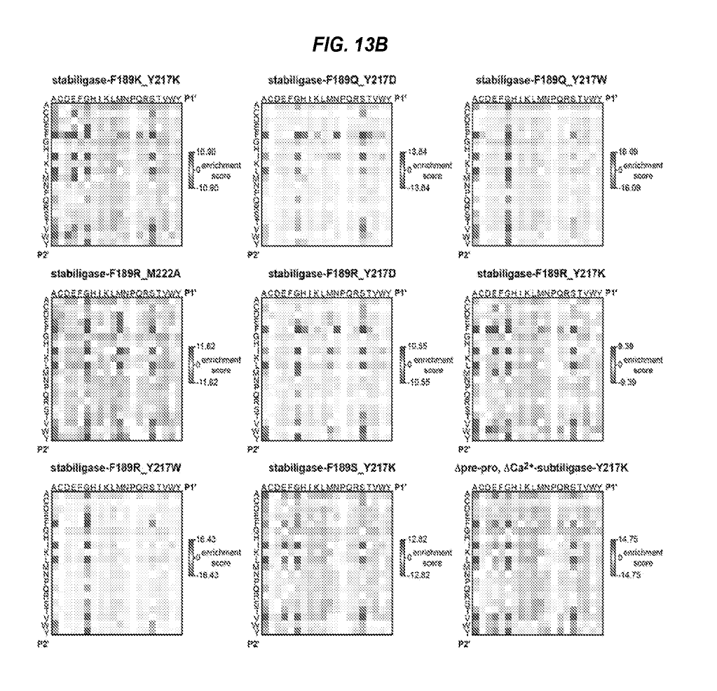

In the context of stabiligase, the specificity of a number of F189/Y217 double mutants, including F189K/Y217K, F189R/Y217K, F189R/Y217D, F189S/Y217K, and F189Q/Y217D were examined (FIG. 13A, FIG. 13B, Supplementary Datasets 61-72). The double mutants impacted sequence specificity in a predictable way based on PILS analysis of the single mutants, suggesting that our PILS specificity datasets can be leveraged for the design of tailor-made mutants to label specific N-terminal sequences.

Scope of Native Proteins that can be Modified with Subtiligase-Catalyzed Bioconjugation

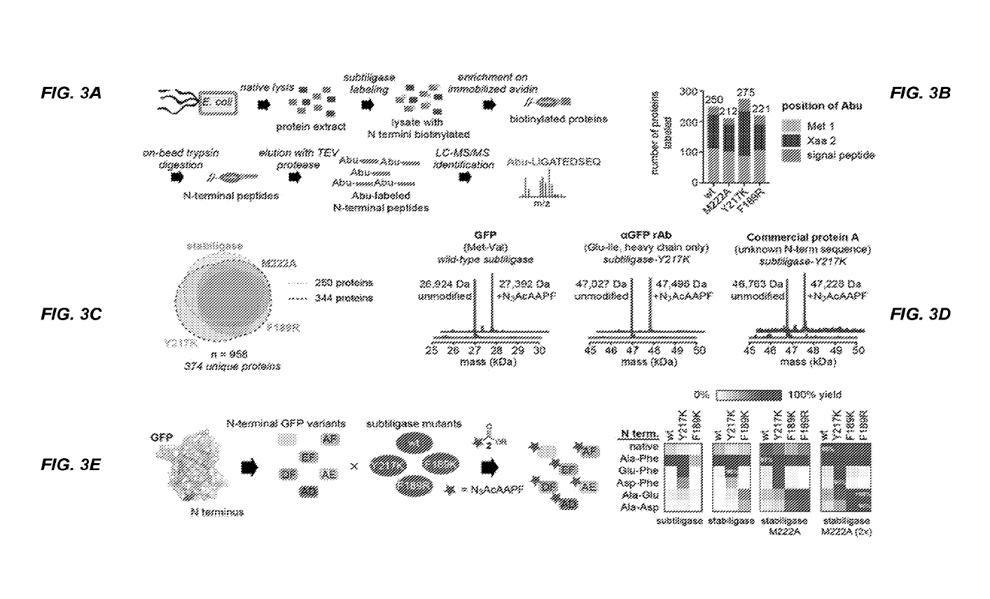

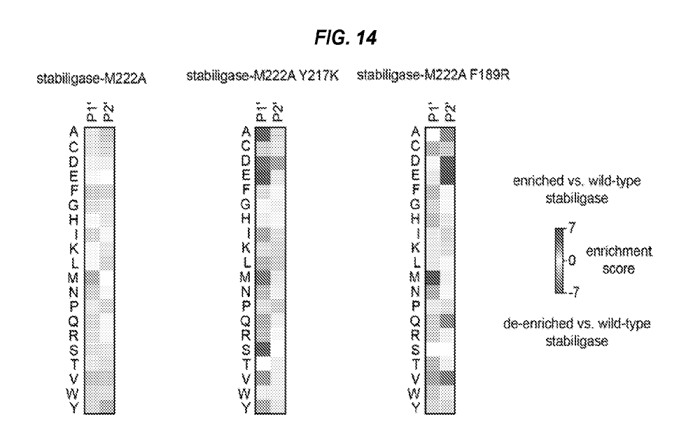

Based on the PILS specificity maps that were generated, it was hypothesized that the panel of mutants that we characterized would expand the number of N-terminal protein sequences that can be targeted for site-specific protein bioconjugation by subtiligase. As an initial test of this hypothesis, E. coli lysate were generated under native conditions and incubated it with wild-type stabiligase, stabiligase-M222A, stabiligase-Y217K/M222A, or stabiligase-F189R/M222A and biotinylated peptide ester 1 (FIG. 3A). Following labeling, the biotinylated proteins were isolated on immobilized neutravidin, digested with trypsin to remove internal peptides, selectively eluted the Abu-tagged N-terminal peptides with TEV protease, and sequenced them by LC-MS/MS. Each subtiligase variant labeled >200 native proteins at their translational N-termini or at annotated signal peptide cleavage sites (FIG. 3B). Compared to stabiligase alone, the mutants increased the number of native proteins that could be labeled by 50%, from 250 to 374, with the N-terminal sequences of the additional proteins reflecting the altered specificity of the mutants as measured by PILS (FIG. 3C, FIG. 14, Supplementary Dataset 74). These results demonstrate the ability of engineered subtiligase mutants to modify a broader swath of N-terminal sequence space without requiring genetic modification of the target protein.

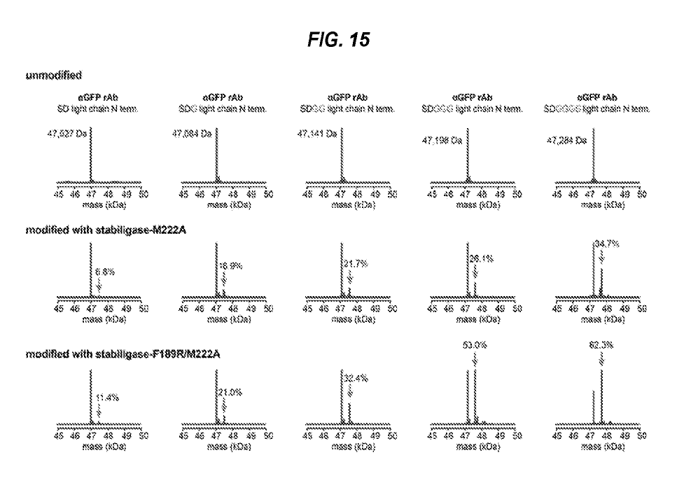

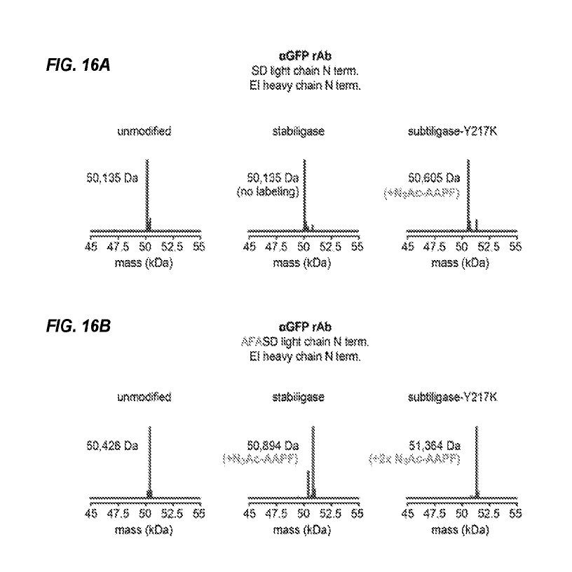

To examine the utility of engineered subtiligase mutants for high-yield protein bioconjugation, the ability of the engineered mutants to modify recombinant antibodies, an important class of therapeutic proteins (FIG. 3D) was tested. The inventors, as part of the Recombinant Antibody Network (RAN), have produced an automation platform for producing thousands of recombinant antibodies to more than 500 protein targets using a synthetic Fab library displayed on filamentous phage (57). Because all of these antibodies are built on a single scaffold derived from Trastuzamab (58, 59), they have a common N-terminal light chain sequence of Ser-Asp and a common N-terminal heavy chain sequence of Glu-Ile. Based on PILS analysis of wild-type subtiligase, both of these N-terminal sequences are predicted to be poor ligation substrates. To test this prediction, we attempted to ligate azide-bearing peptide ester 2 onto the N-terminus of an anti-GFP antibody (αGFP) that we constructed (60). Indeed, αGFP was completely refractory to modification with wild-type subtiligase (FIG. 15). Based on the PILS specificity maps, we predicted that αGFP could be labeled on the heavy chain by the Y217K mutant, and indeed, we observed quantitative labeling on the heavy chain by subtiligase-Y217K within 1 h (FIG. 3D), demonstrating that high-yield bioconjugation can be achieved by judicious matching of enzyme and substrate. PILS also predicted that the Ser-Asp N terminus of the light chain would be labeled specifically by the stabiligase-F189R/M222A mutant having favorable specificity for P2′ Asp. However, no significant labeling was observed within 1 h in this context, while overnight incubation produced the peptide-antibody bioconjugate in 11.4% yield (FIG. 15). It was hypothesized that the inefficiency of light chain modification was due to inaccessibility of the N-terminus, a limitation that has been demonstrated to impact yield from other N-terminal modification methods (25). To test this hypothesis, the light chain N terminus was extended by one (Gly), two (Gly-Gly), three (Gly-Gly-Gly), or four (Gly-Gly-Gly-Ser; SEQ ID NO: 21) residues while maintaining the native N-terminal sequence. Indeed, we observed increased modification yields of 21% (Gly), 32% (Gly-Gly), 53% (Gly-Gly-Gly), and 62% (Gly-Gly-Gly-Ser; SEQ ID NO: 21) (FIG. 15). These results suggest that inefficiency due to N-terminal inaccessibility can be overcome by multiple rounds of labeling when genetic modification is not an option, or by modification of the N terminus to enhance its accessibility.

The study next set out to test whether orthogonality could be achieved in the context of the αGFP heterodimer. The N terminus was extended by three residues (Ala-Phe-Ala) having a favorable sequence for wild-type subtiligase. Within 1 h, specific and quantitative labeling of the light chain only with wild-type subtiligase and labeling of both the heavy and light chains with subtiligase-Y217K (FIG. 16A, FIG. 16B) was observed. These results demonstrate that N-terminal inaccessibility can be overcome with short N-terminal extensions, and that careful selection of subtiligase mutants matched to their optimal substrates by PILS can produce specific and orthogonal labeling in the context of heterodimers or protein mixtures.

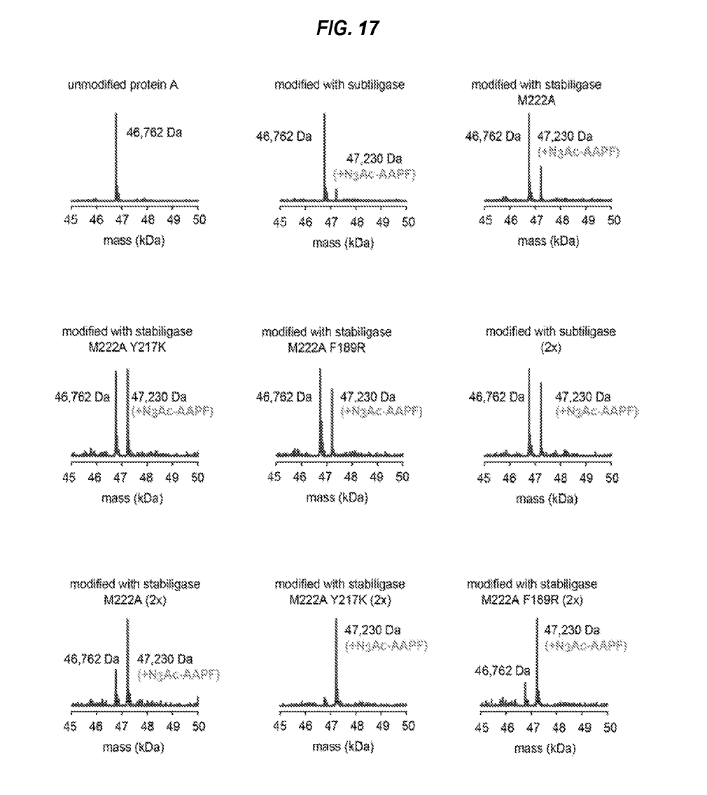

When the N-terminal sequence of the target protein is known, the PILS data that was collected enables it to be matched with a subtiligase variant that will label that sequence efficiently. Next it was asked whether a small panel of subtiligase mutants covering a broad swath of sequence space could label a protein without knowledge of the N-terminal target sequence. To test this, engineered recombinant protein A, whose N-terminal sequence was unknown to the experimenters was purchased. A panel of five stabiligase mutants were tested and it was discovered that one mutant, Y217K, could indeed label protein A quantitatively (FIG. 3D, FIG. 17), demonstrating the feasibility of subtiligase modification even in the absence of sequence information.

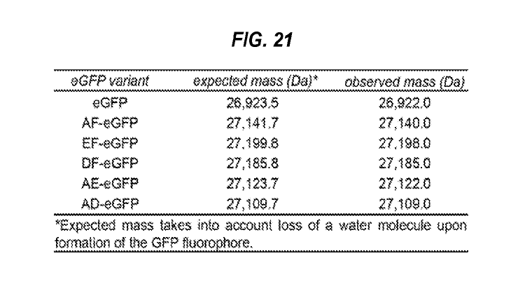

The labeling yield produced by engineered subtiligase was tested using green fluorescent protein (GFP) with its native N terminus (Met-Val), or engineered N termini that were either good (Ala-Phe) or poor (Glu-Phe, Asp-Phe, Ala-Glu, and Ala-Asp) substrates for wild-type subtiligase as predicted by PILS specificity maps (FIG. 21). These GFP variants were modified by the azide-bearing peptide ester 2 and a panel of ten subtiligase mutants: Y217K, F189K, and F189R in the context of wild-type subtiligase (FIG. 3E, left panel), stabiligase (FIG. 3E, middle-left panel), or stabiligase-M222A (FIG. 3E, middle-right panel). Because subtiligase retains esterase activity, it was reasoned that ligation yields might be improved by removing cleaved substrate and adding of a second batch of substrate. Therefore, a fourth set of reaction conditions was included in which the GFP N-terminal variants were labeled a second time following desalting of the reaction mixture (FIG. 3E, right panel). As predicted by PILS, Ala-Phe-GFP could be labeled nearly quantitatively with wild-type subtiligase, stabiligase, and stabiligase-M222A (FIG. 3E, second row) . In contrast, the labeling yield for the remaining sequences was poor (<25%) when subtiligases retaining wild-type specificity were used. However, Glu-Phe-GFP and Asp-Phe-GFP could be labeled much more efficiently with variants harboring the Y217K mutation (FIG. 3E, third and fourth rows). For Glu-Phe-GFP, >90% bioconjugation yield was achieved by subtiligase-Y217K, stabiligase-Y217K, and stabiligase-M222A/Y217K, while for Asp-Phe-GFP, 95% yield was achieved after two rounds of labeling with stabiligase-M222A/Y217K. In contrast, all other subtiligase variants gave <10% yield with this sequence. Similarly, for Ala-Glu-GFP and Ala-Asp-GFP, ≥90% yield was achieved after two rounds of labeling with stabiligase-M222A/F189K and stabiligase-M222A/F189R. Although the native GFP N terminus is predicted to be a good substrate for wild-type subtiligase, labeling yields were poor with subtiligases retaining wild-type specificity (FIG. 3E, top row). However, >95% yield could be achieved when two rounds of labeling with stabiligase-M222A were performed. Because native GFP is two residues shorter than the other variants tested, this suggested that N-terminal accessibility could be a limiting factor in subtiligase labeling yields. The data suggest that poor N-terminal accessibility can be overcome with multiple rounds of labeling or introduction of a short N-terminal extension.

Scope of Native Proteins that can be Modified with Subtiligase-Catalyzed Bioconjugation

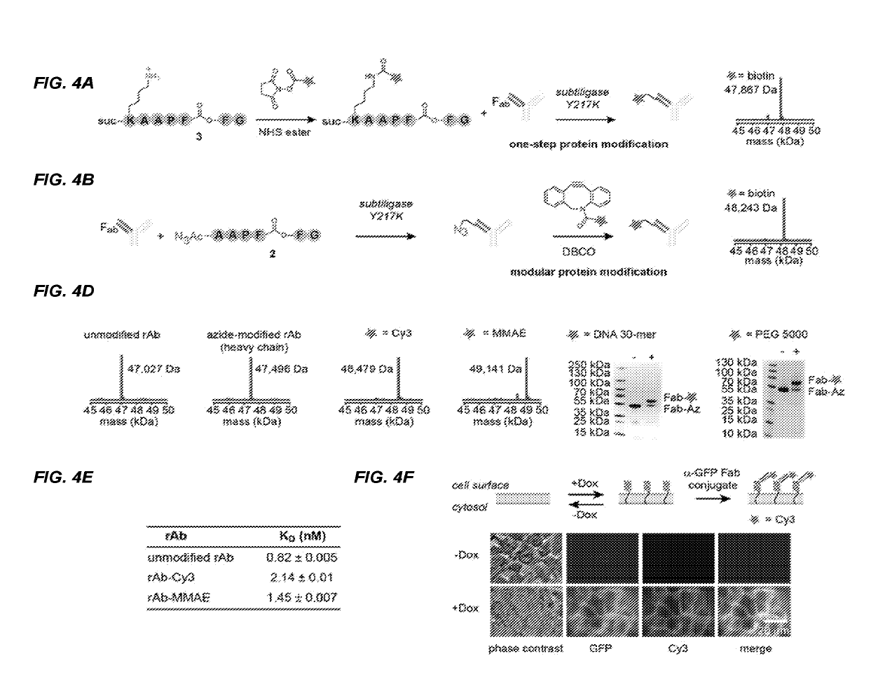

The inventors next set out to develop reagents and protocols for both one-step and modular bioconjugation of diverse payloads to protein N termini using subtiligase. To enable one-step protein modification, an N-terminally capped (succinylated) peptide containing a single free lysine at the subtiligase P5 position, outside the substrate recognition sequence (Compound 3, FIG. 4A) was designed. This free amine was readily acylated with a commercially available biotin N-hydroxysuccinimide ester (NHS ester), enabling site-specific biotinylation with a typically non-specific reagent that would normally target all surface lysines in a protein (FIG. 4A). Numerous NHS ester reagents are commercially available and can be converted to site-specific reagents using the strategy, making this a versatile approach for one-step modification of proteins with diverse payloads.

A modular bioconjugation protocol was also developed by using subtiligase to incorporate a bioorthogonal azide group at the protein N terminus (Compound 2, FIG. 4B). This azide can be modified after incorporation into the protein by copper-catalyzed or copper-free azide-alkyne click chemistry with commercially available alkynes or dibenzyocyclooctynes (DBCOs) or by Bertozzi-Staudinger ligation (61) with commercially available phosphine reagents. Using this modular strategy, we incorporated an azide was incorporated into αGFP and then used this as a starting material for modification with a number of DBCO reagents to produce biotinylated αGFP (DBCO biotin) (FIG. 4B), fluorescent αGFP (DBCO-Cy3), a αGFP-drug conjugate (DBCO-monomethyl auristatin E), an oligonucleotide-modified αGFP (5′-DBCO-oligonucleotide), and a PEGylated αGFP (DBCO-PEG 5000) (FIG. 4C). Importantly, these modifications led to only small decreases in affinity of the αGFP for GFP, demonstrating that protein function is maintained upon modification (FIG. 4D).

To test the utility of these conjugates in a biological context, a HEK-293T cell line modified for doxycycline (Dox)-inducible expression of cell surface GFP in combination with Cy3-αGFP (FIG. 4E) was employed. In Dox induced cells, binding of the Cy3-αGFP and co-localization of the Cy3 and GFP signals were observed. In contrast, no Cy3-αGFP binding was observed in un-induced cells. These results demonstrate the utility of subtiligase-catalyzed N-terminal modification for incorporating probes into proteins while maintaining their biological functions.

### Subtiligase Cocktails for Enhanced Sequence Coverage of the Cellular N-Terminome

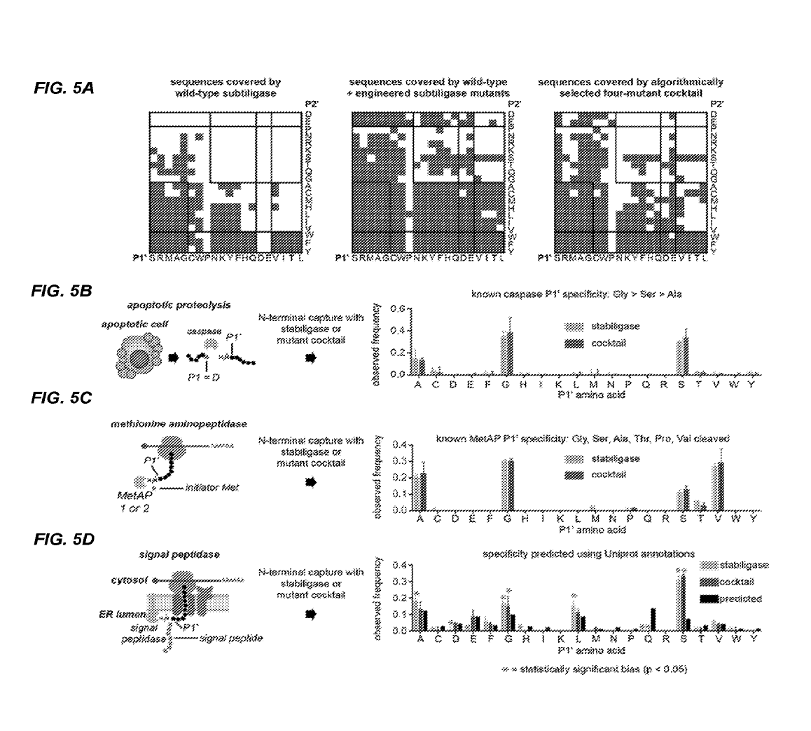

Previously, the inventors had applied subtiligase as a tool for enrichment of proteolytic neo-N termini in the context of apoptotic proteolysis catalyzed by caspases (47, 48, 62). Caspase P1′-P2′ specificity fortuitously encompasses the most preferred sequences for subtiligase ligation based on the results of our PILS studies (63) (FIG. 5A). However, many other proteases have different P1′-P2′ specificity. It was hypothesized that applying cocktails of subtiligase mutants for enrichment of neo-N termini generated by these proteases would more comprehensively capture their prime-side sequence specificity. To test this hypothesis, substrates of two proteases were selected for analysis, methionine aminopeptidase (MetAP) and signal peptide peptidase (SPP), which have divergent specificities (64, 65). For comparison, we also analyzed apoptotic proteolysis (FIG. 5B) was also analyzed. Using the PILS datasets, three stabiligase mutants, F189S/Y217K, F189D, and Y217D/M222A, that are capable of modifying the maximum number of N-terminal dipeptide sequences with an enrichment score ≥0 in combination with wild-type stabiligase were algorithmically selected. A Jurkat cell lysate were labeled with 2.5 mM biotinylated ester 1 and either a mixture of 1 μM of each mutant (4 μM total enzyme) or 4 μM stabiligase. Biotinylated proteins were enriched on immobilized neutravidin and digested with trypsin to isolate N-terminally labeled peptides, which were selectively eluted by cleavage with TEV protease. Following sequencing by LC-MS/MS, >1300 unique N termini from >650 unique proteins in each sample (Supplementary Dataset 75) were identified.

MetAP substrates within the datasets were identified by the presence of an Abu tag at amino acid 2 within a protein. Biochemical studies of the human MetAPs have demonstrated that they prefer small amino acids in the P1′ position (64), similar to the sequence preference of wild-type stabiligase. Because of this preference, it was predicted that both stabiligase and the stabiligase cocktail would capture MetAP selectivity equally well. Indeed, both stabiligase and the cocktail captured cleavage events at Met-Ala, Met-Gly, Met-Ser, Met-Val, and Met-Thr sequences at similar frequencies (FIG. 5C), accurately reflecting MetAP specificity.

SPP substrates in the datasets were identified by the presence of an Abu tag at a predicted SPP cleavage site as annotated in the Uniprot Knowledgebase (66). In contrast to MetAP, SPP has less P1′ specificity based on a proteome-wide survey of its predicted cleavage sites in Uniprot (n=3,449) (Supplementary Dataset 76), and it was predicted that the stabiligase cocktail would more accurately capture this based on its more efficient labeling of polar and charged sequences. For stabiligase, it was observed that P1′ Asp, Glu, His, and Gln were under-represented compared to the true SPP specificity, while Ala, Gly, Leu, and Ser were over-represented (FIG. SD). The cocktail, in contrast, exhibited lower sequence capture bias, with only P1′ Gln under-represented and P1′ Gly and Ser over-represented. The stabiligase cocktail therefore broadens sequence coverage, enabling capture of neo-N termini that more accurately reflect protease specificity. Although this cocktail for broad sequence coverage was optimized, it is also possible to design custom subtiligase cocktails for the study or proteases of known prime-side specificity using the toolbox of mutants and PILS specificity maps that we have generated.

A Web-Based tool for Subtiligase Variant Selection and PILS Data Analysis.

A web-based tool, ALPINE (α-Amine Ligation Profiling Informing N-terminal modification Enzyme selection) was developed to enable the chemical biology community to leverage the 72 PILS datasets encompassing >25,000 enzyme-substrate pairs that have been collected for selection of optimal subtiligase variants for protein and peptide N-terminal modification applications. ALPINE enables exploration of PILS datasets, identification of the most efficient subtiligase variant for modifying a particular N-terminal sequence, algorithmic selection of customized cocktails for modifying user-defined groups of sequences, and analysis of user-generated PILS datasets. This tool, along with tutorials and sample data, is freely accessible on the web at the website www.wellslab.org/alpine.

FIG. 24 shows that the M222A subtiligase mutant increased the amount of ligation product formed compared to hydrolysis.

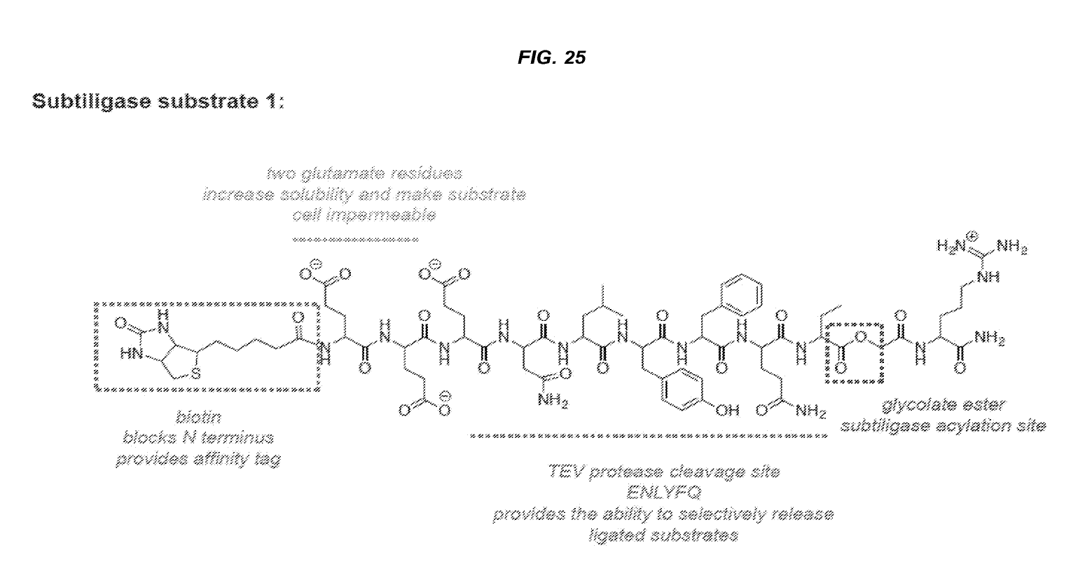

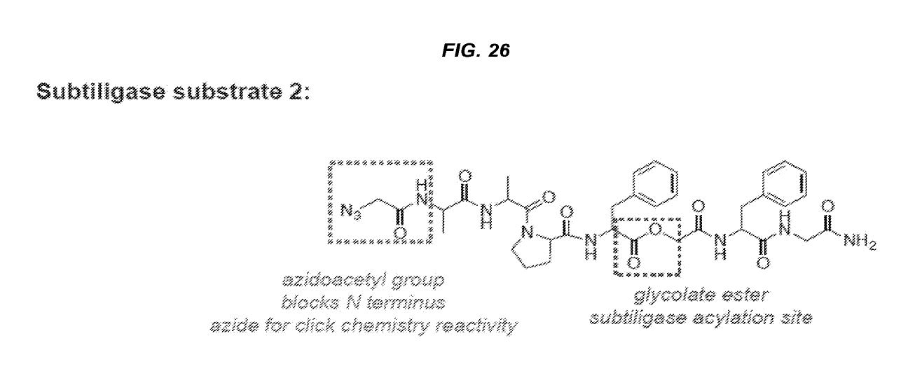

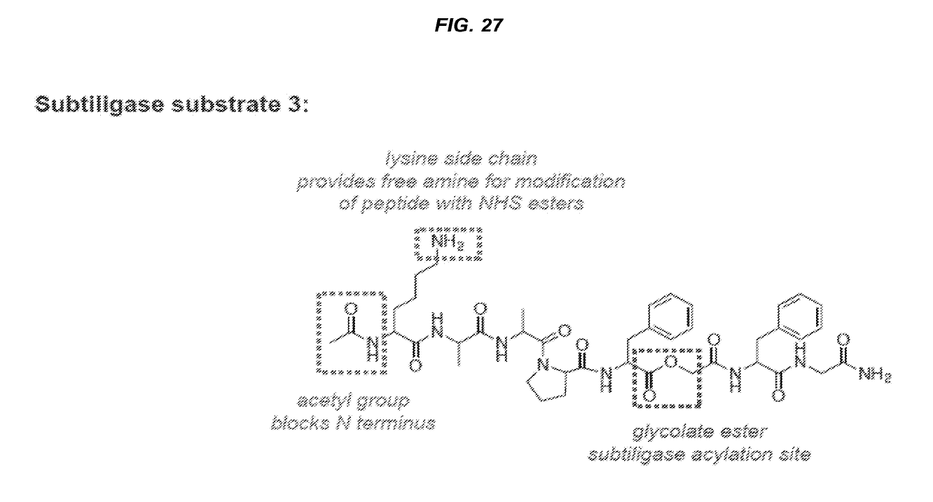

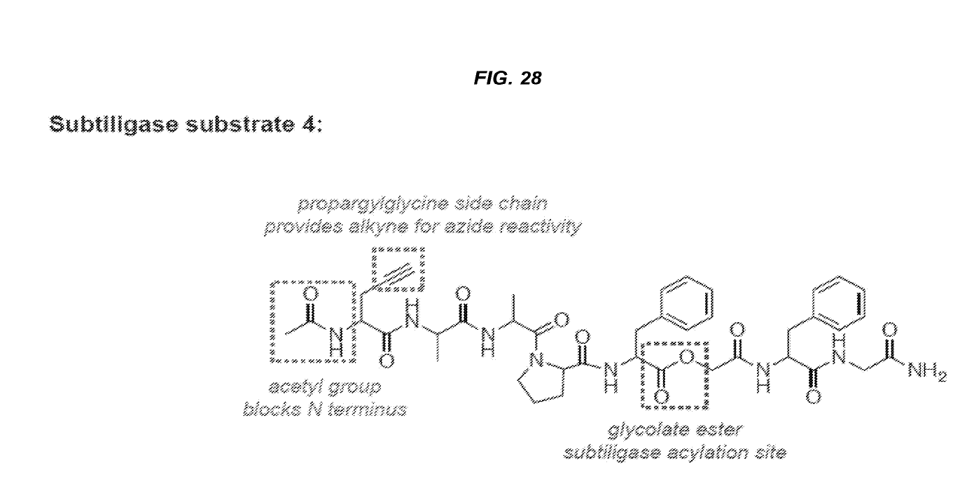

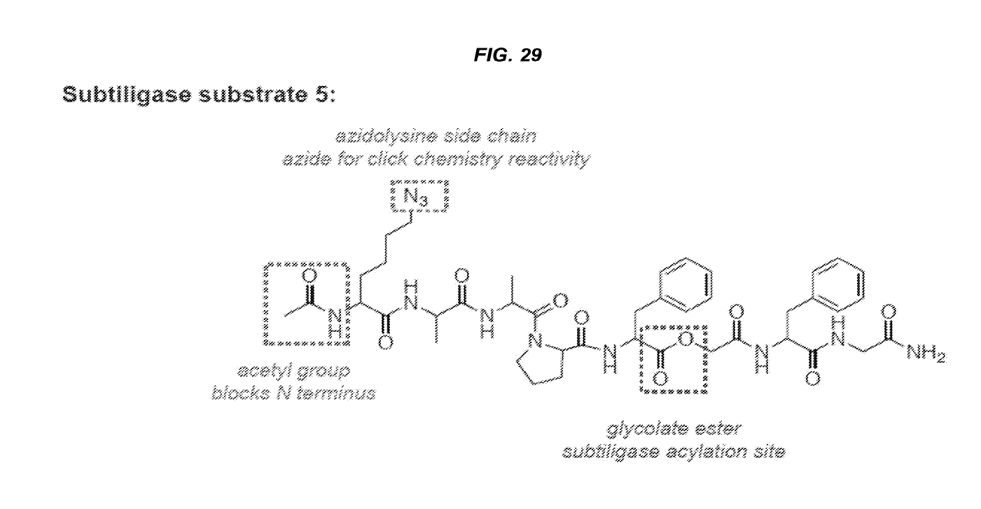

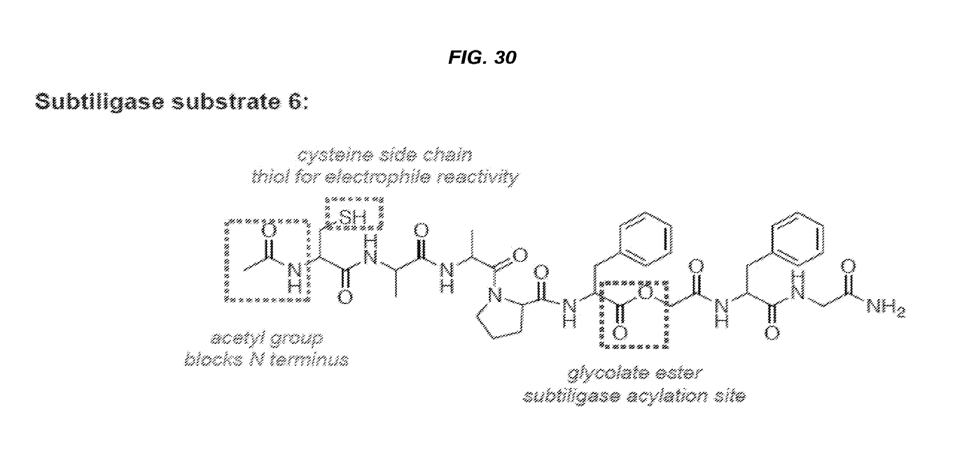

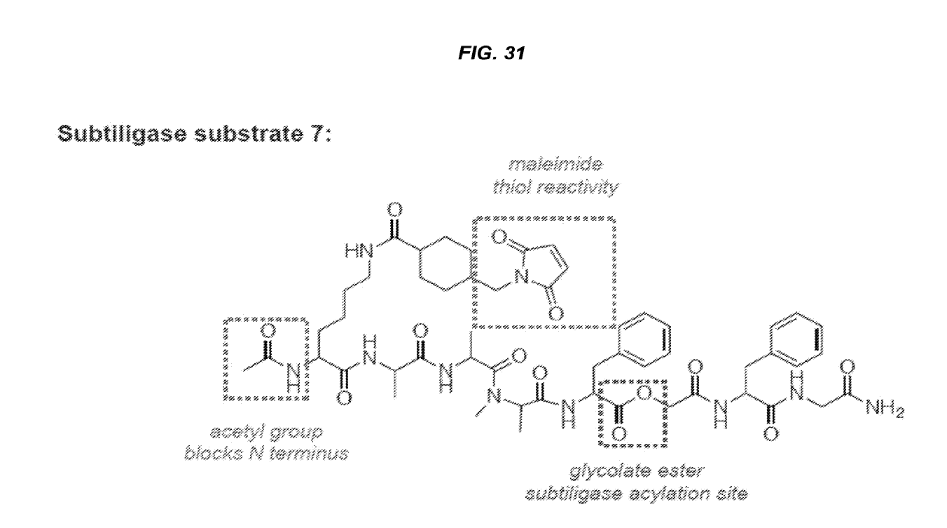

Exemplary embodiments of subtiligase peptide ester substrates can be used with any of the subtiligase variants described herein are provided in FIG. 25-FIG. 31.

### Discussion

Repurposing proteases for peptide ligation has been a goal in the field of protein engineering for the past three decades. In recent years, the pace of discovery and development of peptide ligases has accelerated, opening up new avenues for enzyme-catalyzed site-specific protein bioconjugation. However, the usefulness of these reactions depends on well-defined and predictable sequence specificity. The PILS strategy enables comprehensive characterization of prime-side ligase specificity with a simple natural source of peptide substrates and mass spectrometry-based assay to meet this challenge. PILS can be deployed in combination with mutagenesis to define residues that functionally impact enzyme-substrate specificity and to screen enzyme libraries for protein engineering. Because of the high diversity of the proteome-derived peptide libraries used for PILS, sequence specificity can be analyzed in a context-dependent manner, providing information about subsite cooperativity. PILS is generalizable to other peptide ligase enzymes, as well as to the study of chemical reactions that target the N terminus, providing a platform for the development of new N-terminal modification strategies to enable site-specific protein modification.

Using PILS, the inventors comprehensively characterized prime-side substrate specificity in the designed peptide ligase subtiligase and defined sequences that are refractory to modification by this enzyme. By combining this strategy with alanine-scanning mutagenesis in the area surrounding the active site, ‘hot spot’ residues, Tyr 217 and Phe 189, which determine P1′ and P2′ specificity, respectively were identified. Saturation mutagenesis was performed at these positions to produce a diverse family of subtiligase variants with altered sequence specificity that we characterized with PILS to identify optimal enzyme-substrate pairs. These mutants and their corresponding PILS specificity maps more than double the scope of N-terminal dipeptide sequences that can be targeted for efficient subtiligase modification, from 135 of 400 possible sequences for the wild-type enzyme to 289 of 400 sequences for the new family of peptide ligases. The ALPINE web tool that has been developed (http://www.wellslab.org/alpine) organizes these subtiligase mutant specificity data and enables users to identify mutants optimized for a particular target of interest, thus eliminating the need for genetic engineering to achieve site-specific protein modification in many cases. In cases that do require genetic modification, such as those in which the native protein N terminus is inaccessible or has a sequence that remains resistant to modification, the large number of subtiligase-compatible sequence epitopes enables selection of a sequence that minimizes impact on protein expression level, solubility, and function. It is anticipated that PILS will be applied to re-engineer the specificity of peptide ligases that harbor strict sequence requirements, such as sortase and butelase 1, further augmenting the toolbox of peptide ligases available to protein engineers and chemical biologists.

In combination with the versatile substrates that were developed for one-step and modular protein modification, the mutants that have been engineered will be widely applicable for N-terminal modification with a variety of payloads. Furthermore, subtiligase exhibits broad specificity on the non-prime side and can be used with a wide array of user-designed substrates. Based on its broad sequence compatibility, site selectivity, fast reaction times, mild reaction conditions, and ease of use, it is anticipated that subtiligase-catalyzed protein modification will be widely adopted to advance a variety of scientific fields. Given the ease of site-directed mutagenesis, high expression and facile purification of subtiligase, the task of making fit-to-purpose subtiligases is very practical.

### Methods

### Expression and Purification of Subtiligase and Mutants.

Subtiligase mutants were generated using standard site-directed mutagenesis protocols (1). Subtiligase and variants were expressed as C-terminal His6-tag fusions and secreted from B. subtilis BG2864. Subtiligase and mutants were purified by ethanol precipitation from the culture media followed by Ni-NTA affinity chromatography as described in Supplementary Methods.

### Peptide Synthesis.

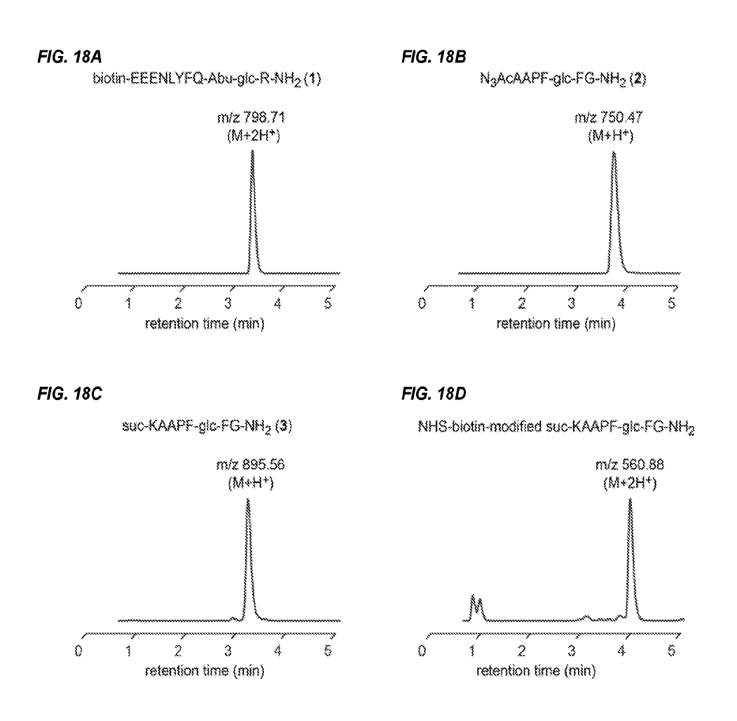

Peptides were synthesized using fluorenylmethyloxycarbonyl (Fmoc) chemistry on Rink Amide AM resin (EMD Millipore) (2). The following side chain protecting groups were used: Arg(Pbf), Gln(Trt), Tyr(tBu), Asn(Trt), Glu(OtBu). Coupling reactions were performed using 5 equiv. of the appropriate Fmoc amino acid, 5 equiv. of diisopropylcarbodiimide (DIC), and 5 equiv. of 1-hydroxy-benzotriazole (HOBt) in N,N-dimethylformamide (DMF) for 1 h at room temperature, except where noted. Fmoc groups were deprotected using a 30 min incubation in 20% (v/v) 4-methylpiperadine in DMF. The glycolic acid moiety was incorporated by coupling the amine of the resin-bound peptide to acetoxyacetic acid (5 equiv.) in the presence of DIC (5 equiv.) and HOBt (5 equiv.) for 1 h, followed by deprotection with 2.5 M hydrazine monohydrate in DMF for 16 h. The amino acid immediately N-terminal to the glycolic acid group (5 equiv.) was coupled to the peptide in the presence of 1 M DIC and 1 mol % N,N-dimethylaminopyridine for 1 h (3). Biotin (5 equiv.) was dissolved in dimethylsulfoxide (DMSO) and coupled to the peptide using 5 equiv. DIC and 5 equiv. HOBt. Peptides were cleaved and side chains were deprotected by incubating the resin with 95:2.5:2.5 ratio of trifluoroacetic acid (TFA), water, and triisopropylsilane (TIPS). The solution was concentrated to 5 mL on a rotary evaporator and the peptide was precipitated by addition of 9 volumes of diethyl ether and washed twice with diethyl ether. Peptides were purified by C18 reverse-phase HPLC using a gradient from 0.1% TFA in water to 0.1% TFA in acetonitrile. Acetonitrile was removed using a vacuum centrifuge and peptides were lyophilized. Lyophilized peptides were dissolved in DMSO and stored at -80° C. until use. LC-MS characterization data for each peptide is shown in FIG. 18A-FIG. 18D.

### Preparation of Proteome-Derived Peptide Libraries.

2×YT (50 mL) was inoculated with a single colony of E. coli XL10 and incubated overnight at 37° C. with shaking at 200 rpm. Cells were harvested by centrifugation at 4,000×g for 15 min at 4° C. and resuspended in 50 mL of lysis buffer (10 mM HEPES, pH 7.5, 1 mM PMSF, 10 mM EDTA). Cells were lysed by three passes through a microfluidizer at 15,000 psi. Insoluble material was removed by centrifugation at 10,000×g for 20 min at 4° C. DNA was precipitated by dropwise addition of 10% (w/v) streptomycin sulfate to a final concentration of 1% (w/v) and removed by centrifugation at 10,000×g for 20 min at 4° C. Protein concentration was determined by BCA assay and subsequent steps were carried out on a total of 10 mg of protein at 2 mg/mL. The lysate was adjusted to 100 mM HEPES, pH 7.5 and DTT (1 M) was added to 5 mM. Following a 1 h incubation at room temperature, iodoacetamide (500 mM) was added to 10 mM and the sample was incubated in the dark for 1 h at 37° C. Protein was precipitated by addition of 15% (w/v) trichloroacetic acid (TCA) followed by an overnight incubation at −20° C. The sample was centrifuged at 20,000×g for 10 min and the pellet was washed twice with ice-cold methanol. The pellet was solubilized by ultrasonication in 5 mL 20 mM NaOH (20% amplitude, 5 s on/1 s off) and adjusted to 200 mM HEPES, pH 7.5. Insoluble material was removed by centrifugation at 20,000×g for 20 min at 4° C. The protein concentration of the supernatant was determined by BCA assay and protein was digested overnight at 37° C. with a 1:100 (w/w) ratio of mass-spectrometry grade trypsin or Glu-C. After digestion, 1 mM PMSF (for trypsin) or 1 mM PMSF and 0.5 mM diisopropylfluorophosphate (for Glu-C) was added to inhibit the digest protease. Reduction and alkylation were repeated and peptide libraries were purified by C18 solid-phase extraction and eluted in 80% acetonitrile/20% water. Libraries were concentrated in a vacuum centrifuge and diluted three times with water to remove acetonitrile, diluted to 2 mg/mL in water, and stored at -80° C. until further use.

### Subtiligase Specificity Profiling Using Proteomic Identification of Ligation Sites (PILS).

Specificity profiling reactions were initiated by addition of subtiligase or variant (1 μM) to a reaction mixture containing peptide library (1 mM, concentration estimated based on an average protein molecular of 30 kDa, see Supplementary Methods for details), biotinylated peptide ester 1 (0.2 mM), and 100 mM tricine, pH 8.0. After 1 h, reactions were quenched by addition of 1 volume of 8 M guanidine hydrochloride. Biotinylated peptides were enriched on High-Capacity Neutravidin resin (Thermo Fisher Scientific) (0.25 mL of 50% resin slurry). The resin was washed five times with 0.5 mL 4 M guanidine hydrochloride and five times with TEV elution buffer (100 mM ammonium bicarbonate, 2 mM DTT). The resin was resuspended in 0.25 mL TEV elution buffer and incubated with TEV protease (10 μg) for 2 h to selectively elute biotinylated peptides. Resin was removed from the eluted peptides using a spin filter. The solution containing the eluted peptides was adjusted to 5% TFA, incubated at room temperature for 10 min, and spun at 20,000×g to remove precipitated TEV protease. Peptides were then desalted on C18 OMIX tips, dried, dissolved in 10 μL 0.1% formic acid, and analyzed by LC-MS/MS.

### LC-MS/MS Data Collection.

LC-MS/MS analysis was performed on an Acclaim PepMap RSLC column (75 μm×15 cm, 2 μm particle size, 100 Å pore size, Thermo Scientific) using a Thermo Dionex UltiMate 3000 RSLCnano liquid chromatography system coupled to a Thermo Q-Exactive Plus hybrid quadrupole-Orbitrap mass spectrometer. Mobile phase A was 0.1% formic acid and mobile phase B was 0.1% formic acid, 80% acetonitrile. Samples (5 μL) were loaded over 15 min at 0.5 μL/min in mobile phase A and peptides were eluted at 0.3 μL/min with a linear gradient from mobile phase A to 40% mobile phase B over either 30 min (for PILS experiments) or 125 min (for N terminomics experiments). Data-dependent acquisition of MS data was performed using Thermo Xcalibur software scanning a mass range from 300-1,500 m/z.

### Mass Spectrometry Data Analysis.

Peak lists from Thermo RAW files were generated using MSConvert (Proteowizard). Peptides were identified from the E. coli or human SwissProt database using Protein Prospector (UCSF) with a false discovery rate of <1%. The parent ion tolerance was set at 6 ppm and the fragment ion tolerance was set at 20 ppm and two missed cleavages were allowed. Search parameters included carbamidomethylation at Cys as a constant modification and aminobutyric acid (Abu) at peptide N termini, acetylation at protein N termini, oxidation at Met, pyroglutamate formation at N-terminal Gln, and Met excision at protein N termini as variable modifications. Trypsin specificity was defined to include cleavage C-terminal to Arg or Lys, and Glu-C specificity was defined to include cleavage C-terminal to Glu or Asp. For analysis of PILS data, the appropriate specificity was required at both the N- and C-terminal ends of the peptide. For analysis of N terminomics datasets, the appropriate specificity was required at only the C terminus of the peptide to enable identification of protease cleavage events of different specificity. For reference trypsin and Glu-C datasets used in PILS analysis, data were analyzed similarly, omitting the Abu variable modification.

### PILS Specificity Data Analysis.

PILS analysis was implemented using custom Python scripts that are included in the Supplementary Materials. Lists of identified peptides were filtered for bona fide subtiligase substrates based on the presence of an Abu modification at the peptide N terminus. Peptides from trypsin and Glu-C datasets were combined and the frequency with which each amino acid appeared in each position was compared to the frequency in the combined trypsin and Glu-C reference sets. An enrichment score (z) was calculated according to the following formula:

$$z = \frac{X - μ}{σ}$$

where X is the frequency of the amino acid in the enriched, Abu-tagged sample, μ is the frequency of the amino acid in the reference sample, and σ is the standard deviation. A positive enrichment score indicates that an amino acid is enriched compared to the input libraries, while a negative enrichment score indicates that an amino acid is de-enriched compared to the input libraries. For analysis of dipeptide sequences, the same approach was used, except the frequency of the dipeptide sequence at the N termini of peptides in the sample and reference sets was compared. The tryptic reference set contained 5,720 peptides and the Glu-C reference set contained 4,278 peptides (Supplementary Dataset 77). Individual enriched datasets generally contained 1,000-4,000 peptides (Supplementary Datasets 1-73). Hierarchical clustering of enrichment scores was performed using the ‘heatmap’ function in R (www.r-project.org).

### Kinetic Analysis of Subtiligase Mutants.

Kinetic analysis of subtiligase mutants was performed using a FRET-based assay for peptide ligation as described in the Supplementary Methods below.

N Terminomics Analysis in E. coli and Jurkat Cell Lysate.

E. coli XL10 were lysed by three passes through a microfluidizer at 15,000 psi in 100 mM tricine, pH 8, 150 mM NaCl, 100 μM PMSF, 100 μM AEBSF, 2.5 mM EDTA. Insoluble material was removed by centrifugation at 20,000×g for 20 min at 4° C. Biotinylated subtiligase substrate 1 was added to the supernatant at a final concentration of 2.5 mM. The reaction was initiated by addition of the appropriate subtiligase variant (1 μM) and allowed to proceed for 1 h at room temperature on an end-over-end mixer. After labeling, biotinylated N-terminal peptides were enriched as described previously (4-6) and analyzed by LC-MS/MS. All experiments were performed in duplicate.

For N terminomics studies of Jurkat lysate, cells were lysed by ultrasonication (20% amplitude, 5 s/1 s on/off) in 400 mM tricine, pH 8, 4% (w/v) SDS, 100 μM PMSF, 100 μM AEBSF, 2.5 mM EDTA. Insoluble material was removed by centrifugation at 20,000 x g for 20 min at room temperature. The sample was reduced by boiling for 15 min in the presence of 5 mM TCEP and alkylated by 1 h incubation at room temperature in the presence of 10 mM iodoacetamide. DTT (25 mM) was added to quench the remaining iodoacetamide and Triton X-100 was added to a final concentration of 2.5% (v/v). The sample was diluted four-fold with water and biotinylated subtiligase substrate 1 was added to a final concentration of 2.5 mM. The reaction was initiated by addition of stabiligase or the stabiligase cocktail (4 μM) and allowed to proceed at room temperature for 1 h. After labeling, biotinylated N-terminal peptides were enriched as described previously (4, 5, 7).

### Purification of GFP Variants for Protein Bioconjugation.

GFP and N-terminal variants were expressed as His₆-SUMO tag fusion proteins and purified using Ni-NTA affinity chromatography. After affinity purification, the His₆-SUMO tag was cleaved using Senpl protease as previously described (8) and the cleaved His₆-SUMO was removed using Ni-NTA affinity chromatography.

### Purification of Recombinant Antibodies.

Recombinant antibodies were expressed in E. coli from a single vector with the light chain fused to a PelB leader sequence and the heavy chain fused to an STII signal sequence for secretion to the periplasm (9). Cells were lysed with B-PER (ThermoFisher Scientific) and the lysate was heated to 60° C. for 20 min. After removal of insoluble material by centrifugation, antibodies were purified on a HiTrap Protein A sepharose column and exchanged into PBS for storage.

### Protein Bioconjugation Reactions.

Purified protein was diluted to 50 μM in 100 mM tricine, pH 8.0 containing 5 mM of the subtiligase substrate to be conjugated. The reaction was initiated by addition of subtiligase or the appropriate variant (1 μM) and allowed to proceed for 1 h at room temperature. Protein was then exchanged into PBS using a 0.5 mL Zeba desalting spin column (ThermoFisher Scientific) and the completeness of the reaction was analyzed on a Xevo G2-XS mass spectrometer equipped with a LockSpray (ESI) source and Acquity Protein BEH C4 column (2.1 mm inner diameter, 50 mm length, 300 Å pore size, 1.7 μm particle size) connected to an Acquity I-class liquid chromatography system (Waters). Deconvolution of mass spectra was performed using the maximum entropy (MaxEnt) algorithm in MassLynx 4.1 (Waters).

Modification of Peptide 3 with Biotin.

Peptide 3 (100 mM in DMF, 20 μL) was mixed with EZ-Link NHS-biotin (110 mM in DMF, 20 μL) and incubated for 1 h at room temperature. Excess NHS-biotin was quenched by addition of 20 μL of water followed by an overnight incubation at room temperature. The reaction mixture was used without further purification in protein bioconjugation reactions.

Modification of Peptide 2-Modified Proteins with DBCO Reagents.

Following protein bioconjugation with peptide 2, proteins were desalted into PBS three times using 0.5 mL Zeba desalting spin columns (ThermoFisher Scientific). Proteins (50 μM) were then modified with the appropriate DBCO reagent (130 μM) by incubating for 2-16 h at room temperature. Excess DBCO reagent was removed by exchanging into PBS using a 0.5 mL Zeba desalting spin column.

### GFP-αGFP Co-Localization Experiments.

HEK293T cells modified with a doxycycline-inducible cell surface GFP expression system were plated at 10,000 cells per well in a 96-well flat-bottom tissue culture plate. GFP expression was induced at 50% confluency by addition of 1 μg/mL doxycycline to the culture medium, or an equal volume of water as a negative control. After 18 h, cells were washed three time with PBS containing 3% BSA and stained with 0.1 μg/mL αGFP-rAb in PBS +3% BSA for 30 min at room temperature. Cells were washed three times with PBS +3% BSA and imaged using a Zeiss AxioObserver Z1 inverted fluorescence microscope. Experiments were performed in triplicate.

### Computer Code Availability.

Python and R scripts used for data analysis are included herein. Additionally, the ALPINE web application (www.wellslab.org/alpine) includes web interfaces for many of these scripts.

### Data Availability.

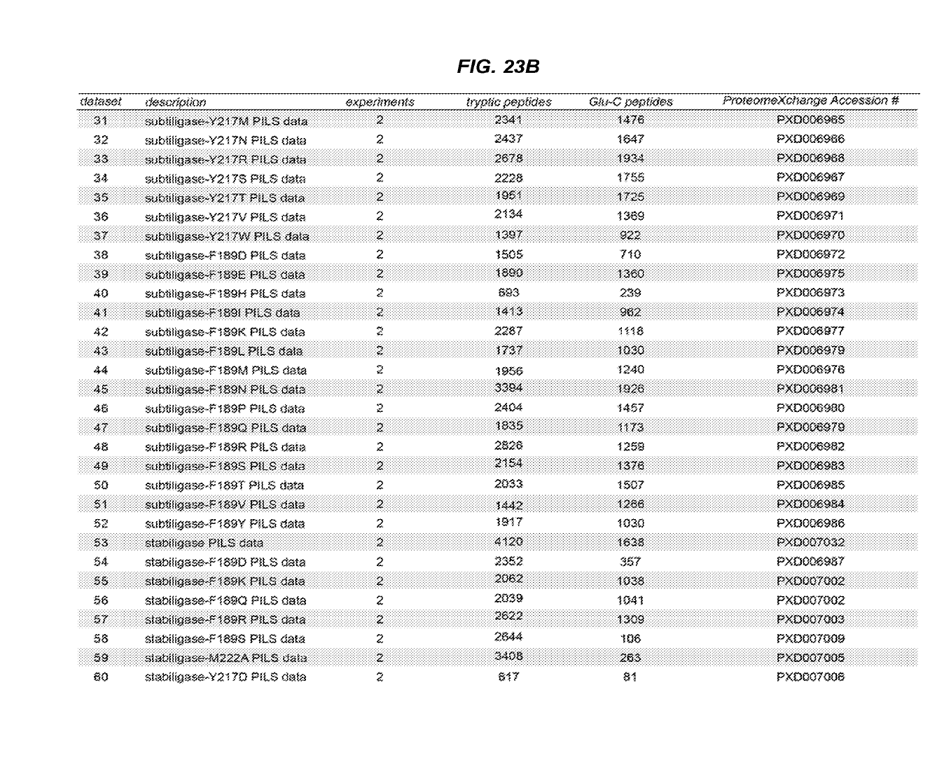

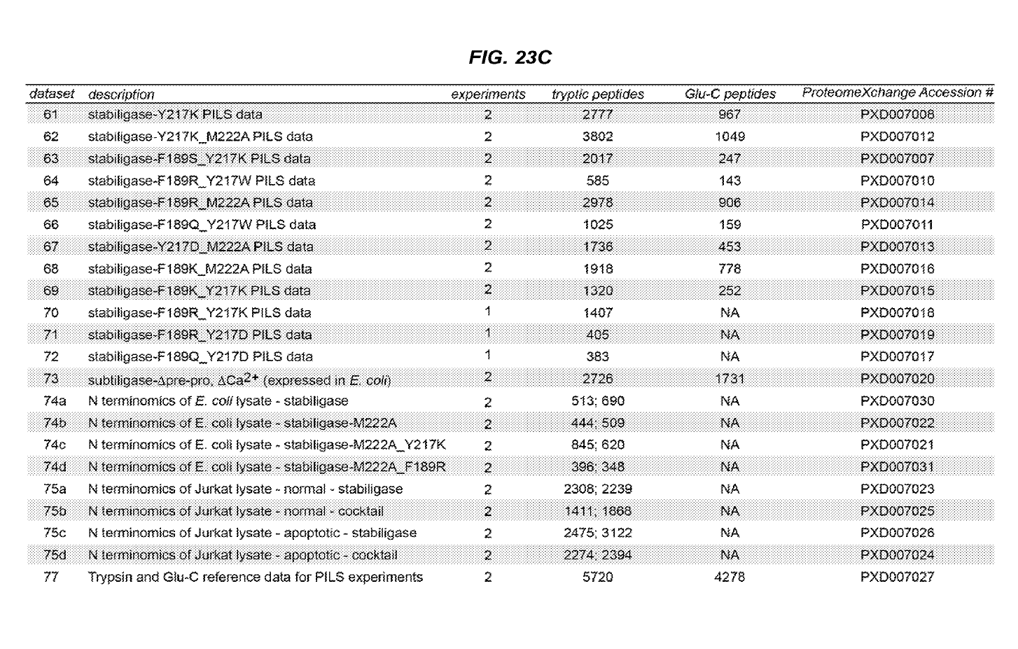

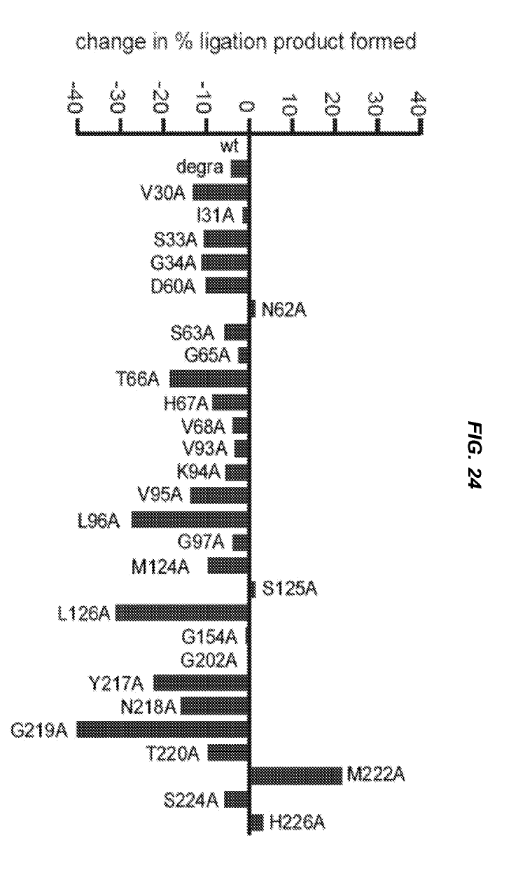

All data generated or analyzed for this study are available within the paper and its associated supplementary information files, or from the corresponding author upon reasonable request. Additionally, raw mass spectrometry data and search results have been deposited in the ProteomeXchange repository under the accession numbers listed in FIG. 23A, FIG. 23B, and FIG. 23C.

### Supplementary Methods

### Construction of Plasmids.

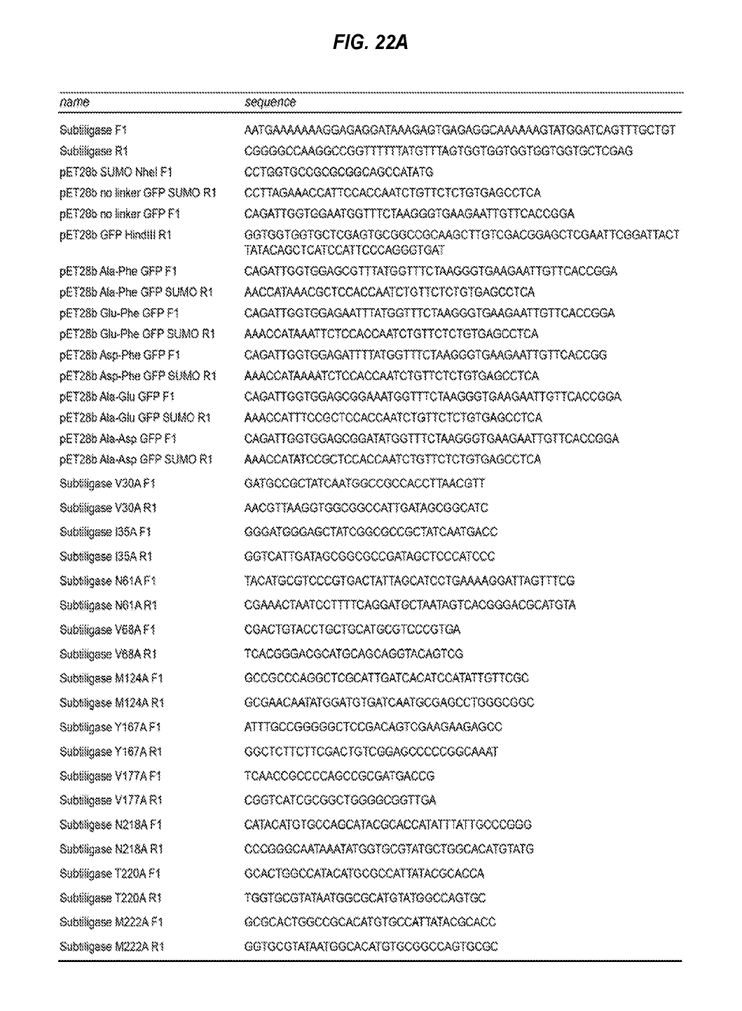

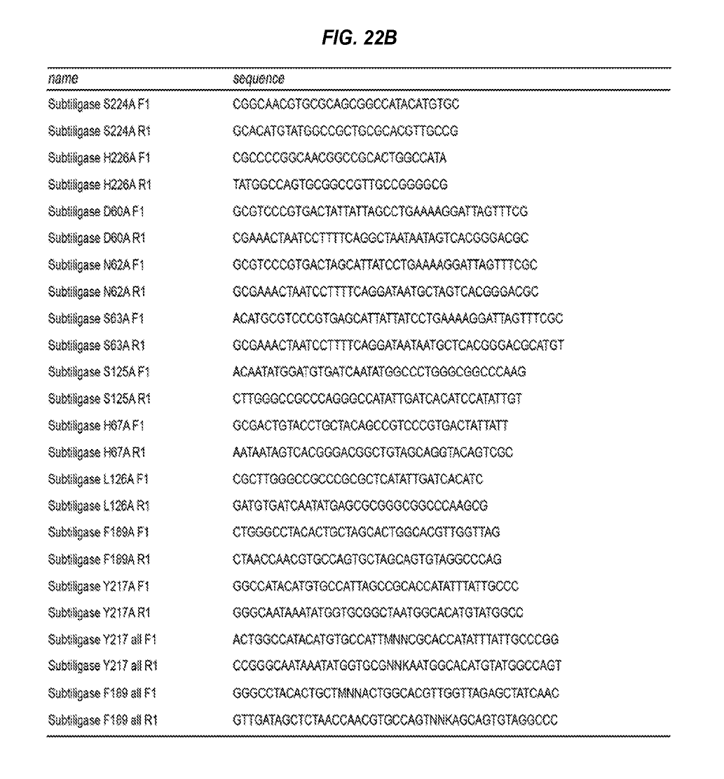

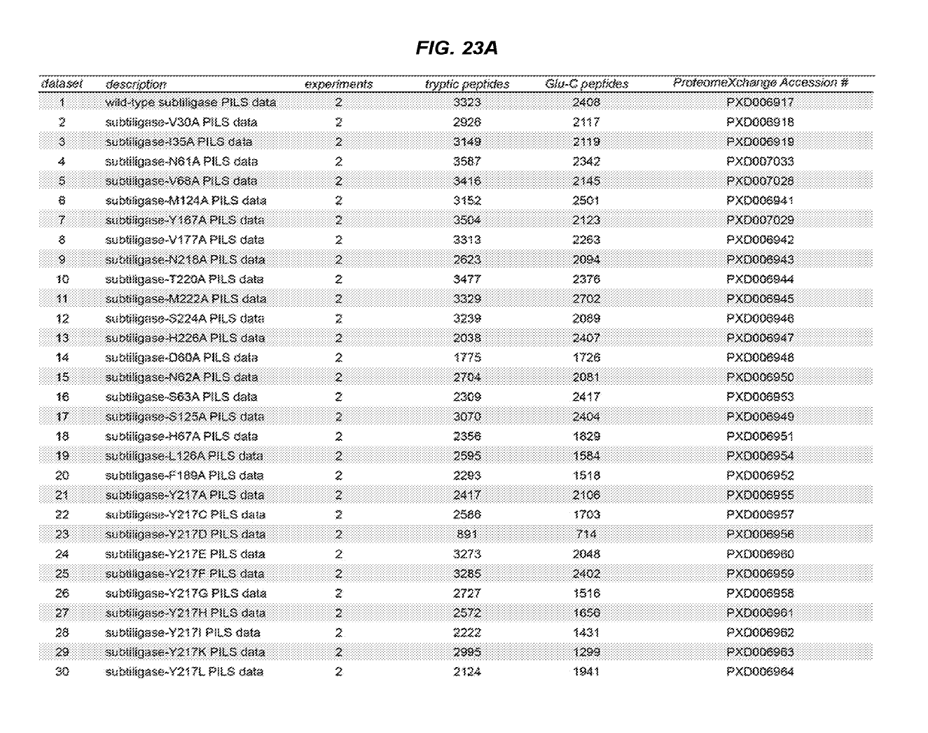

Plasmids were constructed using standard Gibson cloning methods with E. coli XL10 as the cloning host. PCR amplifications were performed using KOD Hot Start Polymerase (EMD Millipore) using the oligonucleotides listed in FIG. 22A and FIG. 22B. All plasmids were verified by Sanger sequencing (Quintara Biosciences).

### Pro-Subtiligase-His₆.

A codon-optimized synthetic gene encoding pre-pro-subtiligase-His₆ was purchased from Integrated DNA Technologies. The DNA sequence are mature protein sequence are given below. The synthetic gene was PCR amplified using primers Subtiligase F1 and Subtiligase R1 (FIG. 22A and FIG. 22B) and inserted between the EcoRI and BamHI sites of pBS42 (1) using Gibson assembly.

DNA sequence of synthetic gene (SEQ ID NO:84):

GTGAGAGGCAAAAAAGTATGGATCAGTTTGCTGTTTGCTTTAGCGTTAA
TCTTTACGATGGCGTTCGGCAGCACATCCTCTGCCCAGGCGGCCGGTAA
ATCCAACGGTGAGAAAAAATATATTGTAGGCTTCAAACAAACCATGAGC
ACCATGTCGGCTGCCAAAAAAAAAGACGTCATTTCAGAGAAGGGTGGGA
AGGTACAAAAACAGTTCAAATATGTAGATGCGGCCTCCGCCACGTTGAA
CGAAAAGGCGGTAAAAGAACTGAAAAAAGATCCGTCAGTGGCATACGTA
GAAGAAGATCATGTCGCGCATGCTTATGCTCAAAGCGTCCCGTACGGCG
TCTCACAGATCAAGGCACCGGCGCTGCACAGCCAGGGTTATACCGGCTC
CAACGTTAAGGTGGCGGTCATTGATAGCGGCATCGATAGCTCCCATCCC
GACCTCAAAGTTGCCGGCGGCGCTTCTATGGTGCCAAGCGAAACTAATC
CTTTTCAGGATAATAATAGTCACGGGACGCATGTAGCAGGTACAGTCGC
CGCTTTGAATAATTCTATCGGCGTGCTGGGTGTTGCGCCGAGCGCGTCA
CTCTACGCCGTGAAAGTGCTGGGCGCGGACGGCAGCGGACAATATAGTT
GGATTATTAATGGCATCGAGTGGGCCATCGCGAACAATATGGATGTGAT
CAATATGAGCCTGGGCGGCCCAAGCGGCAGTGCTGCCTTAAAAGCGGCG
GTGGATAAAGCTGTGGCAAGTGGGGTCGTCGTGGTGGCAGCGGCGGGCA
ATGAAGGCACGAGTGGCTCTTCTTCGACTGTCGGATACCCCGGCAAATA
CCCGTCGGTCATCGCGGTTGGGGCGGTTGATAGCTCTAACCAACGTGCC
AGTTTTAGCAGTGTAGGCCCAGAATTAGATGTGATGGCGCCAGGTGTGT
CTATCCAGAGCACACTCCCGGGCAATAAATATGGTGCGTATAATGGCAC
ATGTATGGCCAGTGCGCACGTTGCCGGGGCGGCGGCCCTGATCTTAAGT
AAACATCCAAACTGGACCAACACCCAGGTGCGTAGCAGTTTGGAAAACA
CCACCACGAAACTGGGTGATTCTTTTTATTACGGGAAAGGTCTCATCAA
TGTTCAAGCGGCCGCCCAACTCGAGCACCACCACCACCACCACTAA

Protein sequence of mature subtiligase (SEQ ID NO:85)::

AQSVPYGVSQIKAPALHSQGYTGSNVKVAVIDSGIDSSHPDLKVAGGAS
FVPSETNPFQDNNSHGTHVAGTVAALDNSIGVLGVAPSASLYAVKVLGA
DGSGQYSWIISGIEWAIANNMDVINLALGGPSGSAALKAAVDKAVASGV
VVVAAAGNEGTSGSSSTVGYPGKYPSVIAVGAVDSSNQRASFSSVGPEL
DVMAPGVSIQSTLPGNRYGAYSGTCMASAHVAGAAALILSKHPNWTNTQ
VRSSLENTTTKLGDSFYYGKGLINVQAAAQLEHHHHHH

Plasmids encoding subtiligase variants were constructed using oligonucleotide primers encoding the desired mutation (FIG. 22A and FIG. 22B). Site-directed mutagenesis reactions contained forward and reverse primers (0.5 μMeach), pBS42-Subtiligase-His₆ template (100 ng), dNTPs (0.2 mM each), MgSO₄ (2.5 mM), KOD Hot Start DNA polymerase buffer (1×), and KOD Hot Start DNA polymerase (0.02 U/μL). The reaction mixture was subjected to the following thermocycling conditions: 95° C. for 2 min; 16 cycles of 95° C. for 20 s, 55° C. for 10 s, 72° C. for 3 min 30 s; a final extension at 72° C. for 7 min. Reaction mixtures were digested with Dpnl (0.8 U/μL) for 1 h at 37° C. and transformed into E. coli XL10.

pET28b-His6-Smt3-eGFP and Variants. S. Cerevisiae.

Smt3 was amplified from pET28b-Smt3 (a gift from L. Pack) using primers pET28b SUMO NheI F1 and pET28b no linker GFP SUMO R1 and eGFP was amplified from pBH4-eGFP (a gift from S. Coyle) using primer pET28b no linker GFP F1 and pET28b GFP HindIII R1. Both PCR products were inserted between the NheI and HindIII sites of pET28b using Gibson assembly. To construct vectors for expression of eGFP variants with N-terminal dipeptide extensions, Smt3 was amplified with the universal primer pET28b SUMO NheI F1 and the appropriate reverse primer listed in FIG. 22A, and by amplifying eGFP with the appropriate forward primer listed in FIG. 22A and the universal primer pET28b GFP HindIII R1. PCR products were then inserted between the NheI and HindIII sites of pET28b.

### Expression and Purification of Subtiligase-His₆ and Variants.

E. coli ER1821 were transformed with each subtiligase expression plasmid and concatameric DNA was prepared using a QlAprep Spin Miniprep Kit (Qiagen), omitting the Buffer PB wash. B. subtilis BG2864 were transformed with the concatameric DNA and grown on LB agar supplemented with 5 μg/mL chloramphenicol. 2×YT (5 mL) containing 12.5 μg/mL chloramphenicol was then inoculated with a single colony and grown overnight at 37° C. with shaking at 200 rpm. 2×YT (50 mL) supplemented with 12.5 μg/mL chloramphenicol and 5 mM CaCl₂ was inoculated with the saturated overnight culture to an OD₆₀₀ of 0.03 and grown in a baffled flask at 37° C. with shaking at 200 rpm for 20-24 h. Cells were then removed by centrifugation at 4,000×g for 15 min at 4° C. Secreted subtiligase was precipitated out of the media by addition of 3 volumes of cold ethanol and pelleted by centrifugation at 4,000×g for 15 min at 4° C. Pellets were resuspended in 10 mL Ni-NTA wash buffer (50 mM sodium phosphate, pH 8.0, 300 mM NaCl, 20 mM imidazole) and insoluble material was removed by centrifugation at 4,000×g for 15 min at 4° C. The supernatant was allowed to bind to HisPur Ni-NTA resin from a HisPur Ni-NTA spin column for 1 h at 4° C. The resin was collected by centrifugation at 500×g for 5 min, resuspended in 400 μL Ni-NTA wash buffer, and loaded into the HisPur spin column. The column was washed with 4×400 μL Ni-NTA wash buffer by centrifugation at 700×g for 2 min. Subtiligase variants were eluted with 3×400 μL Ni-NTA elution buffer (50 mM sodium phosphate, pH 8, 300 mM NaCl, 250 mM imidazole) and quantified by absorbance at 280 nm. HisPur spin columns were discarded after purification and a new spin column was used to purify each mutant to avoid the possibility of cross-contamination. The purified protein was buffer exchanged into 100 mM tricine, pH 8, 5 mM DTT, 10% glycerol by five cycles of 10-fold concentration and dilution in an Amicon Ultra centrifugal filter unit (0.5 mL, 3,000 MWCO). Single-use aliquots were flash frozen and stored at −80° C. Protein molecular weights were verified by LC-MS (FIG. 20A and FIG. 20B).

### Estimation of Peptide Library Molarity.

Libraries were stored as 2 mg/mL stock solutions by determining the concentration with a bicinchoninic acid (BCA) assay. The molarity of trypsin and Glu-C peptide libraries was estimated by taking into account the average length of an E. coli protein (300 amino acids (a. a.)) (2), the average length of a tryptic peptide (10 a. a.) (3), and the average molecular weight (MW) of an amino acid (110 Da).

Calculations are shown below.

$$Average protein MW = 300 a . a . \times \frac{110 Da}{a . a .} = 33 \text{,} 000 Da \\ Average number of peptides per protein = 300 a . a . \times \frac{1 peptide}{10 a . a .} = 30 peptides$$

$$Peptide library molarity = \frac{2 mg}{mL} \times \frac{1 mmol protein}{33 \text{,} 000 mg} \times \frac{30 peptide}{protein} \times \frac{1000 mL}{L} = 1.8 mM pepide$$

### Kinetic Analysis of Subtiligase Mutants.

The peptide ligation activity of subtiligase was measured using the FRET-based assay shown schematically in FIG. 9A. Assays were performed in 96-well plates in 200 μL total volume containing 100 mM tricine, pH 8.0, 5 mM DTT, 20 μM Pacific Blue-GAAPF-glc-RK(Dabcyl) (SEQ ID NO:86; a subtiligase ester substrate) and 0, 6.25, 12.5, 25, 50, 100, or 200 μM AFAK(FAM) (SEQ ID NO:87). Reactions were initiated by the addition of subtiligase or subtiligase variant to a final concentration of 25 nM. Fluorescence was monitored over time in a Molecular Devices SpectraMax M5 plate reader with excitation at 405 nm and emission at 450 nm (hydrolysis product) and 520 nm (ligation product). A standard curve of Pacific Blue-GAAPFAFAK(FAM) (SEQ ID NO:88) was constructed to correlate fluorescence intensity with ligation product concentration. A plot of AFAK(FAM) (SEQ ID NO:87) concentration vs. observed rate was fit to a line to determine the relative k_{cat}/K_{M} for each mutant enzyme.

### Measurement of rAb Affinities.

The affinities of αGFP rAbs for GFP were measured using biolayer interferometry on an Octet RED 384 system (ForteBio). The modified or unmodified αGFP rAbs were diluted to 300 nM in PBS containing 0.05% Tween 20, 0.2% BSA, and 10 μM biotin. The rAbs were immobilized on 2^{nd} Generation Dip and Read Anti-Human-Fab-CH1 sensor tips (ForteBio). Binding of GFP to the immobilized rAbs was assessed by loading serial dilutions of recombinant GFP onto the sensors. Results were fit using the Data Analysis 9.0 software provided with the Octet RED 384 to determine dissociation constants (K_{D}s).

Cell Culture for N terminomics Studies.

Cell lines used in this study were tested annually for mycoplasma contamination. Jurkat E6.1 (a gift from Kole Roybal, Lim lab, UCSF) cells were grown in RPMI-1640 media supplemented with 10% fetal bovine serum, 2 mM L-glutamine, and 1% penicillin-streptomycin to a density of 1×10⁶ cells per mL. The day before harvest, cells were split by two-fold and treated with either 50 μM etoposide for 12 h or an equal volume of DMSO. Cell death was assessed using the CellTiterGlo assay (Promega) according to the manufacturer's instructions. Cells were harvested at 300×g for 5 min, washed twice with PBS, and stored at −80° C. until use.

Analysis of Signal Peptide Peptidase N terminomics Experiments.

A cocktail of three stabiligase enzymes was selected for maximum coverage of the 400-dipeptide sequence space, omitting sequences with P1′ or P2′ Arg or Lys, using a custom Python script (Supplementary Script 11) that implements a greedy set-cover algorithm (4). A pseudocode description of this algorithm is as follows:

1: repeat

2: pick the mutant that covers the maximum number of uncovered dipeptide sequences with an enrichment score >0

3: mark the sequences labeled by the chosen mutant as covered

4: until done

For two replicate experiments with either stabiligase or the algorithmically selected stabiligase cocktail, peptides with Abu tags at an annotated signal peptide cleavage set were extracted from each dataset using a custom Python script (Supplementary Script 13). For comparison, the amino acid composition of the P1′ position of all annotated human signal peptide peptidase substrates in Uniprot was determined by identifying signal peptide-containing proteins and performing in silico signal peptide peptidase cleavage on them using a custom Python script (Supplementary Script 12). The P1′ amino acid frequencies for each experimental dataset (n=50-69) were compared with the frequencies from the in silico digest (n=3,449) and the statistical significance of the frequency difference was assessed using an unpaired t-test with a p-value cutoff of 0.05 in GraphPad Prism.

### EXAMPLE 2

### Engineering Peptide Ligase with Altered Specificity by Proteomic Identification of Ligation Sites

We set out to develop a platform for rapid and quantitative characterization and engineering of peptide ligase specificity, and to deploy this platform to expand the toolbox of enzymes available for site-specific modification of protein and peptide N termini. We sought to: (a) identify subtiligase variants capable of modifying specific N-terminal sequences that were previously recalcitrant toward modification; (b) identify cocktails of subtiligase variants that maximally expand the sequence space that can be modified by subtiligase; (c) apply these subtiligase variants for site-specific protein conjugation; and (d) apply subtiligase cocktails to achieve more comprehensive sequencing of the cellular N terminome.

We developed a strategy for comprehensive characterization of peptide ligase specificity that utilizes database-searchable, proteome-derived peptide libraries as ligase substrates. This method termed Proteomic Identification of Ligation Sites (PILS) enables selective isolation of ligated peptides and sequencing by liquid chromatography-tandem mass spectrometry (LC-MS/MS) for rapid determination of positional enrichment or de-enrichment of each amino acid at each P′ site (FIG. 1B).

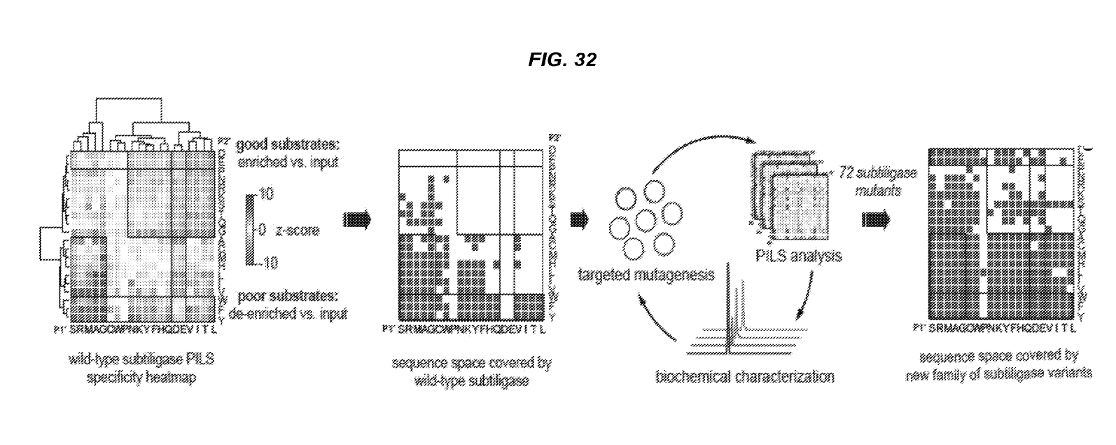

We used this method to comprehensively characterize wild-type subtiligase specificity, and deployed it as an engineering platform to identify mutants with altered specificity (FIG. 32).

We applied PILS to comprehensively characterize subtiligase specificity, and combined it with alanine scanning mutagenesis at 20 sites to systematically identify sites that affect substrate sequence selectivity. We then deployed PILS to screen single-site saturation mutagenesis libraries at the most impactful sites, enabling identification of a panel of variants that redirect subtiligase activity toward sequences that were previously refractory to modification. The subtiligase variants can be applied to site-specific protein conjugation.

A variety of proteins can be modified with subtiligase-catalyzed bioconjugation. The subtiligase variants that we identified in our PILS-based screen expand the scope of protein substrates that can be modified in high yield. We developed a modular bioconjugation protocol by using subtiligase to incorporate a bioorthogonal azide group at the protein N terminus. This azide can be modified after incorporation into the protein by copper-free azide-alkyne click chemistry with commercially available dibenzyocyclooctynes (DBCOs). We incorporated an azide into αGFP to produce biotinylated αGFP (DBCO-biotin), fluorescent αGFP (DBCO-Cy3), an αGFP-drug conjugate (DBCO-MMAE), an oligonucleotide-modified αGFP (5′-DBCO-oligonucleotide), and a PEGylated αGFP (DBCO-PEG 5000) (FIG. 3D and FIG. 4A-FIG. 4F). Importantly, these modifications led to only small decreases in affinity of the αGFP rAb for GFP, demonstrating that protein function is maintained upon modification (FIG. 4F).

In conclusion, we developed a strategy for comprehensive characterization of peptide ligase specificity that utilizes proteome-derived peptide libraries as ligase substrates. We used this strategy to characterize >25,000 enzyme-substrate pairs in the context of subtiligase and identified subtiligase variants with activity toward N-terminal sequences that were previously recalcitrant to modification. We applied these mutants individually for site-specific bioconjugation and in combinations for sequencing of the cellular N-terminome. These studies provide both a new toolbox of enzymes for site-specific protein modification and a general approach for rapidly defining and engineering peptide ligase specificity.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

INFORMAL SEQUENCE LISTING

SEQ ID NO: 1
AQSVPYGVSQIKAPALHSQGYTGSNVKVAVIDSGIDSSHPDLKVAGGAS
MVPSETNPFQDNNSHGTHVAGTVAALNNSIGVLGVAPSASLYAVKVLGA
DGSGQYSWIINGIEWAIANNMDVINMSLGGPSGSAALKAAVDKAVASGV
VVVAAAGNEGTSGSSSTVGYPGKYPSVIAVGAVDSSNQRASFSSVGPEL
DVMAPGVSIQSTLPGNKYGAYNGTCMASAHVAGAAALILSKHPNWTNTQ
VRSSLENTTTKLGDSFYYGKGLINVQAAAQ
SEQ ID NO: 2
AQSVPYGVSQIKAPALHSQGYTGSNVKVAVIDSGIDSSHPDLKVAGGAS
FVPSETNPFQDNNSHGTHVAGTVAALDNSIGVLGVAPSASLYAVKVLGA
DGSGQYSWIISGIEWAIANNMDVINMSLGGPSGSAALKAAVDKAVASGV
VVVAAAGNEGTSGSSSTVGYPGKYPSVIAVGAVDSSNQRASFSSVGPEL
DVMAPGVSIQSTLPGNRYGAYSGTCMASAHVAGAAALILSKHPNWTNTQ
VRSSLENTTTKLGDSFYYGKGLINVQAAAQ
SEQ ID NO: 3
AQSVPYGVSQIKAPALHSQGYTGSNVKVAVIDSGIDSSHPDLKVAGGAS
FVPSETNPFQDNNSHGTHVAGTVAALDNSIGVLGVAPSASLYAVKVLGA
DGSGQYSWIISGIEWAIANNMDVINLALGGPSGSAALKAAVDKAVASGV
VVVAAAGNEGTSGSSSTVGYPGKYPSVIAVGAVDSSNQRASFSSVGPEL
DVMAPGVSIQSTLPGNRYGAYSGTCMASAHVAGAAALILSKHPNWTNTQ
VRSSLENTTTKLGDSFYYGKGLINVQAAAQ
SEQ ID NO: 4
AQSVPYGVSQIKAPALHSQGYTGSNVKVAVIDSGIDSSHPDLNVAGGAS
FVPSETNPFQDNNSHGTHVAGTVLAVAPSASLYAVKVLGADGSGQYSWI
INGIEWAIANNMDVINMSLGGPSGSAALKAAVDKAVASGVVVVAAAGNE
GTSGSSSTVGYPGKYPSVIAVGAVDSSNQRASFSSVGPELDVMAPGVSI
VSTLPGNKYGAKSGTCMASAHVAGAAALILSKHPNWTNTQVRSSLENTT
TKLGDSFYYGKGLINVEAAAQ
SEQ ID NO: 5
ENLYFQSY
SEQ ID NO: 6
ENLYFQSK
SEQ ID NO: 7
ENLYFQSA
SEQ ID NO: 8
AAPY
SEQ ID NO: 9
AAPK
SEQ ID NO: 10
AAPA
SEQ ID NO: 11
EXXYXQ(S/G/A),
where X corresponds to any amino acid
SEQ ID NO: 12
E(T/V)LFQGP
SEQ ID NO: 13
DDDDK
SEQ ID NO: 14
DDDDK
SEQ ID NO: 15
(D/E)GR
SEQ ID NO: 15
LVPR
SEQ ID NO: 16
RXXR,
where X corresponds to any amino acid
SEQ ID NO: 17
IEPD
SEQ ID NO: 18
EEENLYFQ
SEQ ID NO: 19
ENLYFQ
SEQ ID NO: 20
AAPF-glc-FG
SEQ ID NO: 21
GGGS
SEQ ID NO: 22
AAPC-glc-FG
SEQ ID NO: 23
KAAPF-glc-FG
SEQ ID NO: 24-SEQ ID NO: 59
nucleic acid sequences in FIG. 22A
SEQ ID NO: 60-SEQ ID NO: 83
nucleic acid sequences in FIG. 22B
SEQ ID NO: 84
GTGAGAGGCAAAAAAGTATGGATCAGTTTGCTGTTTGCTTTAGCGTTAA
TCTTTACGATGGCGTTCGGCAGCACATCCTCTGCCCAGGCGGCCGGTAA
ATCCAACGGTGAGAAAAAATATATTGTAGGCTTCAAACAAACCATGAGC
ACCATGTCGGCTGCCAAAAAAAAAGACGTCATTTCAGAGAAGGGTGGGA
AGGTACAAAAACAGTTCAAATATGTAGATGCGGCCTCCGCCACGTTGAA
CGAAAAGGCGGTAAAAGAACTGAAAAAAGATCCGTCAGTGGCATACGTA
GAAGAAGATCATGTCGCGCATGCTTATGCTCAAAGCGTCCCGTACGGCG
TCTCACAGATCAAGGCACCGGCGCTGCACAGCCAGGGTTATACCGGCTC
CAACGTTAAGGTGGCGGTCATTGATAGCGGCATCGATAGCTCCCATCCC
GACCTCAAAGTTGCCGGCGGCGCTTCTATGGTGCCAAGCGAAACTAATC
CTTTTCAGGATAATAATAGTCACGGGACGCATGTAGCAGGTACAGTCGC
CGCTTTGAATAATTCTATCGGCGTGCTGGGTGTTGCGCCGAGCGCGTCA
CTCTACGCCGTGAAAGTGCTGGGCGCGGACGGCAGCGGACAATATAGTT
GGATTATTAATGGCATCGAGTGGGCCATCGCGAACAATATGGATGTGAT
CAATATGAGCCTGGGCGGCCCAAGCGGCAGTGCTGCCTTAAAAGCGGCG
GTGGATAAAGCTGTGGCAAGTGGGGTCGTCGTGGTGGCAGCGGCGGGCA
ATGAAGGCACGAGTGGCTCTTCTTCGACTGTCGGATACCCCGGCAAATA
CCCGTCGGTCATCGCGGTTGGGGCGGTTGATAGCTCTAACCAACGTGCC
AGTTTTAGCAGTGTAGGCCCAGAATTAGATGTGATGGCGCCAGGTGTGT
CTATCCAGAGCACACTCCCGGGCAATAAATATGGTGCGTATAATGGCAC
ATGTATGGCCAGTGCGCACGTTGCCGGGGCGGCGGCCCTGATCTTAAGT
AAACATCCAAACTGGACCAACACCCAGGTGCGTAGCAGTTTGGAAAACA
CCACCACGAAACTGGGTGATTCTTTTTATTACGGGAAAGGTCTCATCAA
TGTTCAAGCGGCCGCCCAACTCGAGCACCACCACCACCACCACTAA
SEQ ID NO: 85
AQSVPYGVSQIKAPALHSQGYTGSNVKVAVIDSGIDSSHPDLKVAGGAS
FVPSETNPFQDNNSHGTHVAGTVAALDNSIGVLGVAPSASLYAVKVLGA
DGSGQYSWIISGIEWAIANNMDVINLALGGPSGSAALKAAVDKAVASGV
VVVAAAGNEGTSGSSSTVGYPGKYPSVIAVGAVDSSNQRASFSSVGPEL
DVMAPGVSIQSTLPGNRYGAYSGTCMASAHVAGAAALILSKHPNWTNTQ
VRSSLENTTTKLGDSFYYGKGLINVQAAAQLEHHHHHH
SEQ ID NO: 86
GAAPF-glc-RK
SEQ ID NO: 87
AFAK
SEQ ID NO: 88
GAAPFAFAK

What is claimed is:

1. A subtiligase variant with an altered N-terminal protein substrate specificity or an improved aminolysis-to-hydrolysis (A/H) ratio compared to a wild-type subtiligase or a wild-type stabiligase.

2. The subtiligase variant of claim 1, wherein the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, M222, and a combination thereof, numbered in accordance with wild-type subtiligase.

3. The subtiligase variant of claim 1, wherein the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, Y217, M222, and a combination thereof, numbered in accordance with wild-type subtiligase.

4. The subtiligase variant of claim 1, wherein the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/K/R/W, M222A, and a combination thereof, numbered in accordance ith wild-type subtiligase.

5. The subtiligase variant of claim 4, wherein the one or more amino acid substitutions is selected from the group consisting of F189A/K/Q/R/S and Y217A/D/E/K/R/W.

6. The subtiligase variant of claim 5, wherein the amino acid substitutions are F189A/K/Q/R/S and Y217A/D/E/K/R/W.

7. The subtiligase variant of claim 5 or 6, wherein the subtiligase variant has the amino acid substitution M222A.

8. The subtiligase variant of claim 1, wherein the subtiligase variant comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/R/W, M222A, and a combination thereof, numbered in accordance with wild-type subtiligase.

9. The subtiligase variant of claim 8, wherein the one or more amino acid substitutions are selected from the group consisting of F189A/K/Q/R/S and Y217A/D/E/R/W.

10. The subtiligase variant of claim 9, wherein the amino acid substitutions are F189A/K/Q/R/S and Y217A/D/E/R/W.

11. The subtiligase variant of claim 9 or 10 , wherein the subtiligase variant has the amino acid substitution M222A.

12. The subtiligase variant of any one of claims 1 to 11, wherein the subtiligase variant catalyzes ligation of the N-terminus of said protein substrate and a synthetic molecule comprising a peptide ester.

13. The subtiligase variant of any one of claims 1 to 11, wherein the subtiligase variant catalyzes ligation of the N-terminus of said protein substrate and a synthetic molecule comprising a peptide thioester.

14. The subtiligase variant of any one of claims 1 to 13, wherein the altered N-terminal protein substrate specificity comprises an increased specificity for an acidic amino acid residue at the P1′ and/or the P2′ position of said protein substrate.

15. The subtiligase variant of any one of claims 1 to 13, wherein the altered N-terminal protein substrate specificity comprises an increased specificity for a His, Lys, Ser or Arg residue at the P1′ position of said protein substrate.

16. The subtiligase variant of any one of claims 1 to 13, wherein the altered N-terminal protein substrate specificity comprises an increased specificity for an aromatic, hydrophobic, polar, or acidic amino acid residue at the P1′ position and an acidic, basic, polar, or proline amino acid residue at the P2′ position of said protein substrate.

17. A nucleic acid encoding the subtiligase variant of any one of claims 1 to 16.

18. An expression vector comprising the nucleic acid of claim 17.

19. A host cell comprising the expression vector of claim 18.

20. A kit comprising the subtiligase variant of any one of claims 1 to 16, the nucleic acid of claim 17, the expression vector of claim 18, or the host cell of claim 19.

21. The kit of claim 20, further comprising a synthetic molecule for conjugating to the N-terminus of a protein substrate.

22. The kit of claim 21, wherein the synthetic molecule is a peptide ester or a peptide thioester.

23. The kit of claim 22, wherein the peptide ester or peptide thioester comprises a detectable moiety, therapeutic moiety, chemical moiety, drug moiety, binding moiety, nucleic acid, or reactive group.

24. A method of conjugating a synthetic molecule to the N-terminus of a protein substrate comprising contacting a protein substrate having a free α-amino group with one or more subtiligase variants having an altered N-terminal protein specificity or an improved A/H ratio compared to a wild-type subtiligase or a wild-type stabiligase, and a synthetic molecule under conditions to form a peptide bond between the synthetic molecule and the N-terminus of the protein substrate.

25. The method of claim 24, wherein the one or more subtiligase variants comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, M222, and a combination thereof, numbered in accordance with wild-type subtiligase.

26. The method of claim 24, wherein the one or more subtiligase variants comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60, N62, S63, H67, S125, L126, F189, Y217, M222, and a combination thereof, numbered in accordance with wild-type subtiligase.

27. The method of claim 24, wherein the one or more subtiligase variants comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/K/R/W, M222A, and a combination thereof, numbered in accordance with wild-type subtiligase.

28. The method of claim 27, wherein the one or more amino acid substitutions is selected from the group consisting of F189A/K/Q/R/S and Y217A/D/E/K/R/W.

29. The method of claim 28, wherein the amino acid substitutions are F189A/K/Q/R/S and Y217A/D/E/K/R/W.

30. The subtiligase variant of claim 28 or 29, wherein the one or more subtiligase variants has the amino acid substitution M222A.

31. The method of claim 24, wherein the one or more subtiligase variants comprises an amino acid sequence that is at least 90% identical to the amino acid sequence of wild-type subtiligase, wild-type stabiligase, SEQ ID NO:1, SEQ ID NO:2, SEQ ID NO:3, SEQ ID NO:4, or a fragment thereof, and has one or more amino acid substitutions selected from the group consisting of D60A, N62A, S63A, H67A, S125A, L126A, F189A/K/Q/R/S, Y217A/D/E/R/W, M222A, and a combination thereof, numbered in accordance with wild-type subtiligase.

32. The method of claim 31, wherein the one or more amino acid substitutions are selected from the group consisting of F189A/K/Q/R/S and Y217A/D/E/R/W.

33. The method of claim 32, wherein the amino acid substitutions are F189A/K/Q/R/S and Y217A/D/E/R/W.

34. The method of claim 32 or 33 , wherein the one or more subtiligase variants has the amino acid substitution M222A.

35. The method of any one of claims 24 to 34, wherein the synthetic molecule is a peptide ester or a peptide thioester.

36. The method of claim 35, wherein the peptide ester or the peptide thioester comprises a detectable moiety, therapeutic moiety, chemical moiety, drug moiety, binding moiety, nucleic acid, or reactive group.

37. The method of claim 35 or 36, wherein the protein ester or the protein thioester further comprises an amino acid sequence comprising at least one unnatural amino acid residue.

38. The method of any one of claims 24 to 37, wherein the protein substrate is present in a complex mixture.

39. The method of any one of claims 24 to 38, wherein the complex mixture is a biological sample.

40. The method of claim 39, wherein the biological sample is a cell lysate, tissue extract, whole intact cells, whole blood, plasma, serum or other biological fluid.


SEQUENCE LISTING (88 sequences)

SEQ ID NO: 1 - protein, 275 aa, Artificial Sequence
  Synthetic peptide
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGAYNGTCMASAHVAGAAALILSKHPNWTNTQVRSSLENTTTKLGDSFYYGKGLINVQAAAQ

SEQ ID NO: 2 - protein, 275 aa, Artificial Sequence
  Synthetic peptide
AQSVPYGVSQIKAPALHSQGYTGSNVKVAVIDSGIDSSHPDLKVAGGASFVPSETNPFQDNNSHGTHVAGTVAALDNSIGVLGVAPSASLYAVKVLGADGSGQYSWIISGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNRYGAYSGTCMASAHVAGAAALILSKHPNWTNTQVRSSLENTTTKLGDSFYYGKGLINVQAAAQ

SEQ ID NO: 3 - protein, 275 aa, Artificial Sequence
  Synthetic peptide
AQSVPYGVSQIKAPALHSQGYTGSNVKVAVIDSGIDSSHPDLKVAGGASFVPSETNPFQDNNSHGTHVAGTVAALDNSIGVLGVAPSASLYAVKVLGADGSGQYSWIISGIEWAIANNMDVINLALGGPSGSAALKAAVDKAVASGVVVVAAAGNEGTSGSSSTVGYPGKYPSVIAVGAVDSSNQRASFSSVGPELDVMAPGVSIQSTLPGNRYGAYSGTCMASAHVAGAAALILSKHPNWTNTQVRSSLENTTTKLGDSFYYGKGLINVQAAAQ

SEQ ID NO: 4 - protein, 266 aa, Artificial Sequence
  Synthetic peptide
AQSVPYGVSQIKAPALHSQGYTGSNVKVAVIDSGIDSSHPDLNVAGGASFVPSETNPFQDNNSHGTHVAGTVLAVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIVSTLPGNKYGAKSGTCMASAHVAGAAALILSKHPNWTNTQVRSSLENTTTKLGDSFYYGKGLINVEAAAQ

SEQ ID NO: 5 - protein, 8 aa, Artificial Sequence
  Synthetic peptide
ENLYFQSY

SEQ ID NO: 6 - protein, 8 aa, Artificial Sequence
  Synthetic peptide
ENLYFQSK

SEQ ID NO: 7 - protein, 8 aa, Artificial Sequence
  Synthetic peptide
ENLYFQSA

SEQ ID NO: 8 - protein, 4 aa, Artificial Sequence
  Synthetic peptide
AAPY

SEQ ID NO: 9 - protein, 4 aa, Artificial Sequence
  Synthetic peptide
AAPK

SEQ ID NO: 10 - protein, 4 aa, Artificial Sequence
  Synthetic peptide
AAPA

SEQ ID NO: 11 - protein, 9 aa, Artificial Sequence
  Synthetic peptide
  misc_feature (2)..(3): Xaa can be any naturally occurring amino acid
  misc_feature (5)..(5): Xaa can be any naturally occurring amino acid
EXXYXQSGA

SEQ ID NO: 12 - protein, 8 aa, Artificial Sequence
  Synthetic peptide
ETVLFQGP

SEQ ID NO: 13 - protein, 5 aa, Artificial Sequence
  Synthetic peptide
DDDDK

SEQ ID NO: 14 - protein, 5 aa, Artificial Sequence
  Synthetic peptide
DDDDK

SEQ ID NO: 15 - protein, 4 aa, Artificial Sequence
  Synthetic peptide
DEGR

SEQ ID NO: 16 - protein, 4 aa, Artificial Sequence
  Synthetic peptide
  misc_feature (2)..(3): Xaa can be any naturally occurring amino acid
RXXR

SEQ ID NO: 17 - protein, 4 aa, Artificial Sequence
  Synthetic peptide
IEPD

SEQ ID NO: 18 - protein, 8 aa, Artificial Sequence
  Synthetic peptide
EEENLYFQ

SEQ ID NO: 19 - protein, 6 aa, Artificial Sequence
  Synthetic peptide
ENLYFQ

SEQ ID NO: 20 - protein, 9 aa, Artificial Sequence
  Synthetic peptide
AAPFGLCFG

SEQ ID NO: 21 - protein, 4 aa, Artificial Sequence
  Synthetic peptide
GGGS

SEQ ID NO: 22 - protein, 9 aa, Artificial Sequence
  Synthetic peptide
AAPCGLCFG

SEQ ID NO: 23 - protein, 10 aa, Artificial Sequence
  Synthetic peptide
KAAPFGLCFG

SEQ ID NO: 24 - DNA, 59 bp, Artificial Sequence
  Synthetic primer
aatgaaaaaaaggagaggataaagagtgagaggcaaaaaagtatggatcagtttgctgt

SEQ ID NO: 25 - DNA, 52 bp, Artificial Sequence
  Synthetic primer
cggggccaaggccggttttttatgtttagtggtggtggtggtggtgctcgag

SEQ ID NO: 26 - DNA, 25 bp, Artificial Sequence
  Synthetic primer
cctggtgccgcgcggcagccatatg

SEQ ID NO: 27 - DNA, 41 bp, Artificial Sequence
  Synthetic primer
ccttagaaaccattccaccaatctgttctctgtgagcctca

SEQ ID NO: 28 - DNA, 45 bp, Artificial Sequence
  Synthetic primer
cagattggtggaatggtttctaagggtgaagaattgttcaccgga

SEQ ID NO: 29 - DNA, 86 bp, Artificial Sequence
  Synthetic primer
ggtggtggtgctcgagtgcggccgcaagcttgtcgacggagctcgaattcggattacttatacagctcatccattcccagggtgat

SEQ ID NO: 30 - DNA, 51 bp, Artificial Sequence
  Synthetic primer
cagattggtggagcgtttatggtttctaagggtgaagaattgttcaccgga

SEQ ID NO: 31 - DNA, 40 bp, Artificial Sequence
  Synthetic primer
aaccataaacgctccaccaatctgttctctgtgagcctca

SEQ ID NO: 32 - DNA, 51 bp, Artificial Sequence
  Synthetic primer
cagattggtggagaatttatggtttctaagggtgaagaattgttcaccgga

SEQ ID NO: 33 - DNA, 41 bp, Artificial Sequence
  Synthetic primer
aaaccataaattctccaccaatctgttctctgtgagcctca

SEQ ID NO: 34 - DNA, 50 bp, Artificial Sequence
  Synthetic primer
cagattggtggagattttatggtttctaagggtgaagaattgttcaccgg

SEQ ID NO: 35 - DNA, 41 bp, Artificial Sequence
  Synthetic primer
aaaccataaaatctccaccaatctgttctctgtgagcctca

SEQ ID NO: 36 - DNA, 51 bp, Artificial Sequence
  Synthetic primer
cagattggtggagcggaaatggtttctaagggtgaagaattgttcaccgga

SEQ ID NO: 37 - DNA, 41 bp, Artificial Sequence
  Synthetic primer
aaaccatttccgctccaccaatctgttctctgtgagcctca

SEQ ID NO: 38 - DNA, 51 bp, Artificial Sequence
  Synthetic primer
cagattggtggagcggatatggtttctaagggtgaagaattgttcaccgga

SEQ ID NO: 39 - DNA, 41 bp, Artificial Sequence
  Synthetic primer
aaaccatatccgctccaccaatctgttctctgtgagcctca

SEQ ID NO: 40 - DNA, 33 bp, Artificial Sequence
  Synthetic primer
gatgccgctatcaatggccgccaccttaacgtt

SEQ ID NO: 41 - DNA, 33 bp, Artificial Sequence
  Synthetic primer
aacgttaaggtggcggccattgatagcggcatc

SEQ ID NO: 42 - DNA, 34 bp, Artificial Sequence
  Synthetic primer
gggatgggagctatcggcgccgctatcaatgacc

SEQ ID NO: 43 - DNA, 34 bp, Artificial Sequence
  Synthetic primer
ggtcattgatagcggcgccgatagctcccatccc

SEQ ID NO: 44 - DNA, 46 bp, Artificial Sequence
  Synthetic primer
tacatgcgtcccgtgactattagcatcctgaaaaggattagtttcg

SEQ ID NO: 45 - DNA, 46 bp, Artificial Sequence
  Synthetic primer
cgaaactaatccttttcaggatgctaatagtcacgggacgcatgta

SEQ ID NO: 46 - DNA, 29 bp, Artificial Sequence
  Synthetic primer
cgactgtacctgctgcatgcgtcccgtga

SEQ ID NO: 47 - DNA, 29 bp, Artificial Sequence
  Synthetic primer
tcacgggacgcatgcagcaggtacagtcg

SEQ ID NO: 48 - DNA, 39 bp, Artificial Sequence
  Synthetic primer
gccgcccaggctcgcattgatcacatccatattgttcgc

SEQ ID NO: 49 - DNA, 39 bp, Artificial Sequence
  Synthetic primer
gcgaacaatatggatgtgatcaatgcgagcctgggcggc

SEQ ID NO: 50 - DNA, 34 bp, Artificial Sequence
  Synthetic primer
atttgccgggggctccgacagtcgaagaagagcc

SEQ ID NO: 51 - DNA, 34 bp, Artificial Sequence
  Synthetic primer
ggctcttcttcgactgtcggagcccccggcaaat

SEQ ID NO: 52 - DNA, 25 bp, Artificial Sequence
  Synthetic primer
tcaaccgccccagccgcgatgaccg

SEQ ID NO: 53 - DNA, 25 bp, Artificial Sequence
  Synthetic primer
cggtcatcgcggctggggcggttga

SEQ ID NO: 54 - DNA, 40 bp, Artificial Sequence
  Synthetic primer
catacatgtgccagcatacgcaccatatttattgcccggg

SEQ ID NO: 55 - DNA, 40 bp, Artificial Sequence
  Synthetic primer
cccgggcaataaatatggtgcgtatgctggcacatgtatg

SEQ ID NO: 56 - DNA, 33 bp, Artificial Sequence
  Synthetic primer
gcactggccatacatgcgccattatacgcacca

SEQ ID NO: 57 - DNA, 33 bp, Artificial Sequence
  Synthetic primer
tggtgcgtataatggcgcatgtatggccagtgc

SEQ ID NO: 58 - DNA, 34 bp, Artificial Sequence
  Synthetic primer
gcgcactggccgcacatgtgccattatacgcacc

SEQ ID NO: 59 - DNA, 34 bp, Artificial Sequence
  Synthetic primer
ggtgcgtataatggcacatgtgcggccagtgcgc

SEQ ID NO: 60 - DNA, 30 bp, Artificial Sequence
  Synthetic primer
cggcaacgtgcgcagcggccatacatgtgc

SEQ ID NO: 61 - DNA, 30 bp, Artificial Sequence
  Synthetic primer
gcacatgtatggccgctgcgcacgttgccg

SEQ ID NO: 62 - DNA, 28 bp, Artificial Sequence
  Synthetic primer
cgccccggcaacggccgcactggccata

SEQ ID NO: 63 - DNA, 28 bp, Artificial Sequence
  Synthetic primer
tatggccagtgcggccgttgccggggcg

SEQ ID NO: 64 - DNA, 41 bp, Artificial Sequence
  Synthetic primer
gcgtcccgtgactattattagcctgaaaaggattagtttcg

SEQ ID NO: 65 - DNA, 41 bp, Artificial Sequence
  Synthetic primer
cgaaactaatccttttcaggctaataatagtcacgggacgc

SEQ ID NO: 66 - DNA, 42 bp, Artificial Sequence
  Synthetic primer
gcgtcccgtgactagcattatcctgaaaaggattagtttcgc

SEQ ID NO: 67 - DNA, 42 bp, Artificial Sequence
  Synthetic primer
gcgaaactaatccttttcaggataatgctagtcacgggacgc

SEQ ID NO: 68 - DNA, 46 bp, Artificial Sequence
  Synthetic primer
acatgcgtcccgtgagcattattatcctgaaaaggattagtttcgc

SEQ ID NO: 69 - DNA, 46 bp, Artificial Sequence
  Synthetic primer
gcgaaactaatccttttcaggataataatgctcacgggacgcatgt

SEQ ID NO: 70 - DNA, 40 bp, Artificial Sequence
  Synthetic primer
acaatatggatgtgatcaatatggccctgggcggcccaag

SEQ ID NO: 71 - DNA, 40 bp, Artificial Sequence
  Synthetic primer
cttgggccgcccagggccatattgatcacatccatattgt

SEQ ID NO: 72 - DNA, 38 bp, Artificial Sequence
  Synthetic primer
gcgactgtacctgctacagccgtcccgtgactattatt

SEQ ID NO: 73 - DNA, 38 bp, Artificial Sequence
  Synthetic primer
aataatagtcacgggacggctgtagcaggtacagtcgc

SEQ ID NO: 74 - DNA, 34 bp, Artificial Sequence
  Synthetic primer
cgcttgggccgcccgcgctcatattgatcacatc

SEQ ID NO: 75 - DNA, 34 bp, Artificial Sequence
  Synthetic primer
gatgtgatcaatatgagcgcgggcggcccaagcg

SEQ ID NO: 76 - DNA, 36 bp, Artificial Sequence
  Synthetic primer
ctgggcctacactgctagcactggcacgttggttag

SEQ ID NO: 77 - DNA, 36 bp, Artificial Sequence
  Synthetic primer
ctaaccaacgtgccagtgctagcagtgtaggcccag

SEQ ID NO: 78 - DNA, 40 bp, Artificial Sequence
  Synthetic primer
ggccatacatgtgccattagccgcaccatatttattgccc

SEQ ID NO: 79 - DNA, 40 bp, Artificial Sequence
  Synthetic primer
gggcaataaatatggtgcggctaatggcacatgtatggcc

SEQ ID NO: 80 - DNA, 45 bp, Artificial Sequence
  Synthetic primer
  misc_feature (23)..(24): n is a, c, g, or t
actggccatacatgtgccattmnncgcaccatatttattgcccgg

SEQ ID NO: 81 - DNA, 45 bp, Artificial Sequence
  Synthetic primer
  misc_feature (22)..(23): n is a, c, g, or t
ccgggcaataaatatggtgcgnnkaatggcacatgtatggccagt

SEQ ID NO: 82 - DNA, 44 bp, Artificial Sequence
  Synthetic primer
  misc_feature (16)..(17): n is a, c, g, or t
gggcctacactgctmnnactggcacgttggttagagctatcaac

SEQ ID NO: 83 - DNA, 44 bp, Artificial Sequence
  Synthetic primer
  misc_feature (28)..(29): n is a, c, g, or t
gttgatagctctaaccaacgtgccagtnnkagcagtgtaggccc

SEQ ID NO: 84 - DNA, 1173 bp, Artificial Sequence
  Synthetic primer
gtgagaggcaaaaaagtatggatcagtttgctgtttgctttagcgttaatctttacgatggcgttcggcagcacatcctctgcccaggcggccggtaaatccaacggtgagaaaaaatatattgtaggcttcaaacaaaccatgagcaccatgtcggctgccaaaaaaaaagacgtcatttcagagaagggtgggaaggtacaaaaacagttcaaatatgtagatgcggcctccgccacgttgaacgaaaaggcggtaaaagaactgaaaaaagatccgtcagtggcatacgtagaagaagatcatgtcgcgcatgcttatgctcaaagcgtcccgtacggcgtctcacagatcaaggcaccggcgctgcacagccagggttataccggctccaacgttaaggtggcggtcattgatagcggcatcgatagctcccatcccgacctcaaagttgccggcggcgcttctatggtgccaagcgaaactaatccttttcaggataataatagtcacgggacgcatgtagcaggtacagtcgccgctttgaataattctatcggcgtgctgggtgttgcgccgagcgcgtcactctacgccgtgaaagtgctgggcgcggacggcagcggacaatatagttggattattaatggcatcgagtgggccatcgcgaacaatatggatgtgatcaatatgagcctgggcggcccaagcggcagtgctgccttaaaagcggcggtggataaagctgtggcaagtggggtcgtcgtggtggcagcggcgggcaatgaaggcacgagtggctcttcttcgactgtcggataccccggcaaatacccgtcggtcatcgcggttggggcggttgatagctctaaccaacgtgccagttttagcagtgtaggcccagaattagatgtgatggcgccaggtgtgtctatccagagcacactcccgggcaataaatatggtgcgtataatggcacatgtatggccagtgcgcacgttgccggggcggcggccctgatcttaagtaaacatccaaactggaccaacacccaggtgcgtagcagtttggaaaacaccaccacgaaactgggtgattctttttattacgggaaaggtctcatcaatgttcaagcggccgcccaactcgagcaccaccaccaccaccactaa

SEQ ID NO: 85 - protein, 283 aa, Artificial Sequence
  Synthetic peptide
AQSVPYGVSQIKAPALHSQGYTGSNVKVAVIDSGIDSSHPDLKVAGGASFVPSETNPFQDNNSHGTHVAGTVAALDNSIGVLGVAPSASLYAVKVLGADGSGQYSWIISGIEWAIANNMDVINLALGGPSGSAALKAAVDKAVASGVVVVAAAGNEGTSGSSSTVGYPGKYPSVIAVGAVDSSNQRASFSSVGPELDVMAPGVSIQSTLPGNRYGAYSGTCMASAHVAGAAALILSKHPNWTNTQVRSSLENTTTKLGDSFYYGKGLINVQAAAQLEHHHHHH

SEQ ID NO: 86 - protein, 10 aa, Artificial Sequence
  Synthetic peptide
GAAPFGLCRK

SEQ ID NO: 87 - protein, 4 aa, Artificial Sequence
  Synthetic peptide
AFAK

SEQ ID NO: 88 - protein, 9 aa, Artificial Sequence
  Synthetic peptide
GAAPFAFAK